(19) United States
(12) Patent Application Publication
(10) Pub. No.: US 2021/0332354 A1
(43) Pub. Date: Oct. 28, 2021

(54) SYSTEMS AND METHODS FOR IDENTIFYING DIFFERENTIAL ACCESSIBILITY OF GENE REGULATORY ELEMENTS AT SINGLE CELL RESOLUTION

(71) Applicant: 10x Genomics, Inc., Pleasanton, CA (US)

(72) Inventors: Preyas Shah, San Jose, CA (US); Li Wang, Pleasanton, CA (US); Brett Olsen, Alameda, CA (US)

(21) Appl. No.: 17/231,972

(22) Filed: Apr. 15, 2021

Related U.S. Application Data
(60) Provisional application No. 63/010,561, filed on Apr. 15, 2020.

(51) Int. Cl. C12N 15/10; C12Q 1/6806; C12Q 1/6876; G16B 30/10

(52) U.S. Cl. CPC C12N 15/1093; G16B 30/10; C12Q 1/6876; C12Q 1/6806

(57) ABSTRACT

A method for ascertaining differential accessibility of (TF) binding motifs in open chromatin regions of cells, comprising receiving a cell barcode genomic sequence dataset; aligning each of a plurality of fragment sequence reads to a reference sequence; identifying peaks defined by the aligned plurality of fragment sequence reads; generating a peak-barcode matrix that is comprised of peaks for each cell barcode; clustering cells with peaks having similar chromatin accessibility profiles into a cell cluster to form one or more cell clusters; generating a TF barcode matrix that maps each peak in the peak-barcode matrix to one or more given TF binding motif(s); performing a differential accessibility analysis, wherein the analysis identifies differences in accessibility of peaks and TF binding motifs associated with each identified cell cluster relative to all other identified cell clusters; and generating an output of one or more refined cell clusters based on the differential accessibility analysis.

### CROSS REFERENCE

This application claims priority benefit of the filing date of U.S. Provisional Patent Application No. 63/010,561, filed on Apr. 15, 2020, the disclosure of which application is herein incorporated by reference in its entirety.

### FIELD

This description is generally directed towards systems and methods for identifying differential accessibility of gene regulatory elements in transposase accessible open chromatin regions using multi-modal droplet-based single cell genomic sequencing technologies. More specifically, systems and methods for identifying the differential accessibility of gene regulatory elements at a single cell level for understanding the regulatory landscape and networks of the genome and cellular epigenetic variability, are disclosed herein.

### BACKGROUND

In eukaryotic genomes, chromosomal DNA winds itself around histone proteins (i.e., “nucleosomes”), thereby forming a complex known as chromatin. The tight or loose packaging of chromatin contributes to the control of gene expression. Tightly packed chromatin (“closed chromatin”) is usually not permissive for gene expression, while more loosely packaged, accessible regions of chromatin (“open chromatin”) is associated with the active transcription of gene products. Methods for probing genome-wide DNA accessibility have proven extremely effective in identifying regulatory elements across a variety of cell types and quantifying changes that lead to both activation or repression of gene expression.

One such method is the Assay for Transposase Accessible Chromatin with high-throughput sequencing (ATAC-seq). The ATAC-seq method probes DNA accessibility with an artificial transposon, which inserts specific sequences into accessible regions of chromatin. Because the transposase can only insert sequences into accessible regions of chromatin not bound by transcription factors and/or nucleosomes, sequencing reads can be used to infer regions of increased chromatin accessibility.

Traditional approaches to the ATAC-seq methodology requires large pools of cells, processes cells in bulk, and result in data representative of an entire cell population, but lack information about cell-to-cell variation inherently present in a cell population (see, e.g., Buenrostro, et al., Curr. Protoc. Mol. Biol., 2015 Jan. 5; 21.29.1-21.29.9). While single cell ATAC-seq (scATAC-seq) methods have been developed, they suffer from several limitations. For example, scATAC-seq methods that utilize sample pooling, cell indexing, and cell sorting (see, e.g., Cusanovich, et al., Science, 2015 May 22; 348(6237):910-14) result in high variability and a low number of reads associated with any single cell. Other scATAC-seq methods that utilize a programmable microfluidic device to isolate single cells and perform scATAC-seq in nanoliter reaction chambers (see, e.g., Buenrostro, et al., Nature, 2015 Jul. 23; 523(7561):486-90) are limited by the throughput of the assay and may not generate personal epigenomic profiles on a timescale compatible with clinical decision-making.

As such, there is a need for systems and methods that can utilize multi-modal droplet-based single cell genomic sequencing technologies for identifying, analyzing, and visualizing the differential accessibility of gene regulatory landscape and networks across the genome at a high resolution. Information gained from such systems and methods can provide insights into how differential chromatin compaction and binding of DNA-binding proteins (e.g., transcription factors) regulate gene expression, including gene regulatory networks that are upstream of gene expression, defining cell types and states, and for understanding cellular heterogeneity stemming from epigenetic variability.

### SUMMARY

In accordance with various embodiments, a method for ascertaining differential accessibility of transcription factor (TF) binding motifs in open chromatin regions of cells using a cell barcode genomic sequence dataset, is provided. The method can include receiving, by one or more processors, the cell barcode genomic sequence dataset, wherein the dataset comprises a plurality of fragment sequence reads and associated barcodes. The method can further include aligning, by the one or more processors, each of the plurality of fragment sequence reads to a reference sequence. The method can further include identifying, by the one or more processors, one or more peaks defined by the aligned plurality of fragment sequence reads, each peak representing an enrichment of the aligned fragment sequence reads at a given position on the reference sequence, wherein peaks that produce a signal above a pre-set signal threshold demarcates an open chromatin region. The method can further include generating, by the one or more processors, a peak-barcode matrix that is comprised of peaks for each cell barcode. The method can further include clustering, by the one or more processors, cells with peaks having similar chromatin accessibility profiles based on one or more given parameters into a cell cluster to form one or more cell clusters. The method can further include generating, by the one or more processors, a transcription factor (TF) barcode matrix that maps each peak in the peak-barcode matrix to one or more given TF binding motif(s). The method can further include performing, by the one or more processors, a differential accessibility analysis, wherein the analysis identifies differences in accessibility of one or more peaks and TF binding motifs associated with each identified cell cluster relative to all other identified cell clusters. The method can further include generating, by the one or more processors, an output of one or more refined cell clusters based on the differential accessibility analysis, wherein the output comprises a differential accessibility of gene regulatory function and/or specific TF binding motifs associated with each refined cell cluster.

In accordance with various embodiments, a non-transitory computer-readable medium in which a program is stored for causing a computer to perform a method for ascertaining differential accessibility of transcription factor (TF) binding motifs in open chromatin regions of cells using a cell barcode genomic sequence dataset, is provided. The method can include receiving, by one or more processors, the cell barcode genomic sequence dataset, wherein the dataset comprises a plurality of fragment sequence reads and associated barcodes. The method can further include aligning, by the one or more processors, each of the plurality of fragment sequence reads to a reference sequence. The method can further include identifying, by the one or more processors, one or more peaks defined by the aligned plurality of fragment sequence reads, each peak representing an enrichment of the aligned fragment sequence reads at a given position on the reference sequence, wherein peaks that produce a signal above a pre-set signal threshold demarcates an open chromatin region. The method can further include generating, by the one or more processors, a peak-barcode matrix that is comprised of peaks for each cell barcode. The method can further include clustering, by the one or more processors, cells with peaks having similar chromatin accessibility profiles based on one or more given parameters into a cell cluster to form one or more cell clusters. The method can further include generating, by the one or more processors, a transcription factor (TF) barcode matrix that maps each peak in the peak-barcode matrix to one or more given TF binding motif(s). The method can further include performing, by the one or more processors, a differential accessibility analysis, wherein the analysis identifies differences in accessibility of one or more peaks and TF binding motifs associated with each identified cell cluster relative to all other identified cell clusters. The method can further include generating, by the one or more processors, an output of one or more refined cell clusters based on the differential accessibility analysis, wherein the output comprises a differential accessibility of gene regulatory function and/or specific TF binding motifs associated with each refined cell cluster.

In accordance with various embodiments, a system for ascertaining differential accessibility of transcription factor binding motifs in open chromatin regions of cells using a cell barcode genomic sequence dataset, is provided. The system includes a data store, a computing device and a display. The data store is configured to store the cell barcode genomic sequence dataset comprising a plurality of fragment sequence reads and associated barcodes. The computing device is communicatively connected to the data store and hosts a clustering engine, a TF barcode matrix engine, and a differential analysis engine. The clustering engine is configured to: receive the cell barcode genomic sequence dataset, align each of the plurality of fragment sequence reads to a reference sequence, identify one or more peaks defined by the aligned plurality of fragment sequence reads, each peak representing an enrichment of the aligned fragment sequence reads at a given position on the reference sequence, wherein peaks that produce a signal above a pre-set signal threshold demarcates an open chromatin region, generate a peak-barcode matrix that is comprised of peaks for each cell barcode, and cluster cells with peaks having similar chromatin accessibility profiles based on one or more given parameters into a cell cluster to form one or more cell clusters. In various embodiments, the clustering parameter is the closeness of the distance metric calculated using the cut sites in the peaks of the cells. The TF barcode matrix engine is configured to generate a transcription factor barcode matrix (TF-barcode matrix) that maps each peak in the peak-barcode matrix to one or more given TF binding motif(s). The differential analysis engine is configured to: perform a differential accessibility analysis to identify differences in accessibility of one or more peaks and TF binding motifs associated with each identified cell cluster relative to all other identified cell clusters, and generate an output of one or more refined cell clusters based on the differential accessibility analysis, wherein the output comprises a differential accessibility of gene regulatory function and/or specific TF binding motifs associated with each refined cell cluster. The display is communicatively connected to the computing device and configured to display a report containing the output of one or more refined cell clusters.

These and other aspects and implementations are discussed in detail herein. The foregoing information and the following detailed description include illustrative examples of various aspects and implementations, and provide an overview or framework for understanding the nature and character of the claimed aspects and implementations. The drawings provide illustration and a further understanding of the various aspects and implementations, and are incorporated in and constitute a part of this specification.

### BRIEF DESCRIPTION OF FIGURES

The accompanying drawings are not intended to be drawn to scale. Like reference numbers and designations in the various drawings indicate like elements. For purposes of clarity, not every component may be labeled in every drawing. In the drawings:

FIG. 1 is a high level illustration of the gene regulatory elements in transposase accessible open chromatin regions.

FIG. 2 is a schematic illustration of a non-limiting example of the sequencing workflow for using single cell Assay for Transposase Accessible Chromatin (ATAC) sequencing to generate sequencing data for identifying genome-wide differential accessibility of gene regulatory elements, in accordance with various embodiments.

FIG. 3 is a schematic illustration of a non-limiting example of the sequencing data analysis workflow for analyzing single cell ATAC sequencing data for identifying genome-wide differential accessibility of gene regulatory elements, in accordance with various embodiments.

FIG. 4 is an illustration of exemplary genome tracks and peaks from analysis of single cell ATAC sequencing profiles, in accordance with various embodiments.

FIG. 5 is a plot that illustrates how transposition events are measured with a 401 bp moving window sum around each base-pair along the genome, in accordance with various embodiments.

FIG. 6 is a barcode rank plot that demonstrates the distribution of barcode counts and which barcodes were inferred to be associated with cells, in accordance with various embodiments.

FIG. 7 is an illustration of exemplary heatmap of Z-scores of TF motifs in single cell ATAC sequencing cell clusters, in accordance with various embodiments.

FIG. 8 is a flowchart illustrating a non-limiting example method for ascertaining differential accessibility of transcription factor (TF) binding motifs in open chromatin regions of cells using a cell barcode genomic sequence dataset, in accordance with various embodiments, in accordance with various embodiments.

FIG. 9 is a diagram illustrating a non-limiting example system for ascertaining differential accessibility of transcription factor (TF) binding motifs in open chromatin regions of cells using a cell barcode genomic sequence dataset, in accordance with various embodiments.

FIG. 10 is a block diagram that illustrates a computer system, upon which embodiments, or portions of the embodiments, may be implemented, in accordance with various embodiments.

FIG. 11 is a collection of plots demonstrating that GC bias exists due to excess GC-rich peaks in some cell types causing GC-rich transcription factor (TF) motifs to be excessively, strongly differential.

FIG. 12 are plots demonstrating that GC content and peak size both influence chromatin accessibility analysis, in accordance with various embodiments.

FIG. 13 are plots demonstrating non-limiting examples of testing for outlier peaks via Fisher's exact test, in accordance with various embodiments.

FIGS. 14A and 14B are charts are illustrating the improvement in peak calling versus publicly available methods.

FIG. 15 provides multiple charts illustrating receiver-operator characteristic (ROC) curves comparing current and new methods for peak calling, in accordance with various embodiments.

It is to be understood that the figures are not necessarily drawn to scale, nor are the objects in the figures necessarily drawn to scale in relationship to one another. The figures are depictions that are intended to bring clarity and understanding to various embodiments of apparatuses, systems, and methods disclosed herein. Wherever possible, the same reference numbers will be used throughout the drawings to refer to the same or like parts. Moreover, it should be appreciated that the drawings are not intended to limit the scope of the present teachings in any way.

### DETAILED DESCRIPTION

The following description of various embodiments is exemplary and explanatory only and is not to be construed as limiting or restrictive in any way. Other embodiments, features, objects, and advantages of the present teachings will be apparent from the description and accompanying drawings, and from the claims.

This specification describes various exemplary embodiments of methods for conducting a customized analysis of open chromatin regions on a cell using a barcode genomic sequence dataset. It should be appreciated, however, that although the systems and methods disclosed herein refer to their application in open chromatin analysis specifically, they are equally applicable to other analogous fields.

In various embodiments, a computer program product can include instructions to receive an output file for a cell barcode genomic sequence dataset, the output file having various components such as a peak-barcode matrix and one or more cell clusters; instructions to adjust one of more customizable parameters for analyzing the output file (e.g., peak barcode matrix), and instructions to generate an updated output file including an updated clustering of cells, based on the one or more customizable parameters, wherein each updated cell cluster includes cells with peaks representing a specific gene regulatory function.

In various embodiments, a system for conducting a customized analysis of open chromatin regions on a cell using a barcode genomic sequence dataset is provided and can include a data source for receiving, by one or more processors, an output file for the cell barcode genomic sequence dataset and one or more computing devices that can host and execute software code that comprises a clustering engine (and optionally a TF-barcode matrix engine and differential analysis engine). The clustering engine can be configured to generate an updated output file including updated clustering of cells, based on the one or more customizable parameters, wherein each updated cell cluster includes cells with peaks representing a specific gene regulatory function.

The disclosure, however, is not limited to these exemplary embodiments and applications or to the manner in which the exemplary embodiments and applications operate or are described herein. Moreover, the figures may show simplified or partial views, and the dimensions of elements in the figures may be exaggerated or otherwise not in proportion. In addition, as the terms “on,” “attached to,” “connected to,” “coupled to,” or similar words are used herein, one element (e.g., a material, a layer, a substrate, etc.) can be “on,” “attached to,” “connected to,” or “coupled to” another element regardless of whether the one element is directly on, attached to, connected to, or coupled to the other element or there are one or more intervening elements between the one element and the other element. In addition, where reference is made to a list of elements (e.g., elements a, b, c), such reference is intended to include any one of the listed elements by itself, any combination of less than all of the listed elements, and/or a combination of all of the listed elements. Section divisions in the specification are for ease of review only and do not limit any combination of elements discussed.

It should be understood that any use of subheadings herein are for organizational purposes, and should not be read to limit the application of those subheaded features to the various embodiments herein. Each and every feature described herein is applicable and usable in all the various embodiments discussed herein and that all features described herein can be used in any contemplated combination, regardless of the specific example embodiments that are described herein. It should further be noted that exemplary description of specific features are used, largely for informational purposes, and not in any way to limit the design, subfeature, and functionality of the specifically described feature.

All publications mentioned herein are incorporated herein by reference for the purpose of describing and disclosing devices, compositions, formulations and methodologies which are described in the publication and which might be used in connection with the present disclosure.

As used herein, “substantially” means sufficient to work for the intended purpose. The term “substantially” thus allows for minor, insignificant variations from an absolute or perfect state, dimension, measurement, result, or the like such as would be expected by a person of ordinary skill in the field but that do not appreciably affect overall performance. When used with respect to numerical values or parameters or characteristics that can be expressed as numerical values, “substantially” means within ten percent.

The term “ones” means more than one.

As used herein, the term “plurality” can be 2, 3, 4, 5, 6, 7, 8, 9, 10, or more.

As used herein, the terms “comprise”, “comprises”, “comprising”, “contain”, “contains”, “containing”, “have”, “having” “include”, “includes”, and “including” and their variants are not intended to be limiting, are inclusive or open-ended and do not exclude additional, un-recited additives, components, integers, elements or method steps. For example, a process, method, system, composition, kit, or apparatus that comprises a list of features is not necessarily limited only to those features but may include other features not expressly listed or inherent to such process, method, system, composition, kit, or apparatus.

Where values are described as ranges, it will be understood that such disclosure includes the disclosure of all possible sub-ranges within such ranges, as well as specific numerical values that fall within such ranges irrespective of whether a specific numerical value or specific sub-range is expressly stated.

Unless otherwise defined, scientific and technical terms used in connection with the present teachings described herein shall have the meanings that are commonly understood by those of ordinary skill in the art. Further, unless otherwise required by context, singular terms shall include pluralities and plural terms shall include the singular. Generally, nomenclatures utilized in connection with, and techniques of, chemistry, biochemistry, pharmacology and toxicology, cell and tissue culture, molecular biology, and protein and oligo- or polynucleotide chemistry and hybridization described herein are those well known and commonly used in the art. Standard techniques are used, for example, for nucleic acid purification and preparation, chemical analysis, recombinant nucleic acid, and oligonucleotide synthesis. Enzymatic reactions and purification techniques are performed according to manufacturer's specifications or as commonly accomplished in the art or as described herein. The techniques and procedures described herein are generally performed according to conventional methods well known in the art and as described in various general and more specific references that are cited and discussed throughout the instant specification. See, e.g., Sambrook et al., Molecular Cloning: A Laboratory Manual (Third ed., Cold Spring Harbor Laboratory Press, Cold Spring Harbor, N.Y. 2000). The nomenclatures utilized in connection with, and the laboratory procedures and techniques described herein are those well known and commonly used in the art.

DNA (deoxyribonucleic acid) is a chain of nucleotides consisting of 4 types of nucleotides; A (adenine), T (thymine), C (cytosine), and G (guanine), and that RNA (ribonucleic acid) is comprised of 4 types of nucleotides; A, U (uracil), G, and C. Certain pairs of nucleotides specifically bind to one another in a complementary fashion (called complementary base pairing). That is, adenine (A) pairs with thymine (T) (in the case of RNA, however, adenine (A) pairs with uracil (U)), and cytosine (C) pairs with guanine (G). When a first nucleic acid strand binds to a second nucleic acid strand made up of nucleotides that are complementary to those in the first strand, the two strands bind to form a double strand. As used herein, “nucleic acid sequencing data,” “nucleic acid sequencing information,” “nucleic acid sequence,” “genomic sequence,” “genetic sequence,” or “fragment sequence,” or “nucleic acid sequencing read” denotes any information or data that is indicative of the order of the nucleotide bases (e.g., adenine, guanine, cytosine, and thymine/uracil) in a molecule (e.g., whole genome, whole transcriptome, exome, oligonucleotide, polynucleotide, fragment, etc.) of DNA or RNA. It should be understood that the present teachings contemplate sequence information obtained using all available varieties of techniques, platforms or technologies, including, but not limited to: capillary electrophoresis, microarrays, ligation-based systems, polymerase-based systems, hybridization-based systems, direct or indirect nucleotide identification systems, pyrosequencing, ion- or pH-based detection systems, electronic signature-based systems, etc.

A “polynucleotide”, “nucleic acid”, or “oligonucleotide” refers to a linear polymer of nucleosides (including deoxyribonucleosides, ribonucleosides, or analogs thereof) joined by internucleosidic linkages. Typically, a polynucleotide comprises at least three nucleosides. Usually oligonucleotides range in size from a few monomeric units, e.g. 3-4, to several hundreds of monomeric units. Whenever a polynucleotide such as an oligonucleotide is represented by a sequence of letters, such as “ATGCCTG,” it will be understood that the nucleotides are in 5′->3′ order from left to right and that “A” denotes deoxyadenosine, “C” denotes deoxycytidine, “G” denotes deoxyguanosine, and “T” denotes thymidine, unless otherwise noted. The letters A, C, G, and T may be used to refer to the bases themselves, to nucleosides, or to nucleotides comprising the bases, as is standard in the art.

The phrase “next generation sequencing” (NGS) refers to sequencing technologies having increased throughput as compared to traditional Sanger- and capillary electrophoresis-based approaches, for example with the ability to generate hundreds of thousands of relatively small sequence reads at a time. Some examples of next generation sequencing techniques include, but are not limited to, sequencing by synthesis, sequencing by ligation, and sequencing by hybridization. More specifically, the MISEQ, HISEQ, NEXTSEQ, NOVASEQ Systems of Illumina, the GRIDION and PROMETHION Systems of Oxford Nanopore Technologies, PACBIO SEQUEL Systems of Pacific Biosciences, and the Personal Genome Machine (PGM) and SOLiD Sequencing System of Life Technologies Corp, provide massively parallel sequencing of whole or targeted genomes. The SOLiD System and associated workflows, protocols, chemistries, etc. are described in more detail in PCT Publication No. WO 2006/084132, entitled “Reagents, Methods, and Libraries for Bead-Based Sequencing,” international filing date Feb. 1, 2006, U.S. patent application Ser. No. 12/873,190, entitled “Low-Volume Sequencing System and Method of Use,” filed on Aug. 31, 2010, and U.S. patent application Ser. No. 12/873,132, entitled “Fast-Indexing Filter Wheel and Method of Use,” filed on Aug. 31, 2010, the entirety of each of these applications being incorporated herein by reference thereto.

The phrase “sequencing run” refers to any step or portion of a sequencing experiment performed to determine some information relating to at least one biomolecule (e.g., nucleic acid molecule).

The term “genome,” as used herein, generally refers to genomic information from a subject, which may be, for example, at least a portion or an entirety of a subject's hereditary information. A genome can be encoded either in DNA or in RNA. A genome can comprise coding regions (e.g., that code for proteins) as well as non-coding regions. A genome can include the sequence of all chromosomes together in an organism. For example, the human genome ordinarily has a total of 46 chromosomes. The sequence of all of these together may constitute a human genome.

As used herein, the phrase “genomic features” can refer to a genome region with some annotated function (e.g., a gene, protein coding sequence, mRNA, tRNA, rRNA, repeat sequence, inverted repeat, miRNA, siRNA, etc.) or a genetic/genomic variant (e.g., single nucleotide polymorphism/variant, insertion/deletion sequence, copy number variation, inversion, etc.) which denotes a single or a grouping of genes (in DNA or RNA) that have undergone changes as referenced against a particular species or sub-populations within a particular species due to mutations, recombination/crossover or genetic drift.

The term “sequencing,” as used herein, generally refers to methods and technologies for determining the sequence of nucleotide bases in one or more polynucleotides. The polynucleotides can be, for example, nucleic acid molecules such as deoxyribonucleic acid (DNA) or ribonucleic acid (RNA), including variants or derivatives thereof (e.g., single stranded DNA). Sequencing can be performed by various systems currently available, such as, without limitation, a sequencing system by Illumina®, Pacific Biosciences (PacBio®), Oxford Nanopore®, or Life Technologies (Ion Torrent®). Alternatively or in addition, sequencing may be performed using nucleic acid amplification, polymerase chain reaction (PCR)(e.g., digital PCR, quantitative PCR, or real time PCR), or isothermal amplification. Such systems may provide a plurality of raw genetic data corresponding to the genetic information of a subject (e.g., human), as generated by the systems from a sample provided by the subject.

In some examples, such systems provide “sequencing reads” (also referred to as “fragment sequence reads” or “reads” herein). A read may include a string of nucleic acid bases corresponding to a sequence of a nucleic acid molecule that has been sequenced. In some situations, systems and methods provided herein may be used with proteomic information.

In general, the methods and systems described herein accomplish targeted genomic sequencing by providing for the determination of the sequence of long individual nucleic acid molecules and/or the identification of direct molecular linkage as between two sequence segments separated by long stretches of sequence, which permit the identification and use of long range sequence information, but this sequencing information is obtained using methods that have the advantages of the extremely low sequencing error rates and high throughput of short read sequencing technologies. The methods and systems described herein segment long nucleic acid molecules into smaller fragments that can be sequenced using high-throughput, higher accuracy short-read sequencing technologies, and that segmentation is accomplished in a manner that allows the sequence information derived from the smaller fragments to retain the original long range molecular sequence context, i.e., allowing the attribution of shorter sequence reads to originating longer individual nucleic acid molecules. By attributing sequence reads to an originating longer nucleic acid molecule, one can gain significant characterization information for that longer nucleic acid sequence that one cannot generally obtain from short sequence reads alone. This long range molecular context is not only preserved through a sequencing process, but is also preserved through the targeted enrichment process used in targeted sequencing approaches described herein, where no other sequencing approach has shown this ability.

In general, sequence information from smaller fragments will retain the original long range molecular sequence context through the use of a tagging procedure, including the addition of barcodes as described herein and known in the art. In specific examples, fragments originating from the same original longer individual nucleic acid molecule will be tagged with a common barcode, such that any later sequence reads from those fragments can be attributed to that originating longer individual nucleic acid molecule. Such barcodes can be added using any method known in the art, including addition of barcode sequences during amplification methods that amplify segments of the individual nucleic acid molecules as well as insertion of barcodes into the original individual nucleic acid molecules using transposons, including methods such as those described in Amini et al., Nature Genetics 46: 1343-1349 (2014) (advance online publication on Oct. 29, 2014), which is hereby incorporated by reference in its entirety for all purposes and in particular for all teachings related to adding adaptor and other oligonucleotides using transposons. Once nucleic acids have been tagged using such methods, the resultant tagged fragments can be enriched using methods described herein such that the population of fragments represents targeted regions of the genome. As such, sequence reads from that population allows for targeted sequencing of select regions of the genome, and those sequence reads can also be attributed to the originating nucleic acid molecules, thus preserving the original long range molecular sequence context. The sequence reads can be obtained using any sequencing methods and platforms known in the art and described herein.

In addition to providing the ability to obtain sequence information from targeted regions of the genome, the methods and systems described herein can also provide other characterizations of genomic material, including without limitation haplotype phasing, identification of structural variations, and identifying copy number variations, as described in co-pending applications U.S. Ser. Nos. 14/752,589 and 14/752,602, (both filed on Jun. 26, 2015), which are herein incorporated by reference in their entirety for all purposes and in particular for all written description, figures and working examples directed to characterization of genomic material.

Methods of processing and sequencing nucleic acids in accordance with the methods and systems described in the present application are also described in further detail in U.S. Ser. Nos. 14/316,383; 14/316,398; 14/316,416; 14/316,431; 14/316,447; and 14/316,463 which are herein incorporated by reference in their entirety for all purposes and in particular for all written description, figures and working examples directed to processing nucleic acids and sequencing and other characterizations of genomic material.

The term “barcode,” as used herein, generally refers to a label, or identifier, that conveys or is capable of conveying information about an analyte. A barcode can be part of an analyte. A barcode can be independent of an analyte. A barcode can be a tag attached to an analyte (e.g., nucleic acid molecule) or a combination of the tag in addition to an endogenous characteristic of the analyte (e.g., size of the analyte or end sequence(s)). A barcode may be unique. Barcodes can have a variety of different formats. For example, barcodes can include barcode sequences, such as: polynucleotide barcodes; random nucleic acid and/or amino acid sequences; and synthetic nucleic acid and/or amino acid sequences. A barcode can be attached to an analyte in a reversible or irreversible manner. A barcode can be added to, for example, a fragment of a deoxyribonucleic acid (DNA) or ribonucleic acid (RNA) sample before, during, and/or after sequencing of the sample. Barcodes can allow for identification and/or quantification of individual sequencing reads.

As used herein, the term “cell barcode” refers to any barcodes that have been determined to be associated with a cell, as determined by a “cell calling” step within various embodiments of the disclosure.

As used herein, the term “Gel bead-in-EMulsion” or “GEM” refers to a droplet containing some sample volume and a barcoded gel bead, forming an isolated reaction volume. When referring to the subset of the sample contained in the droplet, the term “partition” may also be used. In various embodiments within the disclosure, the term barcode can refer to a GEM containing a gel bead that carries many DNA oligonucleotides with the same barcode, whereas different GEMs have different barcodes.

As used herein, the term “EM well” or “GEM group” refers to a set of partitioned cells (i.e., Gel beads-in-Emulsion or GEMs) from a single 10× Chromium™ Chip channel. One or more sequencing libraries can be derived from a GEM well.

The terms “adaptor(s)”, “adapter(s)” and “tag(s)” may be used synonymously. An adaptor or tag can be coupled to a polynucleotide sequence to be “tagged” by any approach, including ligation, hybridization, or other approaches. In various embodiments within the disclosure, the term adapter can refer to customized strands of nucleic acid base pairs created to bind with specific nucleic acid sequences, e.g., sequences of DNA.

The term “bead,” as used herein, generally refers to a particle. The bead may be a solid or semi-solid particle. The bead may be a gel bead. The gel bead may include a polymer matrix (e.g., matrix formed by polymerization or cross-linking). The polymer matrix may include one or more polymers (e.g., polymers having different functional groups or repeat units). Polymers in the polymer matrix may be randomly arranged, such as in random copolymers, and/or have ordered structures, such as in block copolymers. Cross-linking can be via covalent, ionic, or inductive, interactions, or physical entanglement. The bead may be a macromolecule. The bead may be formed of nucleic acid molecules bound together. The bead may be formed via covalent or non-covalent assembly of molecules (e.g., macromolecules), such as monomers or polymers. Such polymers or monomers may be natural or synthetic. Such polymers or monomers may be or include, for example, nucleic acid molecules (e.g., DNA or RNA). The bead may be formed of a polymeric material. The bead may be magnetic or non-magnetic. The bead may be rigid. The bead may be flexible and/or compressible. The bead may be disruptable or dissolvable. The bead may be a solid particle (e.g., a metal-based particle including but not limited to iron oxide, gold or silver) covered with a coating comprising one or more polymers. Such coating may be disruptable or dissolvable.

The term “macromolecule” or “macromolecular constituent,” as used herein, generally refers to a macromolecule contained within or from a biological particle. The macromolecular constituent may comprise a nucleic acid. In some cases, the biological particle may be a macromolecule. The macromolecular constituent may comprise DNA. The macromolecular constituent may comprise RNA. The RNA may be coding or non-coding. The RNA may be messenger RNA (mRNA), ribosomal RNA (rRNA) or transfer RNA (tRNA), for example. The RNA may be a transcript. The RNA may be small RNA that are less than 200 nucleic acid bases in length, or large RNA that are greater than 200 nucleic acid bases in length. Small RNAs may include 5.8S ribosomal RNA (rRNA), 5S rRNA, transfer RNA (tRNA), microRNA (miRNA), small interfering RNA (siRNA), small nucleolar RNA (snoRNAs), Piwi-interacting RNA (piRNA), tRNA-derived small RNA (tsRNA) and small rDNA-derived RNA (srRNA). The RNA may be double-stranded RNA or single-stranded RNA. The RNA may be circular RNA. The macromolecular constituent may comprise a protein. The macromolecular constituent may comprise a peptide. The macromolecular constituent may comprise a polypeptide.

The term “molecular tag,” as used herein, generally refers to a molecule capable of binding to a macromolecular constituent. The molecular tag may bind to the macromolecular constituent with high affinity. The molecular tag may bind to the macromolecular constituent with high specificity. The molecular tag may comprise a nucleotide sequence. The molecular tag may comprise a nucleic acid sequence. The nucleic acid sequence may be at least a portion or an entirety of the molecular tag. The molecular tag may be a nucleic acid molecule or may be part of a nucleic acid molecule. The molecular tag may be an oligonucleotide or a polypeptide. The molecular tag may comprise a DNA aptamer. The molecular tag may be or comprise a primer. The molecular tag may be, or comprise, a protein. The molecular tag may comprise a polypeptide. The molecular tag may be a barcode.

The term “partition,” as used herein, generally, refers to a space or volume that may be suitable to contain one or more species or conduct one or more reactions. A partition may be a physical compartment, such as a droplet or well. The partition may isolate space or volume from another space or volume. The droplet may be a first phase (e.g., aqueous phase) in a second phase (e.g., oil) immiscible with the first phase. The droplet may be a first phase in a second phase that does not phase separate from the first phase, such as, for example, a capsule or liposome in an aqueous phase. A partition may comprise one or more other (inner) partitions. In some cases, a partition may be a virtual compartment that can be defined and identified by an index (e.g., indexed libraries) across multiple and/or remote physical compartments. For example, a physical compartment may comprise a plurality of virtual compartments.

The term “subject,” as used herein, generally refers to an animal, such as a mammal (e.g., human) or avian (e.g., bird), or other organism, such as a plant. For example, the subject can be a vertebrate, a mammal, a rodent (e.g., a mouse), a primate, a simian or a human. Animals may include, but are not limited to, farm animals, sport animals, and pets. A subject can be a healthy or asymptomatic individual, an individual that has or is suspected of having a disease (e.g., cancer) or a pre-disposition to the disease, and/or an individual that is in need of therapy or suspected of needing therapy. A subject can be a patient. A subject can be a microorganism or microbe (e.g., bacteria, fungi, archaea, viruses).

The term “sample,” as used herein, generally refers to a “biological sample” of a subject. The sample may be obtained from a tissue of a subject. The sample may be a cell sample. A cell may be a live cell. The sample may be a cell line or cell culture sample. The sample can include one or more cells. The sample can include one or more microbes. The biological sample may be a nucleic acid sample or protein sample. The biological sample may also be a carbohydrate sample or a lipid sample. The biological sample may be derived from another sample. The sample may be a tissue sample, such as a biopsy, core biopsy, needle aspirate, or fine needle aspirate. The sample may be a fluid sample, such as a blood sample, urine sample, or saliva sample. The sample may be a skin sample. The sample may be a cheek swab. The sample may be a plasma or serum sample. The sample may be a cell-free or cell free sample. A cell-free sample may include extracellular polynucleotides. Extracellular polynucleotides may be isolated from a bodily sample that may be selected from the group consisting of blood, plasma, serum, urine, saliva, mucosal excretions, sputum, stool and tears. In some embodiments, the term “sample” can refer to a cell or nuclei suspension extracted from a single biological source (blood, tissue, etc.).

The sample may comprise any number of macromolecules, for example, cellular macromolecules. The sample may be or may include one or more constituents of a cell, but may not include other constituents of the cell. An example of such cellular constituents is a nucleus or an organelle. The sample may be or may include DNA, RNA, organelles, proteins, or any combination thereof. The sample may be or include a chromosome or other portion of a genome. The sample may be or may include a bead (e.g., a gel bead) comprising a cell or one or more constituents from a cell, such as DNA, RNA, nucleus, organelles, proteins, or any combination thereof, from the cell. The sample may be or may include a matrix (e.g., a gel or polymer matrix) comprising a cell or one or more constituents from a cell, such as DNA, RNA, nucleus, organelles, proteins, or any combination thereof, from the cell.

As used herein, the term “fragment” refers to unique ATAC-seq fragment captured by the ATAC-seq assay. Each fragment can be created by two separate transposition events, which create the two ends of the observed fragment. Each unique fragment may generate multiple duplicate reads. These duplicate reads can be collapsed into a single fragment record.

In some embodiments, the term “fragment” can also refer to a piece of genomic DNA, bound by two adjacent cut sites, that has been converted into a sequencer-compatible molecule with an attached cell-barcode. The alignment interval of such fragment can be obtained by correcting the alignment interval of the sequenced fragment by +4 base-pair (bp) on the left end of the fragment, and −5 bp on the right end (where left and right are relative to genomic coordinates). It is understood that, such correction is performed to account for the 9 bp of DNA that the transposase occupies when it cuts the DNA (accessibility of chromatin is recorded around the center of this 9 bp stretch). Most fragment-based metrics computed by the analysis workflow can be based on fragments that passed various quality filters.

As used herein, the term “nucleosome” refers to structural units of the DNA formed by histones that help package the eukaryotic DNA into well organized chromosomes.

As used herein, the term “histone” refers to protein found in eukaryotic cell nuclei that forms nucleosomes.

As used herein, the term “PCR duplicates” refers to duplicates created during PCR amplification. During PCR amplification of the fragments, each unique fragment that is created may result in multiple read-pairs sequenced with near identical barcodes and sequence data. These duplicate reads are identified computationally, and collapsed into a single fragment record for downstream analysis.

As used herein, the term “peak” refers to a compact region of the genome identified as having “open chromatin” due to an enrichment of cut-sites inside the region.

As used herein, the term “chromatin” refers to macromolecular complex formed by DNA, nucleosomes, and other proteins that bind DNA (for example transcription factors).

As used herein, the term “promoter” refers to a region of DNA that initiates transcription of a particular gene. Promoters can be located near the transcription start sites of genes, on the same strand and upstream on the DNA.

As used herein, the term “read data” refers to raw genomic data from sequenced DNA.

As used herein, the term “read-pair” refers to the read data sequenced from one molecule. This can include read1, read2, and the barcode sequence read.

As used herein, the term “sequencing run” refers to a flowcell containing data from one sequencing instrument run. The sequencing data can be further addressed by lane and by one or more sample indices.

As used herein, the “targeted region” refers to any known, annotated, epigenetically relevant regions in the genome such as transcription start sites (TSS), enhancers, promoters or DNase hypersensitive sites. The embodiment metrics often refer to these as targeted regions.

As used herein, the term “transcription Factor (TF)” refers to a protein that controls the rate of transcription of genetic information from DNA to messenger RNA, by binding to a specific DNA sequences (like promoter or enhancers) that are commonly located in the vicinity of the gene they control.

As used herein, the term “transposase enzyme” refers to an enzyme that can cut open chromatin and ligate adapters to the 3′ end of each DNA strand.

As used herein, the term “cut site” or “cut-site” refers to location on the genome where transposase cuts the DNA and inserts adapters.

As used herein, the term “transposition” refers to the reaction carried out by the transposase enzyme.

As used herein, the term “transcription start site” or “TSS” is the location where transcription starts at the 5′-end of a gene sequence.

Therefore, in accordance with various embodiments, various methods are provided that use single-cell genomic sequencing data to identify genome-wide differential accessibility of gene regulatory elements for providing insights into regulatory landscape and networks of the genome and cellular epigenetic variability.

### Open Chromatin Region Analysis Background

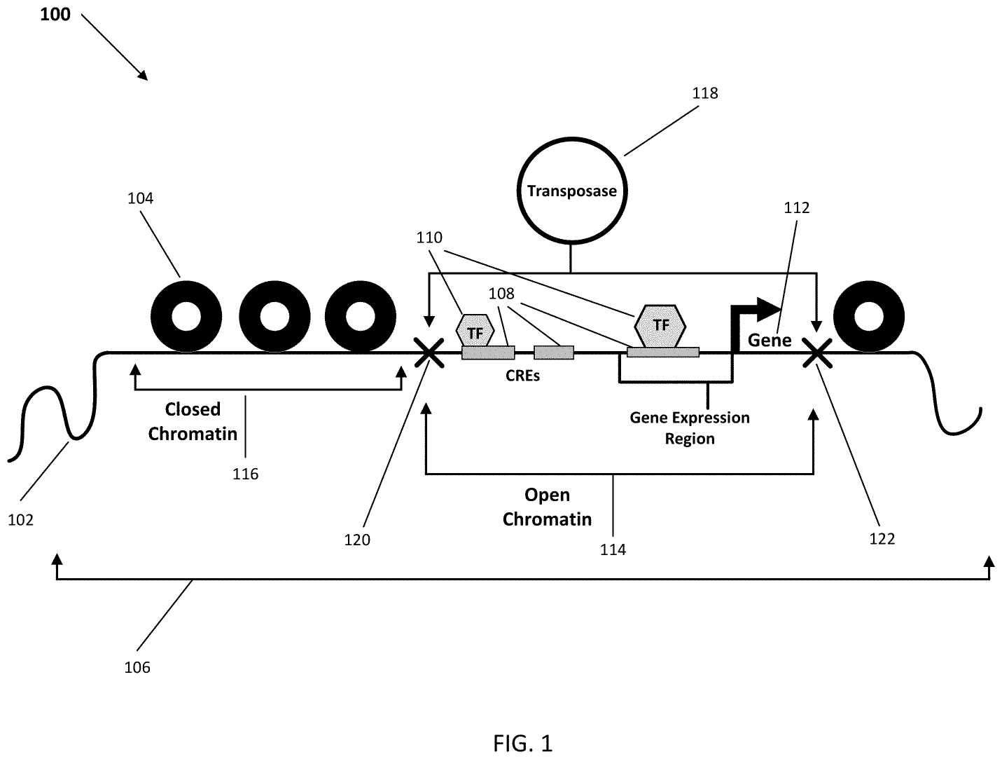

FIG. 1 is a high level illustration 100 of the gene regulatory elements in transposase accessible open chromatin regions. In eukaryotic cells, chromatin is the basic hereditary unit, which consists of DNA, histone proteins, and other genetic materials, and regulates cell type-specific gene expression. As shown herein, chromosomal DNA 102 winds itself around histone proteins, i.e., nucleosomes 104, thereby forming a complex known as chromatin 106. Briefly, regulation of transcription, i.e., gene expression, is a dynamic interaction between the chromatin and recruitment of numerous proteins (e.g., transcription factors (TFs), such as activators and repressors) to various gene regulatory elements on the DNA (e.g., enhancers, silencers, upstream activator sequences, promoters etc.). The TFs recruit the transcriptional machinery, which consists of various proteins including the core transcription protein, the RNA polymerase, to the gene expression region for the RNA polymerase to initiate transcription of a gene.

Generally, the gene regulatory elements can be classified into two types. As shown herein in FIG. 1, cis-regulatory element(s) (CREs) 108 are regions of non-coding DNA that can regulate the transcription of neighboring gene(s) 112. Whereas, trans-regulatory elements (TREs) are genes that can modify (or regulate) the expression of distant genes. Specifically, trans-regulatory elements are DNA sequences that can encode gene regulatory proteins, such as transcription factor(s) (TFs) 110, as shown in FIG. 1, which can then bind to the CREs 108 on the DNA and regulate the expression of the gene(s) 112, on the same or on another chromosome.

The tight or loose packaging of chromatin contributes to the regulation of gene expression. For example, loosely packaged, accessible regions of chromatin, also known as open chromatin and shown herein as open chromatin 114, can be associated with the active gene regulatory elements, i.e., the CREs 108 and TFs 110. This is because, in general, the regulatory elements selectively localize in the open chromatin 114, which is crucial to gene regulation. On the other hand, condensed chromatin, also known as closed chromatin and shown herein as closed chromatin 116, is generally not permissive for gene expression and restricts binding of the TFs 110 to the CREs 108.

Methods for probing genome-wide chromatin accessibility have proven extremely effective in identifying regulatory elements across a variety of cell types and quantifying changes that lead to both activation or repression of gene expression. One such method is the Assay for Transposase Accessible Chromatin with high-throughput sequencing (ATAC-seq) that utilizes a phenomenon known as DNA transposition to reveal accessible chromatin regions at a genome-wide level. DNA transposition is a phenomenon that transfers DNA sequence from one region of chromosome to another, which is assisted by DNA transposase. The ATAC-seq method probes chromatin accessibility with an artificial transposase 118, which can insert specific adapter sequences into accessible genomic DNA regions of the chromatin (shown herein as accessible DNA region 120 and 122) and tag the accessible DNA with such adapter sequences. Because the transposase can only insert sequences into accessible regions of the chromatin not bound by transcription factors and/or nucleosomes, sequencing the transposase tagged, accessible DNA fragments can be used to identify regions of increased chromatin accessibility and gene regulation.

With this understanding of the dynamic interaction between transposase accessible open chromatin regions and gene regulatory elements and the crucial role it can play in identifying gene expression regulation, it is valuable to be able to accurately identify, analyze, and visualize accessibility of gene regulatory elements for enabling deeper understanding of regulatory networks and cellular epigenetic variability for specific cells and multiple biological processes at a higher single cell resolution.

### Single Cell Assay for Transposase Accessible Chromatin (ATAC) Sequencing and Data Analysis Workflows

### Single Cell ATAC Sequencing Workflow

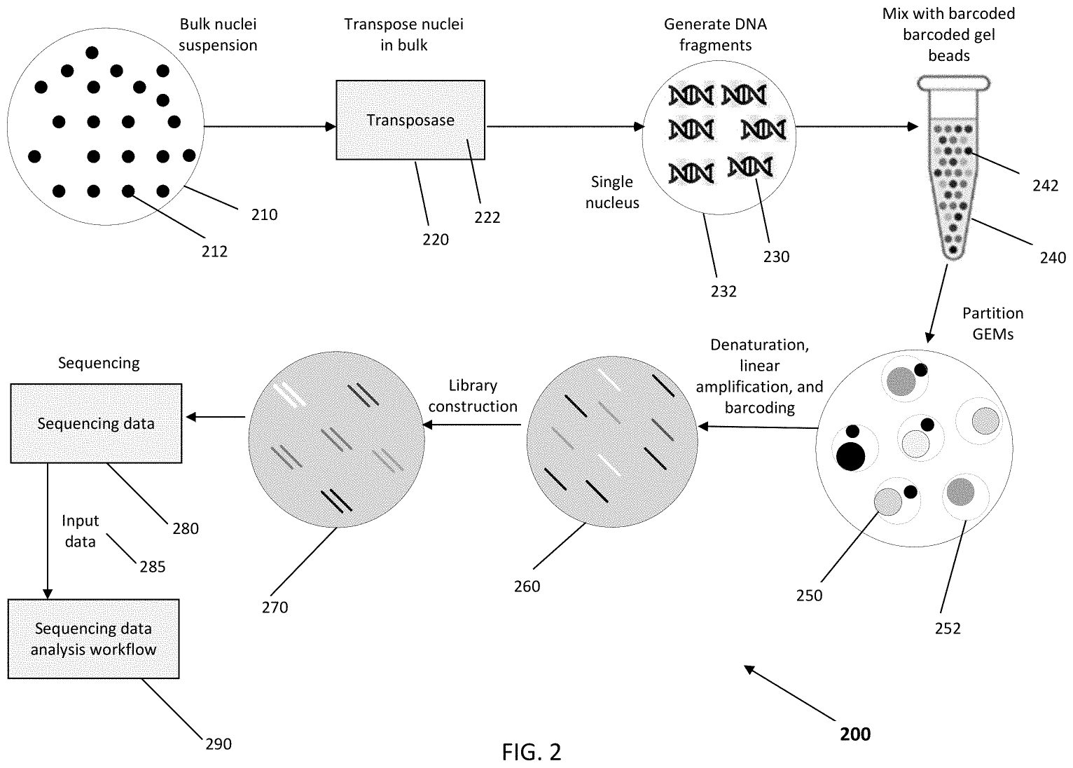

In accordance with various embodiments, a general schematic workflow is provided in FIG. 2 to illustrate a non-limiting example process for using single cell Assay for Transposase Accessible Chromatin (ATAC) sequencing technology to generate sequencing data. Such sequencing data can be used for identifying genome-wide differential accessibility of gene regulatory elements in accordance with various embodiments. The workflow can include various combinations of features, whether it be more or less features than that illustrated in FIG. 2. As such, FIG. 2 simply illustrates one example of a possible workflow.

Nuclei Isolation

FIG. 2 provides a schematic workflow 200, the workflow including a bulk nuclei suspension 210 from a sample comprising a plurality of individual nuclei 212. In various embodiments, obtaining a bulk nuclei suspension can include isolating nuclei in bulk from a sample. It is understood that one problem with generating ATAC sequencing datasets, is that the dataset may contain a large percentage of read sequences (also referred to as reads) from mitochondrial DNA. Various methods, in accordance with various embodiments herein, can be employed for ensuring low mitochondrial reads from samples and high quality nuclei sequencing data. Accordingly, in some embodiments, preparation of the bulk nuclei suspension can include carefully extracting nuclei from cells, while ensuring the mitochondria stays intact. Various known protocols can be employed to isolate, wash, count nuclei, and generate nuclei suspensions for use with the single cell ATAC sequencing protocol of the embodiments herein. Nuclei for generating the nuclei suspensions can be isolated from any cells. Such cells may include, but are not limited to, cells from fresh and cryopreserved cell lines, e.g., human and mouse cell lines, as well as more fragile primary cells. In various embodiments, such cells may include, any eukaryotic cells, i.e., a eukaryotic cell with a chromatin structure. In various embodiments, such cells may include, but are not limited to, immune cells (e.g., B cells and T cells), peripheral blood mononuclear cells (PBMCs), Bone Marrow Mononuclear Cells (BMMCs), skin cells, cancer cells, embryonic neurons, and adult neurons. In various embodiments, nuclei for generating the nuclei suspensions can be isolated from different human and mouse tissues. In various embodiments, nuclei for generating the nuclei suspensions can be isolated from different human and mouse tumor samples.

Transpose Nuclei in Bulk and Generate DNA Fragments

The workflow 200 provided in FIG. 2 further includes transposing the bulk nuclei suspension and generating adapter-tagged DNA fragments. The bulk nuclei suspension 210 is incubated with a transposition mix 220 containing Transposase 222. Upon incubation, the Transposase 222 enters individual nuclei 212 and preferentially fragments the DNA in open regions of a chromatin to generate a plurality of adapter-tagged DNA fragments 230 inside individual transposed nucleus 232.

In various embodiments, transposing the bulk nuclei suspensions can include incubating the nuclei suspension with a transposition mix that includes a Transposase enzyme, e.g., a Tn5 transposase. The transposase can be a mutated, hyperactive Tn5 transposase. In some embodiments, the transposase can be a Mu transposase. The transposase enters the nuclei and preferentially fragments the DNA in open regions of the chromatin by a process called transposing. More specifically, in various embodiments herein, the process results in transposing the nuclei in a bulk solution. Simultaneously during this process, adapter sequences can be added to the ends of the DNA fragments by the transposase, a process called tagmentation. This process results in adapter-tagged DNA fragments inside individually transposed nucleus. Accordingly, the transposase enzyme (e.g., a mutated, hyperactive Tn5 transposase) tagments the free, open regions of the DNA, producing many small fragments with adapters on either end. Such fragments can then be sequenced, as described in detail below, if two transposase enzymes cut at close (for example, <1 kb) locations in the same orientation, so the fragment between them has the correct set of adapters. If three transposase enzymes cut at nearby locations in the same orientation, two fragments are produced that share a cut site between them. Each of the fragments are then barcoded independently and sequenced in accordance with various embodiments described in detail below.

GEM Generation

The workflow 200 provided in FIG. 2 further includes Gel beads-in-EMulsion (GEMs) generation. With the adapter-tagged DNA fragments 230 in hand, the bulk nuclei suspension containing the individual transposed nucleus 232 is mixed with a gel beads solution 140 containing a plurality of individually barcoded gel beads 242. In various embodiments, this step results in partitioning the nuclei into a plurality of individual GEMs 250, each including a single transposed nucleus 232 that contains a plurality of adapter-tagged DNA fragments 230, and a barcoded gel bead 242. This step also results in a plurality of GEMS 252, each containing a barcoded gel bead 242 but no nuclei. Detail related to GEM generation, in accordance with various embodiments disclosed herein, is provided below.

In various embodiments, GEMs can be generated by combining barcoded gel beads, transposed nuclei containing the transposase adapter-tagged DNA fragments, and other reagents or a combination of biochemical reagents that may be necessary for the GEM generation process. Such reagents may include, but are not limited to, a combination of biochemical reagents (e.g., a master mix) suitable for GEM generation and partitioning oil. The barcoded gel beads 242 of the various embodiments herein may include a gel bead attached to oligonucleotides containing (i) an Illumina® P5 sequence (adapter sequence), (ii) a 16 nucleotide (nt) 10× Barcode, and (iii) a Read 1 (Read 1N) sequencing primer sequence. It is understood that other adapter, barcode, and sequencing primer sequences can be contemplated within the various embodiments herein.

In various embodiments, GEMS are generated by partitioning the transposed nuclei (containing the transposase adapter-tagged DNA fragments) using a microfluidic chip. The microfluidic chip of the various embodiments herein can be a Chromium Chip E. To achieve single nuclei resolution per GEM, the nuclei can be delivered at a limiting dilution, such that the majority (e.g., ˜90-99%) of the generated GEMs do not contains any nuclei, while the remainder of the generated GEMs largely contain a single nucleus.

Barcoding DNA Fragments

The workflow 200 provided in FIG. 2 further includes barcoding the adapter-tagged DNA fragments 230 for producing a plurality of uniquely barcoded single-stranded DNA fragments 260. Upon generation of the GEMs 250, the gel beads 242 can be dissolved releasing the various oligonucleotides of the embodiments described above, which are then mixed with the adapter-tagged DNA fragments 230 resulting in a plurality of uniquely barcoded single-stranded DNA fragments 260 following amplification of the GEMs 250. Detail related to generation of the plurality of uniquely barcoded single-stranded DNA fragments 260, in accordance with various embodiments disclosed herein, is provided below.

In various embodiments, upon generation of the GEMs 250, the gel beads 242 can be dissolved, and oligonucleotides of the various embodiments disclosed herein, containing the Illumina® P5 sequence (adapter sequence), an unique 10× Barcode, and Read 1 sequencing primer sequence can be released and mixed with the adapter-tagged DNA fragment and other reagents or a combination of biochemical reagents (e.g., a master mix necessary for the amplification process). Methods such as denaturation and linear amplification during thermal cycling of the GEMs or splinted ligation can then be performed to produce a plurality of uniquely barcoded single-stranded DNA fragments 260. In various embodiments herein, the plurality of uniquely barcoded single-stranded DNA fragments 260 can be 10× barcoded single-stranded DNA fragments. In one non-limiting example of the various embodiments herein, a pool of ˜750,000, 10× barcodes are utilized to uniquely index and barcode the transposed DNA fragments of each individual nucleus.

Accordingly, barcoded products of the various embodiments herein can include a plurality of 10× barcoded single-stranded DNA fragments generated during the thermal cycling process. In one non-limiting example of the various embodiments herein, each such 10× barcoded single-stranded DNA fragment can include a Illumina® P5 sequence (adapter sequence), a unique 10× barcode, a Read 1 sequencing primer sequence, a transposase adapter-tagged DNA fragment or insert, and a Read 2 (Read 2N)) sequencing primer sequence.

In various embodiments, after the amplification and barcoding process, the GEMs 250 are broken and pooled DNA fractions are recovered. The adapter-flanked, 10× barcoded DNA fragments are released from the droplets, i.e., the GEMs 250, and processed in bulk to complete library preparation for sequencing (e.g., next generation high throughput sequencing such as the single cell ATAC sequencing), as described in detail below. In various embodiments, following the amplification process, leftover biochemical reagents can be removed from the post-GEM reaction mixture. In one embodiment of the disclosure, silane magnetic beads can be used to remove leftover biochemical reagents. Additionally, in accordance with embodiments herein, the unused barcodes from the sample can be eliminated, for example, by Solid Phase Reversible Immobilization (SPRI) beads.

Library Construction

The workflow 200 provided in FIG. 2 further includes a library construction step. In the library construction step of workflow 200, a library 270 containing a plurality of double-stranded DNA fragments are generated. These double-stranded DNA fragments can be utilized for completing the subsequent sequencing step, e.g., the single cell ATAC sequencing step. Detail related to the library construction, in accordance with various embodiments disclosed herein, is provided below.

In accordance with various embodiments disclosed herein, an Illumina® P7 sequence (adapter sequence) and a sample index (SI) sequence (e.g., i7) can be added during the library construction step via PCR to generate the library 270, which contains a plurality of double stranded DNA fragments. In accordance with various embodiments herein, the sample index sequences can each comprise of one or more oligonucleotides. In one embodiment, the sample index sequences can each comprise of four oligonucleotides. In various embodiments, when analyzing the single cell ATAC sequencing data for a given sample, the reads associated with all four of the oligonucleotides in the sample index can be combined for identification of a sample. Accordingly, in one non-limiting example, the final single cell ATAC sequencing libraries contain sequencer compatible double-stranded DNA fragments containing the P5 and P7 sequences used in Illumina® bridge amplification, a unique 10× barcode sequence, and Read 1 and Read 2 sequencing primer sequences.

Various embodiments of single cell ATAC sequencing technology within the disclosure can at least include platforms such as One Sample, One GEM Well, One Flowcell; One Sample, One GEM well, Multiple Flowcells; One Sample, Multiple GEM Wells, One Flowcell; Multiple Samples, Multiple GEM Wells, One Flowcell; and Multiple Samples, Multiple GEM Wells, Multiple Flowcells platform. Accordingly, various embodiments within the disclosure can include sequence dataset from one or more samples, samples from one or more donors, and multiple libraries from one or more donors.

Sequencing

The workflow 200 provided in FIG. 2 further includes a sequencing step. In this step, the library 270 can be sequenced to generate a plurality of sequencing data 280. The fully constructed library 270 can be sequenced according to a suitable sequencing technology, such as a next-generation sequencing protocol, to generate the sequencing data 280. In various embodiments, the next-generation sequencing protocol utilizes the Illumina® sequencer for generating the sequencing data. It is understood that other next-generation sequencing protocols, platforms, and sequencers such as, e.g., MiSeq™, NextSeq™ 500/550 (High Output), HiSeq 2500™ (Rapid Run), HiSeq™ 3000/4000, and NovaSeq™, can be also used with various embodiments herein.

Sequencing Data Input and Data Analysis Workflow

The workflow 200 provided in FIG. 2 further includes a sequencing data analysis workflow 290. With the sequencing data 280 in hand, the data can then be output, as desired, and used as an input data 285 for the downstream sequencing data analysis workflow 290 for identifying differential accessibility of gene regulatory elements in transposase accessible open chromatin regions, in accordance with various embodiments herein. Sequencing the single cell ATAC libraries produces standard output sequences (also referred to as the “single cell ATAC sequencing data”, “sequencing data”, “sequence data”, or the “sequence output data”) that can then be used as the input data 285, in accordance with various embodiments herein. The sequence data contains sequenced fragments (also interchangeably referred to as “fragment sequence reads”, “sequencing reads” or “reads”), which in various embodiments include DNA sequences of the transposase adapter-tagged fragments containing the associated 10× barcode sequences, adapter sequences, and primer oligo sequences.

The various embodiments, systems and methods within the disclosure further include processing and inputting the sequence data. A compatible format of the sequencing data of the various embodiments herein can be a FASTQ file. Other file formats for inputting the sequence data is also contemplated within the disclosure herein. Various software tools within the embodiments herein can be employed for processing and inputting the sequencing output data into input files for the downstream data analysis workflow. One example of a software tool that can process and input the sequencing data for downstream data analysis workflow is the cellranger-atac mkfastq tool within the Cell Ranger™ ATAC analysis pipeline. It is understood that, various systems and methods with the embodiments herein are contemplated that can be employed to independently analyze the inputted single cell ATAC sequencing data for identifying genome-wide differential accessibility of gene regulatory elements in accordance with various embodiments.

### Single Cell ATAC Data Analysis Workflow

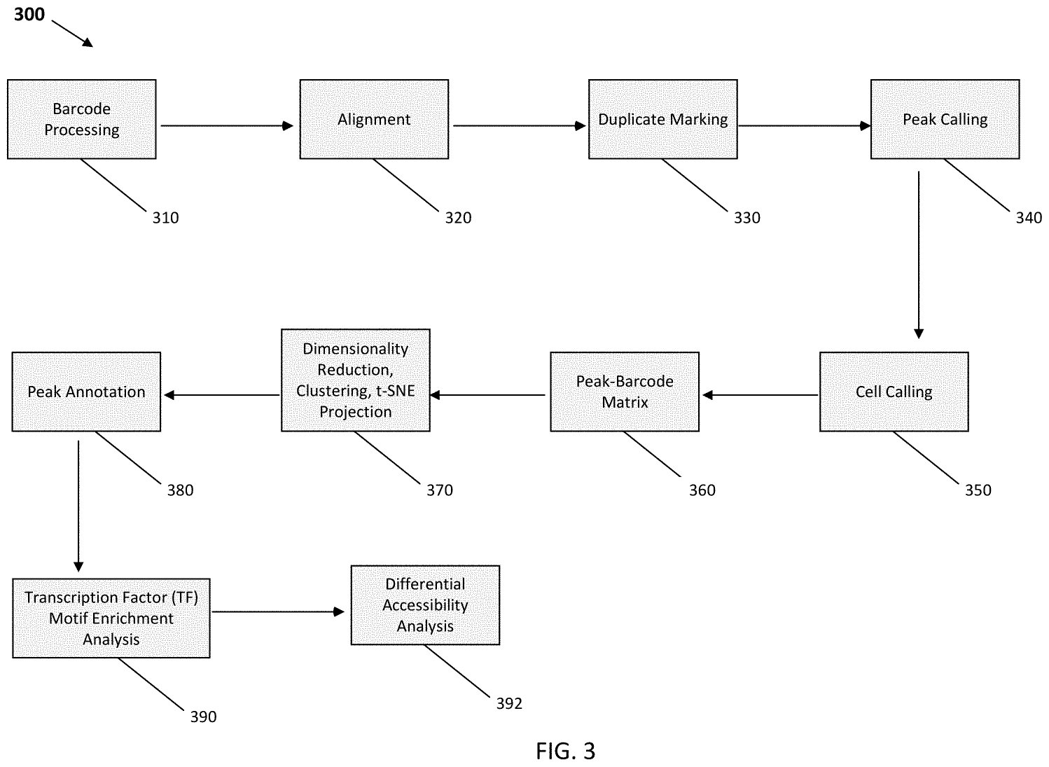

In accordance with various embodiments, a general schematic workflow is provided in FIG. 3 to illustrate a non-limiting example process of a sequencing data analysis workflow for analyzing the single cell ATAC sequencing data for identifying genome-wide differential accessibility of gene regulatory elements. The workflow can include various combinations of features, whether it be more or less features than that illustrated in FIG. 3. As such, FIG. 3 simply illustrates one example of a possible ATAC sequencing data analysis workflow.

FIG. 3 provides a schematic workflow 300, which is an expansion of the sequencing data analysis workflow 290 of FIG. 2, in accordance with various embodiments. It should be appreciated that the methodologies described in the workflow 300 of FIG. 3 and accompanying disclosure can be implemented independently of the methodologies for generating single cell ATAC sequencing data described in FIG. 2. Therefore, FIG. 3 can be implemented independently of the sequencing data generating workflow as long as it is capable of sufficiently analyzing single cell ATAC sequencing data for identifying genome-wide differential accessibility of gene regulatory elements in accordance with various embodiments.

Accordingly, in accordance with various embodiments, systems and methods within the disclosure can further include a data analysis workflow (i.e., a computational pipeline) for analyzing the sequencing data generated by the single cell ATAC sequencing workflow described above. The data analysis workflow can include one or more of the following analysis steps. Not all the steps within the disclosure of FIG. 3 need to be utilized as a group. Therefore, some of the steps within FIG. 3 are capable of independently performing the necessary data analysis as part of the various embodiments disclosed herein. Accordingly, it is understood that, certain steps within the disclosure can be used either independently or in combination with other steps within the disclosure, while certain other steps within the disclosure can only be used in combination with certain other steps within the disclosure. Further, one or more of the steps or filters described below, presumably defaulted to be utilized as part of the computational pipeline for analyzing the single cell ATAC sequencing data, can also not be utilized per user input. It is understood that the reverse is also contemplated. It is further understood that additional steps for analyzing the sequencing data generated by the single cell ATAC sequencing workflow are also contemplated as part of the computational pipeline within the disclosure.

Barcode Processing

The workflow 300 can comprise, at step 310, processing the barcodes in the single cell ATAC sequencing data for fixing the occasional sequencing error in the barcodes so that the sequenced fragments can be associated with the original barcodes, thus improving the data quality. Detail related to the barcode processing and correction as part of the various embodiments disclosed herein is provided below.

In accordance with various embodiments, the barcode sequence can be between about 2 bp to about 25 bp. In accordance with various other embodiments, the barcode sequence can be between about 5 bp and 20 bp. In accordance with various preferred embodiments, the barcode sequence can be between about 10 bp and 16 bp. The length of the barcode sequence can affect the number of unique barcodes present in the sequencing library. Accordingly, it is understood that barcode sequences shorter than 10 bp can be selected in accordance with various embodiments herein, provided that the read sequence data from multiple cells are not associated with the same barcode because of severe lack of diversity caused by a shorter length of the barcode sequence. The barcode sequence can be obtained from the “I2” index read and is read as part of the I2 reaction. Accordingly, it is understood that barcode sequences longer than 16 bp can be selected in accordance with various embodiments herein, provided that the barcode sequence length is within the limits of the I2 index read and reaction, and that it can be sequenced on a sequencer within the various embodiments herein. The barcode processing step can include checking each barcode sequence against a “whitelist” of correct barcode sequences. The barcode processing step can further include counting the frequency of each whitelist barcode. The barcode processing step can also include various barcode correction steps as part of the various embodiments disclosed herein. Non-whitelist barcodes may be corrected by identifying the minimum distance whitelist barcode using metric distances such as Hamming or Levenshtein distances. For example, one may attempt to correct the barcodes that are not included on the whitelist by finding all the whitelisted barcodes that are within 1, 2, or 3 differences (Hamming distance or Levenshtein distance) of the observed sequence, and then scoring them based on the abundance of that barcode in the read data and quality value of the incorrect bases. As another example, an observed barcode that is not present in the whitelist can be corrected to a whitelist barcode if it has >90% probability of being the real barcode bases.

Alignment

The workflow 300 can comprise, at step 320, aligning the read sequences (also referred to as the “reads”) to a reference sequence. One of more sub-steps can be utilized for trimming off adapter sequences, primer oligo sequences, or both in the read sequence before the read sequence is aligned to the reference genome. Detail related to trimming and aligning the read sequences as part of the various embodiments disclosed herein is provided below.

In the alignment step of the various embodiments herein, a reference-based analysis is performed by aligning the read sequences (also referred to as the “reads”) to a reference sequence. The reference sequence of the various embodiments herein can include a reference genome sequence and its associated genome annotation, which includes gene and transcript coordinates. The genome sequences and annotations (e.g., gene and transcript coordinates) of various embodiments herein can be obtained from reputable, well-established consortia, including but not limited to, NCBI, GENCODE, Ensembl, and ENCODE. In various embodiments, the reference sequence can include single species and/or multi-species reference sequences. In various embodiments, systems and methods within the disclosure can also provide pre-built single and multi-species reference sequences. In various embodiments, the pre-built reference sequences can include information and files related to regulatory regions including, but not limited to, annotation of promoter, enhancer, CTCF binding sites, and DNase hypersensitivity sites. In various embodiments, systems and methods within the disclosure can also provide building custom reference sequences that are not pre-built.

The alignment step may further include various sub-steps. For example, in one embodiment within the disclosure, the alignment step may include a sub-step that trims off the adapter, primer oligo sequence, or both in the read sequence before the read sequence is aligned to the reference genome. In another embodiment within the disclosure, the 3′ end of a read (i.e., the end of the read sequence) is presumed to contain the reverse complement of the primer sequence if the read sequence length is greater than the length of the genomic fragment. In such an event, the alignment step within various embodiments herein may include a sub-step that can identify the reverse complement of the primer sequence at the end of each read and trims off such sequence from the read sequence before the read sequence is aligned to the reference genome. In one embodiment within the disclosure, the cutadapt tool can be used to identify and trim off the reverse complement of the primer sequence from the read sequence prior to alignment.

The trimmed read-pairs described above can then be aligned to a specified genome reference using various methods in accordance with various embodiments herein. For example, in one embodiment within the disclosure, BWA-MEM with default parameters can be used to align all the trimmed read-pairs to a specified reference. In various embodiments, such default parameter can be bwa mem −t<num_threads>−M −R<readgroup_header><ref_fasta><R1.fastq><R2.fastq>. BWA-MEM does not align read sequences that are less than 25 bp. Accordingly, when BWA-MEM is used with various embodiments herein, the unaligned reads are included in the BAM output and flagged as unmapped. In various embodiments, the unmapped sequences may not be used in downstream analysis as they may be incapable of contributing to any information due to lack of mapping.

Duplicate Marking

The workflow 300 can comprise, at step 330, marking sequencing and PCR duplicates and outputting high quality de-duplicated fragments. One or more sub-steps can be employed for identifying duplicate reads, such as sorting aligned reads by 5′ position to account for transposition event and identifying groups of read-pairs and original read-pair. The process may further include filters that, when activated in various embodiments herein, can determine whether a fragment is mapped with MAPQ>30 on both reads (i.e., includes a barcode overlap for reads with mapping quality below 30), not mitochondrial, and not chimerically mapped. Detail related to duplicate marking as part of the various embodiments disclosed herein is provided below.

A barcoded fragment may get sequenced multiple times due to the PCR amplification process of the various embodiments herein, e.g., the ATAC sequencing workflow described above. One may mark such duplicates in order to identify the original fragments that constitute the library and actually contribute to the complexity of the library. Duplicate-forming processes can happen in an exemplary process: linear amplification, which can be done in the GEM partitions, produces copies of each fragment and is followed by PCR after emulsion breaking. De-duplication can be done with or without the barcode sequence as an independent key. For example, discarding the barcode sequence can improve accuracy by reducing the impact of barcode exchange during PCR amplification. Keeping the barcode sequence can improve sensitivity. Various methods, in accordance with various embodiments herein, can be employed to find duplicate reads. In one embodiment within the disclosure, duplicate reads are found by identifying groups of read-pairs, across all barcodes, where the 5′ ends of both R1 and R2 reads have identical mapping positions on the reference. In this embodiment, R1 and R2 refer to Read 1 and Read 2, respectively, and are standard Illumina sequencing primer sites that are used in paired-end sequencing methods. This process of identifying groups of read-pairs presumably corrects for soft-clipping of reads, which essentially means that the sequence portions of the read that do not match the reference genome on either side of the read are ignored for the sequence alignment. These identified groups of read-pairs is presumed to arise from the same original fragment. With the identified groups of read-pairs in hand, the most common barcode sequence is identified among each group of read-pairs, and the read-pairs with that barcode sequence is labelled as the “original” while the other read-pairs in the group of read-pairs are marked as duplicates of that fragment in the BAM file.

While processing the group of identically aligned read-pairs as described above, once the original fragment is marked, the systems and methods within the disclosure may further include filters that, when activated by default in various embodiments, can determine whether the fragment is mapped with MAPQ>30 on both reads (i.e., includes a barcode overlap for reads with mapping quality below 30), not mitochondrial, and not chimerically mapped. If the fragment passes these filters (i.e., the fragment is mapped with MAPQ>30 on both reads, not mitochondrial, and not chimerically mapped), this is the only read-pair entry that is created as a fragment in the fragment file (e.g., the fragments.tsv.gz file) and the start and end of the fragment is marked after adjusting the 5′ ends of the read-pair to account for the transposition event (during which the transposase occupies a region of DNA 9 base pairs long) described above in the ATAC sequencing workflow.

With this read-pair entry in hand, one or more associations can be made within the disclosure. For example, in one embodiment within the disclosure, the entry is marked as the most common barcode sequence observed for the group of read-pairs, and the number of times this fragment is observed in the library is referred to as size of the group. It is to be noted that as a consequence of this approach of the various embodiments herein, each unique interval on the genome can be associated with one or more barcodes. Each read-pair entry of the various embodiments herein can be tab-separated and the file can be position-sorted and then outputted as high quality de-duplicated fragments. In one embodiment within the disclosure, the tab-separated and position-sorted entry can be run through a generic indexer for TAB-delimited genome position files (e.g., the SAMtools tabix) with default parameters.

Peak Calling

The workflow 300 can comprise, at step 340, a peak calling analysis that includes counting cut sites in a window around each base-pair of the genome and thresholding it to find regions enriched for open chromatin. Peaks are regions in the genome enriched for accessibility to transposase enzymes. Detail related to peak calling as part of the various embodiments disclosed herein is provided below.

Only open chromatin regions that are not bound by nucleosomes and regulatory DNA-binding proteins (e.g., transcription factors) are accessible by transposase enzymes for ATAC sequencing. Therefore, the ends of each sequenced fragment of the various embodiments herein can be considered to be indicative of a region of open chromatin. Accordingly, the combined signal from these fragments can be analyzed in accordance with various embodiments herein to determine regions of the genome enriched for open chromatin, and thereby, to understand the regulatory and functional significance of such regions. Therefore, using the sites as determined by the ends of the fragments in the position-sorted fragment file (e.g., the fragments.tsv.gz file) described above, the number of transposition events at each base-pair along the genome can be counted. In one embodiment within the disclosure, the cut sites in a window around each base-pair of the genome is counted. In a further embodiment within the disclosure, a smoothed profile of the transposition events with a 401 bp moving window sum around each base-pair can be generated and can be fit to a mixture model (e.g., a Zero-Inflated Negative Binomial Algorithm (ZIMBA)-like mixture model) consisting of a geometric distribution to model zero-inflated count, negative binomial distribution to model noise, and additionally, another geometric distribution to model the signal.

In accordance with various embodiments herein, the fitting step can be performed in a way to ensure a fixed ordering of the means of mixture components. For example, in one embodiment, the means of mixture component first includes the negative binomial noise mean, followed by a geometric zero-inflation mean, and finally by the geometric signal distribution mean. In accordance with various embodiments herein, a signal threshold is set. For example, the signal threshold can be set based on an odds-ratio of ⅕ that determines, at base pair resolution, whether a region is a true peak signal (i.e., enriched for open chromatin) or represents a noise. Consequently, it can be understood that not all cut sites are within a peak region. In accordance with various embodiments herein, peaks within 500 bp of each other can be merged to produce a position-sorted file (e.g., a BED file) of peaks.

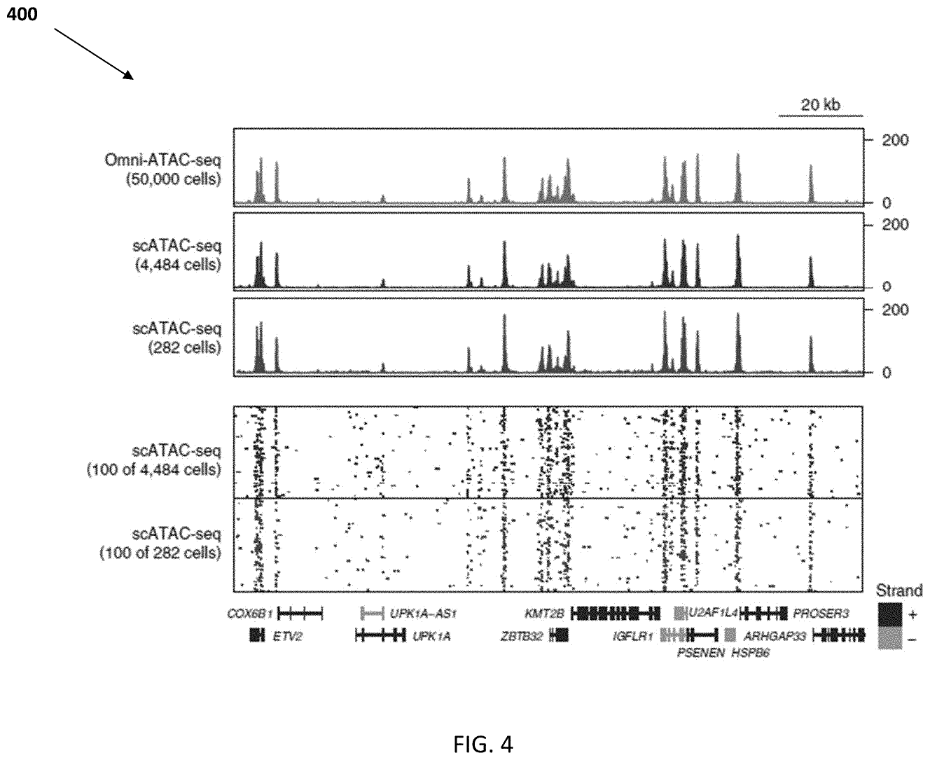

FIG. 4 (Satpathy AT., et al., Nat Biotechnol. 2019 August; 37(8):925-936) is an illustration 400 that shows exemplary genome tracks and peaks from analysis of single cell ATAC sequencing profiles, in accordance with various embodiments. Genome tracks of scATAC-seq profiles of different cell types are shown. The bottom panel shows accessibility profiles (peaks) of 100 random cells from each experiment. Each pixel represents a 100-bp region. In accordance with various embodiments herein, one or more peaks are defined by the aligned fragment sequence reads, where each peak represents an enrichment of the aligned fragment sequence reads at a given position on the reference sequence.

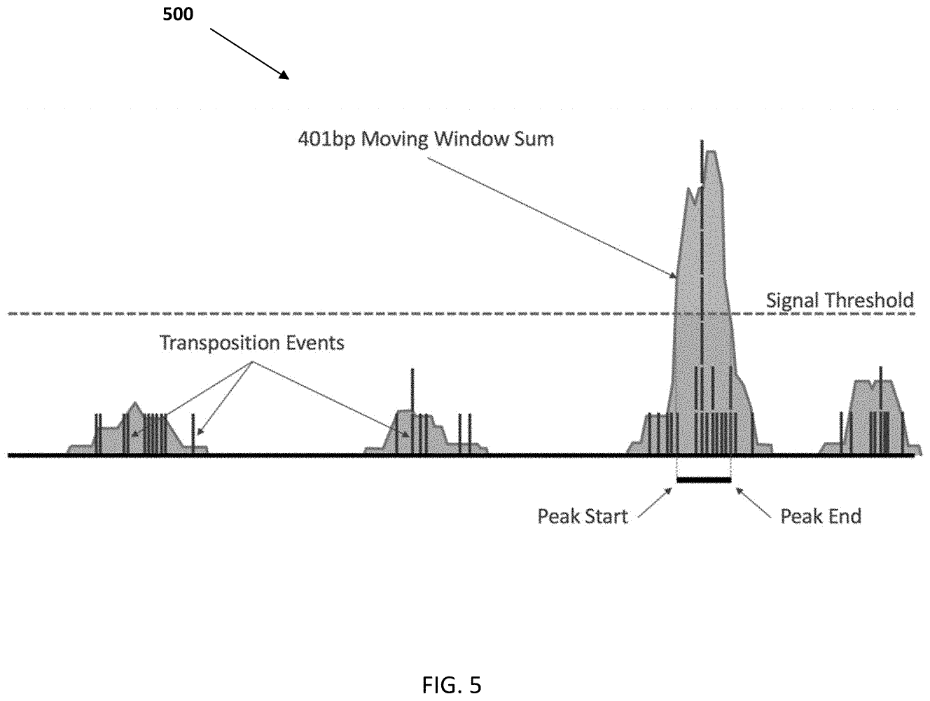

FIG. 5 is a plot 500 that illustrates how transposition events are measured with a specific 401 bp moving window sum around each base-pair along the genome, in accordance with various embodiments disclosed herein. In general, pileups of the cut sites on the genome can exhibit mountain-peaks like profile, i.e., peaks of cut sites in the midst of general, random background signal. Thus, barcodes can produce a random background signal below a pre-set signal threshold instead of exhibiting an identified a “peak”, which signify a putatively important region of the genome. In various embodiments, a peak region can be between about 500 kilo base pairs (kb) to about 5 kb in length and the background region can be either genome-wide (i.e., can be between about 2 giga base pairs (gb) to about 3 gb) or can be within a large region of the chromatin (i.e., can be between about 100 kb to 1 mega base pairs (bp)).

In accordance with come embodiments herein, the window size, the odds-ratio, and the distance to merge peaks disclosed herein were selected to maximize the area under the receiver-operator curve when the peaks are compared to a high-quality list of DNase hypersensitive sites for GM12878 from ENCODE, to produce satisfactory clustering metrics, as well as cell type identification in a set of peripheral blood mononuclear cells (PBMC) libraries. It is understood that other window sizes, odds-ratios, and distances to merge peaks in addition to the ones disclosed herein can be selected in accordance with various embodiments herein. In various embodiments, the moving window sum around each base-pair can be at least 50 bp. In various embodiments, the moving window sum around each base-pair can be at least 401 bp. In various embodiments, the moving window sum around each base-pair can be at least 50 bp, at least 100 bp, at least 200 bp, at least 300 bp, at least 400 bp, at least 500 bp, at least 600 bp, at least 700 bp, at least 800 bp, at least 900 bp, at least 1000 bp, at least 1200 bp, or at least 1500 bp. In various embodiments, the signal threshold can be set based on an odds-ratio of at least 0.2. In various embodiments, the odds-ratio can be between about 0.001 (1E⁻³) and about 1000 (1E⁻³). In various embodiments, peaks within at least 500 bp of each other can be merged. In various embodiments, peaks within at least 50 bp, at least 100 bp, at least 200 bp, at least 300 bp, at least 400 bp, at least 500 bp, at least 600 bp, at least 700 bp, at least 800 bp, at least 900 bp, at least 1000 bp, at least 1200 bp, or at least 1500 bp, of each other can be merged. It is further understood that this method of identifying peaks according to one embodiment within the disclosure is independent of the barcodes and their cellular (or non-cell) identity, which allows for inclusion of the signal from all real genomic fragments as determined by mapping. A theoretical disadvantage of such a method may be not being able to identify rare peaks appearing only in very rare populations.

Peak caller programs in the public domain essentially all face the general problem of thresholding the difference between background noise and signal (i.e. peak), for example, in the genomic distribution of Tn5 cut sites in single cell assay for transposase accessible chromatin (scATAC) data. Model-based Analysis of ChIP-seq (MACS2), for example, does single distribution fitting to a Poisson, which is not very accurate for most data. Other peak callers can lack a fully optimized mixture model distribution fitting, leading to poorly-fitting local optima.

In accordance with various embodiments, a method is provided for generating peak calls for a genomic data set (or barcode genomic sequence dataset). This improved peak caller method can provide, for example, a more robust mixture model distribution fitting, leading to a more accurate and robust global threshold across the entire data set under consideration. The method can comprise receiving a genomic data set and generating a signal track on the genome for the members of the data set. For example, the signal track can include determining a cumulative signal at each position across a constant, but moving, window. This window can vary as needed but can be, for example a constant length of about 400 bp (see above for reference to a 401 bp moving window). Such a constant length window can allow for an overall smoothing of the observed signal.

The method can further comprise applying an initial thresholding on the signal track (also referred to as a global thresholding). This threshold can be set based on count values whereby any count value above the established threshold (higher count data) possesses sufficient signal to be considered a peak for peak calling purposes. Such an initial thresholding at least allows for calling of larger sized peaks (e.g., about 20 kb long).

It should be noted that, in many circumstances, there can exist local variation in the background noise between genomic regions, which can lead to missed peak calls or false peak calls due to the varied contribution of noise to those different regions.

In accordance with various embodiments, the method can further comprise applying a multi-component mixture model to the data set. The multi-component mixture model can be a 3-component mixture model. At least one of the components can be a discrete probability distribution. The components can be selected from the group consisting of a zero-inflated noise component, a negative binomial noise component, and a combination thereof. For example, a 3-component mixture model can include a zero-inflated noise component (e.g., with a geometric distribution), a negative binomial noise component for the general noise, and a negative binomial noise component (for signal). Moreover, an expectation maximization algorithm can be initiated and converged toward a local optima.

The method can further comprise generating a global threshold, wherein the threshold can be a fixed tail probability of the noise components of the multi-component mixture model.

In accordance with various embodiments, a method is provided for generating peak calls for a genomic data set (or barcode genomic sequence dataset). The method can include masking out a portion of the genomic data set (e.g., genomic data having the highest counts) using a masking threshold value to produce a subset of the genomic data set for further analysis. In accordance with various embodiments, the masking threshold can be between about 5% and 50% whereby between the top 95% of counts (for a 5% threshold) and top 50% of counts are removed from the data set such that the remaining portion of the data is included for further downstream processing. In accordance with various embodiments, the masking threshold can be between about 5% to 100%, 10% to 15%, 15% to 20%, 20% to 25%, 25% to 30%, 35% to 40%, 40% to 45%, 45% to 50%, and any other contemplated range utilizing these given values. By masking out high counts from the genomic data set to generate the subset, further analysis of the genomic data set can be constrained to those signals (i.e., the subset) that can be most affected by noise, as the high-count data is more likely to be unambiguous signal data.

The method can further include determining, for the subset, a first maximum likelihood fitting of a first discrete probability distribution. The first discrete probability distribution can include, for example, a zero-inflated noise component with a single geometric distribution. This fitting can be ideal when applied to the lower signal subset of the genomic data set. The fitting can also result in residual signal exceeding the lower signal fit to the distribution. To fit such residual signal, an additional component may be implemented to fit the residual signal.

The method can therefore further include identifying a first residual signal data set from the first maximum likelihood fitting. The identifying of the first residual signal data set can include generating weighted counts for the subset based on the determined first maximum likelihood fitting. The identifying of the first residual signal data set can further include extracting data exceeding the fit. In accordance with various embodiments, identified first residual signal data can undergo further processing as discussed below.

The method can further include determining a second maximum likelihood fitting of a second discrete probability distribution. The second discrete probability distribution can include, for example, a first negative binomial noise component for the residual signal data from the subset.

The method can further include applying a first expectation maximization on at least a truncated data set from the subset to fit the truncated data set to background noise data. The first expectation maximization can include employing a mixture model (e.g., a 2-component mixture model) on the truncated data set. In accordance with various embodiments, the truncated data set can be generated from the from the first and second maximum likelihood fittings discussed above. This step can also include iterating to converge the fit of the truncated data to the background data.

The method can further include applying a second expectation maximization on the full data set to generate a final distribution. Applying the second expectation maximization can include employing a mixture model (e.g., a 2-component mixture model) on the full data set. Applying the second expectation maximization can include capturing a second residual signal data set from the full data set. Applying the second expectation maximization can include fitting a third discrete probability distribution (e.g., second negative binomial component) to the second residual signal data set. Applying the second expectation maximization can further include initializing the second expectation maximization with a 3-component mixture model, which would also include the third discrete probability distribution (e.g., second negative binomial component). Applying the second expectation maximization can further include converging the second expectation maximization to a final distribution.

The method can further include generating a final threshold as a function of the first and second discrete probability components (e.g., zero-inflated noise component with a single geometric distribution and first negative binomial noise component for the residual signal data from the subset). The final threshold can be, for example, a fixed tail probability of the two noise components. For example, a variable tail q-value can be used to set the final threshold. The variable q-value can be, for example, 5%, but can be manipulated as needed in view of the data set under analysis.

The method can further include applying the final (or global) threshold across the full data set and making peak calls accordingly. It should be noted that, in accordance with various embodiments, various types discrete probability distributions can be employed for this improved peak calling methods, the type of probability distribution contemplated should not be limited to the various discrete probability distributions discussed herein.

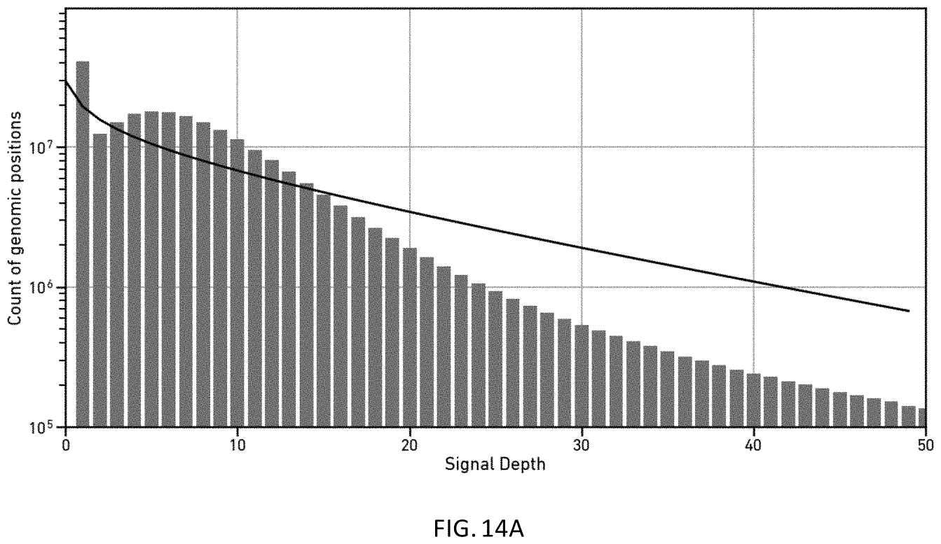

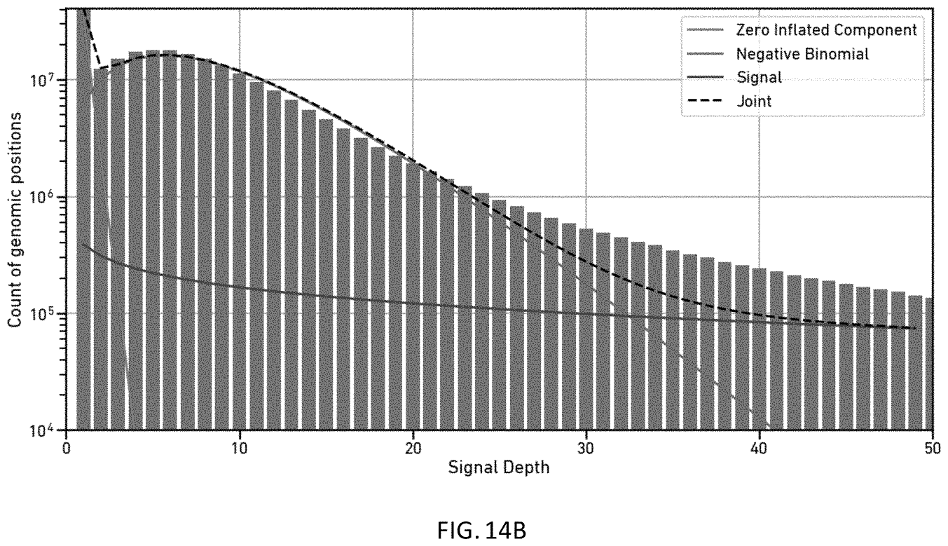

Referring to FIG. 14, two charts are provided that establish the improvement in peak calling versus publicly available methods. In FIG. 14A, the line chart describes the association of count of genomic positions as a function of signal depth on a data set subject to a single negative binomial noise component fit, where the threshold was set to 50 and q-value set to 5%. In FIG. 14B, the line chart describes the association of count of genomic positions as a function of signal depth on a data set subject to the modified mixed model methodology discussed in accordance with various embodiments herein. As is apparent when comparing the solid line on FIG. 14A (representing the overall fit) to the dotted line on FIG. 14VB (also representing the overall fit), that the line illustrated on FIG. 14B (via the improved methods) is much more accurate than the fit line illustrated in FIG. 14A (representing public domain methods).

It has also been observed that for small regions of the genome (e.g., ˜tens of kb), it becomes difficult to identify regions of systematically enriched signal, or peaks in those small regions, using current peak calling tools. One example is with scATAC signal, which can have some shape commonalities such as, for example, peaks that have a fairly narrow true width of less than ˜2 kb and at least ˜100 bp. Various currently available peak calling tools use either a constant fixed signal threshold or a locally variable signal threshold (e.g., MACS2) that is dependent on the local signal distribution. For candidate regions larger than a single peak, these tools may inadequately measure or account for local behavior. Moreover, local signal distribution generally depends on the peak density, which can mean that a high density of peaks in the region can raise the local threshold for peak calling in most approaches, thereby reducing the number of true peaks identified.

As a result, current methods can mask true peaks due for many reasons. For example, current methods can merge multiple small peaks into one peak signal, leading to one called peak (which is not an accurate call) and also various missed peak calls. Also, with multiple peaks within a region, current methods allow the total signal from the multiple peaks to lift the threshold to a level that masks peaks, leading to missed peak calls. Moreover, it becomes difficult to separate individual smaller peaks from regions with high elevated BR noise using current methods. Even for current methods that implement a local threshold (e.g., MACS2), that local thresholding is conducted at specific regions using generally less effective distribution such as a Poisson distribution. Therefore, for regions with heightened noise, the noise will lift the local threshold, masking over smaller peaks, thus leading to missed peak calls.

In accordance with various embodiments, a method is provided for generating peak calls for a genomic data set (or barcode genomic sequence dataset). The method accounts for small regions on the genome by providing local thresholds within a given region to improve the accuracy of peak calls within those small regions. The small regions can range from about 1 to about 100 kb, or any combination of values within that given range.

The method can include performing a wavelet transform on a local signal with a fixed wavelet width. The fixed width can be, for example, a width between about 50 bp to about 2000 bp. The width can be, for example, about 300 bp). Such fixed wavelet width can allow for tracking with expected scATAC peak sizes. The wavelet transform can be, for example, the Ricker (Mexican hat) wavelet, though the type of wavelet transform can vary and should not be limited to those types provided herein.

The method can further include identifying the local maxima of the wavelet transformed signal as a putative peak(s).

The method can further include bounding the putative peaks at a percentage of their maximal prominence. The percentage can be, for example, a range of about 50% to about 95% and any other range within that about 50% to about 95% range. The percentage can be, for example, 95% of their maximal prominence.

The method can further comprise performing a loop through each putative peak region. The loop can include calculating a first median real (as opposed to wavelet-transformed) signal inside the putative peak region as the peak signal. The loop can further include calculating a second median real signal nearby the putative peak region but outside all other putative peaks. For example, the second median signal nearby can include a region width that is dependent on the size of putative peak under examination. For example, the width can be 1×, 3× or 5× the peak width. Moreover, the calculation of the second median real signal can include examining one or more widths around the putative peak. By observing the nearby region that is outside other putative peaks, one can avoid the current problem of allowing outside signals to artificially pull up the local threshold and mask true peaks.

The loop can further include calculating a beta distribution with a given prior and given Bayesian update. For example, for a prior of (2,2) and Bayesian update of (peak_signal, local_noise), the beta distribution would represent the estimated distribution of the ratio of peak signal to local noise. The loop can further include determining that, for a beta distribution and a local threshold, that if at least a given percentage of the probability mass of the beta distribution is above the local threshold, then the putative peak in question is reported as a true peak. For example, for a given beta distribution, it the distribution has at least, e.g., 95% of its probability mass above a local threshold of, e.g., 0.6, the putative peak in question would be reported as a true peak. This local threshold can be a function of a given signal-to-noise threshold (SNRT) such that the local threshold can be SNRT/(1+SNRT). For example, given an SNRT of 1.5, the local threshold would be 1.5/(1+1.5), or 0.6, as provided above.

In accordance with various embodiments, a method is provided for generating peak calls from a barcode genomic sequence dataset, the generating of peak calls comprising determining a cumulative signal at each position along a signal track of the genome across a constant, moving window. The method further comprises applying an initial threshold on the signal track, applying a multi-component mixture model to the dataset, wherein an expectation maximum is initiated on the data set and converged to a final distribution, generating a global threshold as a probability of the components of the multi-component mixture model and applying the global threshold across the full data set for making peak calls.

The method can further comprise applying a masking threshold on a portion of the dataset, wherein the threshold is a percentage value, wherein all counts above the threshold are masked and all counts below the threshold are compiled into a data subset. The method can further comprise determining a first maximum likelihood fitting of a first discrete probability distribution for the data subset and identifying a first residual signal data set from the first maximum likelihood fitting. The method can further comprise determining a second maximum likelihood fitting of a second discrete probability distribution on the first residual data set. The method can further comprise generating a truncated data set from at least one of the first and second likelihood fittings, and applying a first expectation maximization on the truncated data set to fit the truncated data set to background noise data. The method can further comprise applying a second expectation maximization on the full data set to generate a final distribution, and generating the global threshold as a function of the first and second discrete probability distributions.

The method can further comprise performing a wavelet transform on a local signal within the data to produce a wavelet transformed signal, identifying a putative peak as a local maxima of the wavelet transformed signal and bounding the putative peak at a maximal prominence percentage of the putative peak. The method can further comprise performing a loop through a putative peak region, including calculating a beta distribution across the putative peak region, determining a local threshold as a function of signal to noise ratio of the putative peak region, and calling the putative peak as a true peak when a probability mass percentage of the beta distribution is above the local threshold.

In accordance with various embodiments, the constant, moving window can have a length of about 400 bp.

In accordance with various embodiments, the multi-component mixture model can be a 3-component mixture model, wherein the components can be discrete probability distributions selected from the group consisting of a zero-inflated noise component, a negative binomial noise component, and a combination thereof.

In accordance with various embodiments, the global threshold can be a fixed tail probability of the components of the multi-component mixture model.

In accordance with various embodiments, the masking threshold can be between about 5% to about 50%.

In accordance with various embodiments, the first discrete probability distribution can be a zero-inflated noise component with a single geometric distribution.

In accordance with various embodiments, the identifying of the first residual signal data set comprises: generating weighted counts for the subset based on the determined first maximum likelihood fitting, and extracting data exceeding the fit as the first residual signal data set.

In accordance with various embodiments, the second discrete probability distribution can be a first negative binomial noise component.

In accordance with various embodiments, the first expectation maximization can include applying a 2-component mixture model on the truncated data set.

In accordance with various embodiments, the second expectation maximization can include applying a 3-component mixture model on the truncated data set.

In accordance with various embodiments, the method further comprises performing a wavelet transform on the local signal, with a fixed wavelet width, within the data to produce a wavelet transformed signal. In accordance with various embodiments, the fixed wavelet width is between about 50 bp to about 2000 bp. In accordance with various embodiments, the fixed wavelet width is about 300 bp.

In accordance with various embodiments, the maximal prominence percentage is between about 50% and about 95%. In accordance with various embodiments, the maximal prominence percentage is 95%.

In accordance with various embodiments, performing a loop through the putative peak region comprises: calculating a first median real signal inside the putative peak region as the peak signal; calculating a second median real signal at a given width in the putative peak region but outside all other observed putative peaks; and calculating the beta distribution with a prior and a Bayesian update.

In accordance with various embodiments, the width can be one or more widths, and the width is selected from the group consisting of 1× the peak width, 3× the peak width, 5× the peak width, and combinations thereof.

In accordance with various embodiments, the probability mass percentage is at least about 95%.

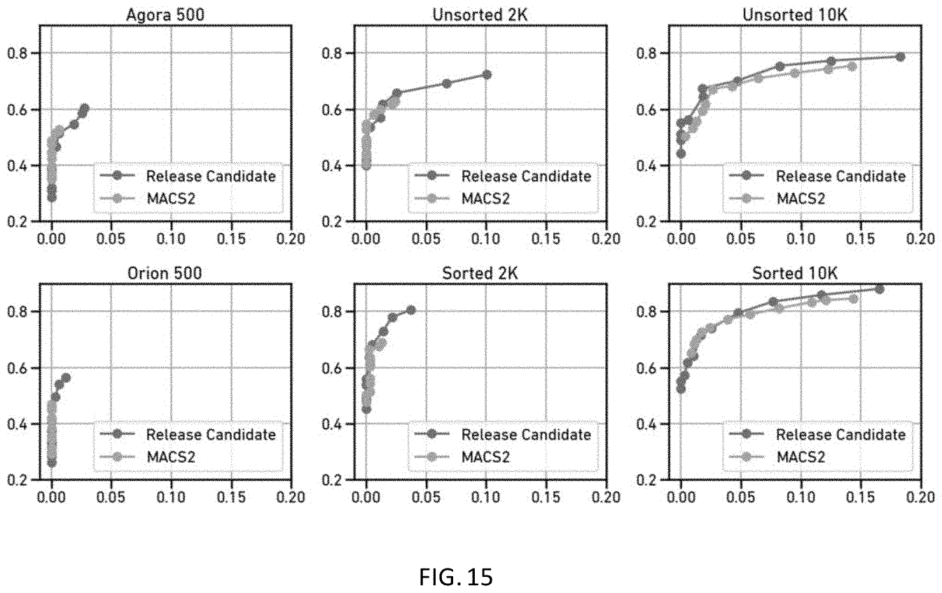

Referring to FIG. 15, multiple charts are provided illustrating receiver-operator characteristic (ROC) curves (e.g., false positive rate on the X-axis vs. the true positive rate on the Y-axis) comparing the new peak calling method (“Release Candidate”), in accordance with the various methods discussed herein, to a currently available method for peak calling (“MACS2”). Each subplot shows performance on a different sample. Each point represents the accuracy and precision of a single set of peak calls by one tool with the aggressiveness (e.g., q-value parameter) tuned to different levels. Lower left is more conservative, upper right is more aggressive. It is apparent by comparing the two methods that the “Release Candidate” performs uniformly better than MACS2, as at any false positive rate it has a higher true positive rate.

Cell Calling

The workflow 300 can comprise, at step 350, a cell calling analysis that includes associating a subset of barcodes observed in the library to the cells loaded from the sample. Identification of these cell barcodes can allow one to then analyze the variation in data at a single cell resolution. The process may further include correction of gel bead artifacts, such as gel bead multiples (where a cell shares more than one barcoded gel bead) and barcode multiplets (which occurs when a cell associated gel bead has more than one barcode). In some embodiments, the steps associated with cell calling and correction of gel bead artifacts are utilized together for performing the necessary analysis as part of the various embodiments herein.

In accordance with various embodiments, the record of mapped high-quality fragments that passed all the filters of the various embodiments disclosed in the steps above and were indicated as a fragment in the fragment file (e.g., the fragments.tsv file), are recorded. With the peaks determined in the peak calling step disclosed herein, the number of fragments that overlap any peak regions, for each barcode, can be utilized to separate the signal from noise, i.e., to separate barcodes associated with cells from non-cell barcodes. It is to be understood that such method of separation of signal from noise works better in practice as compared to naively using the number of fragments per barcode.

Various methods, in accordance with various embodiments herein, can be employed to for cell calling. In one embodiment within the disclosure, the cell calling can be performed in two steps. In the first step of cell calling of the various embodiments herein, the barcodes that have fraction of fragments overlapping called peaks lower than the fraction of genome in peaks are identified. When this first step is employed in the cell calling process of the various embodiments herein, the peaks are padded by 2000 bp on both sides so as to account for the fragment length for this calculation. It has been observed that these barcodes typically have their cut sites randomly distributed over the genome, are not targeted to be enriched near functional regions, and do not exhibit the expected ATAC-seq “peaky” signal. As used herein, a peaky signal refers to a region in the genome with enriched signal compared to the background. Accordingly, this step of the various embodiments herein, can be used to mask the ‘low targeting’ barcodes lacking the ATAC signal out of the total set of barcodes observed for the library prior to cell calling.

Correction of Gel Bead Artifacts

Various methods, in accordance with various embodiments herein, are employed for correction of gel bead artifacts. Systems and methods of the various embodiments herein, e.g., the 10× Chromium system, is observed to have a low rate of gel bead multiplets. Gel bead multiplets can be understood to arise where a cell shares more than one barcoded gel bead. In one embodiment within the disclosure herein, such multiples are observed to be predominantly doublets where a cell shares two barcoded get beads. These cells can be manifested as multiple barcodes of the same cell type in the dataset. The presence of these few extra barcodes presumably do not affect the secondary analysis of the various embodiments herein, such as clustering or differential analysis, although it can potentially inflate abundance measurements of very rare cell types.

The sequencing data of the various embodiments herein, e.g., the single cell ATAC data, may have another potential source of generating artifacts with extra cells of similar kind. This phenomenon is known as barcode multiplets, which can be understood to occur when a cell associated gel bead is not monoclonal and has the presence of more than one barcode.

With the correction of gel bead artifacts, the second step of cell calling of the various embodiments herein, can be performed on the remainder barcodes. In embodiment of with the disclosure, a depth-dependent fixed count can be subtracted from all barcode counts to model the whitelist contamination. This fixed count can be understood to be the estimated number of fragments per barcode that originated from a different barcoded bead (e.g., a Gel bead-in-EMulsion (GEM)), when assuming a contamination rate of 0.02. In the next step of the various embodiments herein, the mixture model of two negative binomial distributions is fit to capture the signal and noise. Setting an odds-ratio of 100000, which appeared to work best with internal testing in accordance with various embodiments herein, the barcodes that correspond to real cells were separated from the non-cell barcodes. It is understood that odds-ratios in addition to the ones disclosed herein can be selected in accordance with various embodiments herein.

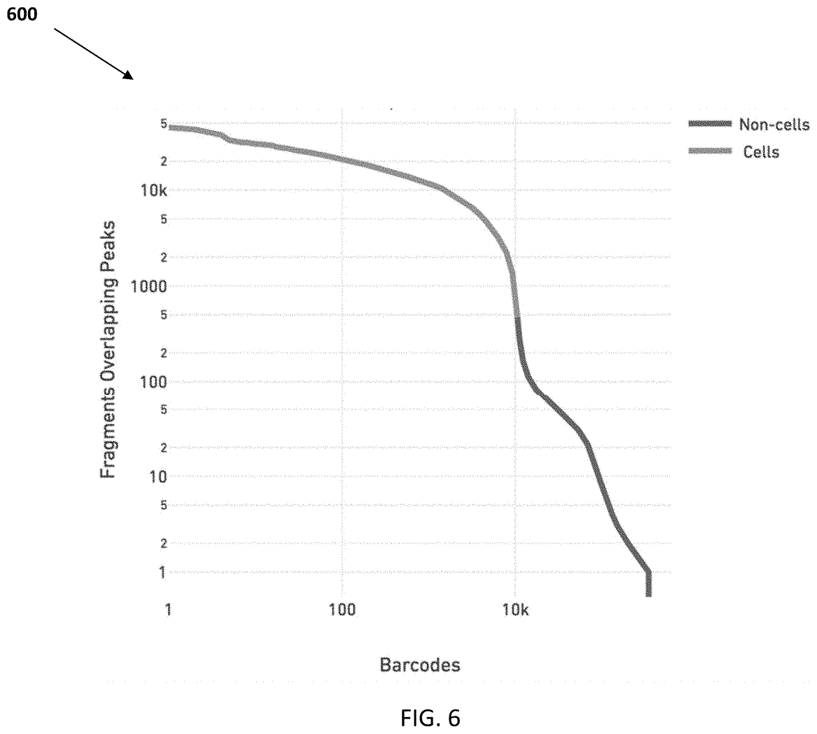

The cell calling within various embodiments herein, may be limited to produce <20 k cells per species in the reference. Such observed behavior can be understood to arise if the systems and methods of the various embodiments herein is designed to support 500-10 k cells. If one specifies “—force-cells=N” as a parameter in accordance with various embodiments herein, e.g., the Cell Ranger™ ATAC Count analysis pipeline, it can be ensured that the top N barcodes are reported as cells for each species, as indicated the barcode rank plot in FIG. 6. In one embodiment, N>20 k may not be accepted by the systems and methods within the disclosure herein. If “—force-cells” is not provided as a parameter of the various embodiments herein, in the case of mixed species sample, a second iteration can be performed where the non-cell barcodes are masked out, the same mixture model can be fitted to the two species distributions present in the cell barcodes, and the division of cell barcodes associated with each species can be refined. In general, it is to be understood that the “—force-cells” value, to be used with the various embodiments herein, should correspond to a point below the “knee” as seen on the barcode rank plot in FIG. 6. FIG. 6, referred to above, is a barcode rank plot 600 (also referred to as the “knee plot”) in accordance with various embodiments that shows the distribution of barcode counts for fragments overlapping peaks and marks the barcodes which can be inferred to be associated with cells. The y-axis represents the number of fragments overlapping peaks and the x-axis represents the number of barcodes. A steep drop-off in the plot can be understood to be indicative of good separation between the cell-associated barcodes and the barcodes associated with empty partitions or droplets.

Peak-Barcode Matrix

The workflow 300 can comprise, at step 360, determining the peak-barcode matrix. In accordance with various embodiments, at step 360, a raw peak-barcode matrix can be generated first, which is a count matrix consisting of the counts of fragment ends (or cut sites) within each peak region for each barcode. This raw peak-barcode matrix captures the enrichment of open chromatin per barcode. The raw matrix can then be filtered to consist only of cell barcodes by filtering out the non-cell barcodes from the raw peak-barcode matrix, which can then be used in the various dimensionality reduction, clustering and visualization steps of the various embodiments herein.

Dimensionality Reduction, Clustering, and T-SNE Projection

The workflow 300 can comprise, at step 370, various dimensionality reduction, clustering and t-SNE projection tools. Dimensionality reduction tools of the various embodiments herein are utilized to reduce the number of random variables under consideration by obtaining a set of principal variable. In accordance with various embodiments herein, clustering tools can be utilized to assign objects of the various embodiments herein to homogeneous groups (called clusters) while ensuring that objects in different groups are not similar. T-SNE projection tools of the various embodiments herein can include an algorithm for visualization of the data of the various embodiments herein. In accordance with various embodiments, systems and methods within the disclosure can further include dimensionality reduction, clustering and t-SNE projection tools. In some embodiments, the analysis associated with dimensionality reduction, clustering, and t-SNE projection for visualization are utilized together for performing the necessary analysis as part of the various embodiments herein. Various analysis tools for dimensionality reduction such as Principal Component Analysis (PCA), Latent Semantic Analysis (LSA), and Probabilistic Latent Semantic Analysis (PLSA), clustering, and t-SNE projection for visualization that allow one to group and compare a population of cells with another, detail related to which are provided below.

Biological discovery is often aided by visualization tools that allow one to group and compare a population of cells with another. In order to enable such discovery, various visualization methods within the various embodiments herein, e.g., visualization methods within the Cell Ranger™ ATAC Count analysis pipeline, can be employed. In various embodiments, such visualization methods can include clustering and T-distributed Stochastic Neighbor Embedding (t-SNE) projection tools.

The systems and methods within the disclosure are directed to identifying differential accessibility of gene regulatory elements in transposase accessible chromatin regions using single cell genomic sequencing technologies. As the data is sparse at single cell resolution, dimensionality reduction in accordance with various embodiments herein can be performed to cast the data into a lower dimensional space. Accordingly, dimensionality reduction within the various embodiments herein can be run on the normalized filtered peak-barcode matrix (i.e., cut site counts for cell barcodes in called peaks) to reduce the number of feature (peak) dimensions. In some embodiment within the disclosure, dimensionality reduction is performed before employing the clustering and t-SNE projection tools. It is understood that such dimensionality reduction can also provide the benefit of de-noising the data.

Various systems and methods of various embodiments herein, e.g., systems and methods of the Cell Ranger™ ATAC Count analysis pipeline, can be utilized to support dimensionality reduction. Various methods, such as Principal Component Analysis (PCA), Latent Semantic Analysis (LSA), and Probabilistic Latent Semantic Analysis (PLSA), can support dimensionality reduction in accordance with various embodiments herein. In one embodiment within the disclosure, before clustering the cells, LSA is run on the normalized filtered peak-barcode matrix to reduce the number of feature (peak) dimensions. This produces a projection of each cell onto the first N components (default N=15). Thus, in one embodiment within the disclosure, the adopted default method of dimensionality reduction is LSA. In other embodiments within the disclosure, users may alternatively choose PCA or PLSA to perform dimensionality reduction in the pipeline. Accordingly, users can specify which dimensionality reduction method to use by providing a dimensionality reduction parameter. In one embodiment within the disclosure, the dimensionality reduction parameter can be “—dim-reduce=<Method>”, which can be specified to the various embodiments herein, e.g., the Cell Ranger™ ATAC Count analysis pipeline. In accordance with various embodiments herein, each dimensionality reduction method within the disclosure can have an associated data normalization technique that is used prior to the dimensionality reduction step.

In accordance with various embodiments herein, a collection of clustering methods within the disclosure can be employed to accept the dimensionality reduced data. In one embodiment within the disclosure, an optimized implementation of the Barnes Hut TSNE algorithm can be employed to project the dimensionality reduced data into 2-D t-SNE space. In accordance with various embodiments herein, the number of dimensions can be fixed to 15. In some embodiments within the disclosure, it has been observed that fixing the number of dimensions 15 can sufficiently separate clusters visually and in a biologically meaningful way when tested on peripheral blood mononuclear cells (PBMCs). It is understood that other number of dimensions can be fixed in accordance with various embodiments herein. In various embodiments within the disclosure, the number of dimensions (i.e., dimensions of the matrix) can be fixed at a number less than the number of cell-barcodes and the number of peaks. In various embodiments, the number of dimensions can be at least 15. In various embodiments, the number of dimensions can be at least 5, at least 10, at least 15, at least 20, at least 25, at least 30, at least 35, at least 40, at least 45, at least 50, at least 55, at least 60, at least 65, at least 70, at least 75, at least 80, at least 85, at least 90, at least 95, or at least 100. It can be understood that higher numbers of dimensions can be computationally costly. Accordingly, computational costs can be determinative of the number of dimensions used. More detail regarding the various methods dimensionality reduction methods described above is provided below.

PCA

When PCA is the dimensionality reduction method of the various embodiments herein, the data is normalized to median cut site counts per barcode and log-transformed. In one embodiment, a fast, scalable and memory efficient implementation of IRLBA (Augmented, Implicitly Restarted Lanczos Bidiagonalization Algorithm) can be used that allows in-place centering and feature scaling and produces the transformed matrix along with the principal components (PC) and singular values encoding the variance explained by each PC. When PCA is the default dimensionality reduction method of the various embodiments herein, k-means clustering can be used. In one embodiment within the disclosure, k-means clustering produces 2 to 10 clusters for visualization and analysis. In another embodiment within the disclosure, a k-nearest neighbors graph-based clustering method is also provided via community detection using a modularity optimization algorithm. In one embodiment within the disclosure, the modularity optimization algorithm is louvain modularity optimization algorithm. The transformed matrix of the various embodiments herein, can be operated on by the t-SNE algorithm with default parameters (e.g., tsne_input_pcs, tsne_perplexity, tsne_theta, tsne_max_dims, tsne_max_iter, tsne_stop_lying_iter, and tsne_mom_switch_iter) and can provide 2-D coordinates for each barcode for visualization with various embodiments herein.

Below is a summary of default parameters of t-SNE algorithm in accordance with various embodiments.

| | | Default | | |
|---|---|---|---|---|
| Parameter | Type | Value | Recommended Range | Description |
| tsne_input_pcs | int | null | Cannot be set higher than the | Subset to top N |
| | | | num_comps parameter, which | principal components |
| | | | is N principal components for | for TSNE. |
| | | | LSA/PCA/PLSA. | |
| tsne_perplexity | int | 30 | 30-50 | TSNE perplexity |
| | | | | parameter. |
| tsne_theta | float | 0.5 | Between 0 and 1. | TSNE theta |
| | | | | parameter. |
| tsne_max_dims | int | 2 | 2 or 3. | Maximum number of |
| | | | | TSNE output |
| | | | | dimensions. |
| tsne_max_iter | int | 1000 | 1000-10000 | Number of total |
| | | | | TSNE iterations. |
| tsne_stop_lying_iter | int | 250 | Cannot be set higher than | Iteration at which |
| | | | tsne_max_iter. | TSNE learning rate is |
| | | | | reduced. |
| tsne_mom_switch_iter | int | 250 | Cannot be set higher than | Iteration at which |
| | | | tsne_max_iter. | TSNE momentum is |
| | | | | reduced. |

LSA

In accordance with various embodiments herein, the data can be normalized via the inverse-document frequency (idf) transformer, where each peak count can be scaled by the log of the ratio of the number of barcodes in the matrix and the number of barcodes where the peak has a non-zero count. This normalization can provide greater weight to counts in peaks that occur in fewer barcodes. In some embodiments within the disclosure, singular value decomposition (SVD) can be performed on this normalized matrix using IRLBA without scaling or centering, to produce the transformed matrix in lower dimensional space, as well as the components and the singular values signifying the importance of each component. In some embodiments within the disclosure, prior to clustering, normalization to depth can be performed by scaling each barcode data point to unit L2-norm in the lower dimensional space. It has been observed that the combination of these normalization techniques obviates the need to remove the first component in accordance with various embodiments herein. When LSA is the dimensionality reduction method of the various embodiments herein, a spherical k-means clustering can be provided that produces 2 to 10 clusters for downstream analysis. It has been observed that spherical k-means can perform better than plain k-means, by identifying clusters via k-means on L2-normalized data that can live on the spherical manifold. Accordingly, spherical k-means can be suitable to cluster large-scale datasets from aggregation runs, as described in detail below. In another embodiment within the disclosure, and similar to PCA, a graph-based clustering can be provided and visualized via t-SNE. However, similar to spherical k-means clustering, the data can be normalized to unit norm before performing graph-based clustering and t-SNE projection.

PLSA

PLSA is a special type of non-negative matrix factorization, with roots in Natural Language Processing. When PLSA is the dimensionality reduction method of the various embodiments herein, the KL-divergence between the empirically determined probability of observing a peak in a barcode and the lower rank approximation to it is minimized, via an Expectation-Maximization algorithm. In one embodiment, the data is not normalized prior to dimensionality reduction via PLSA. Similar to LSA and PCA, a transformed matrix, component vectors, and a set of values explaining the importance of each component can be produced in accordance with various embodiments herein. PLSA can offer natural interpretation of the components and the transformed matrix of the various embodiments herein. In accordance with various embodiments herein, each component can be interpreted as a hidden topic and the transformed matrix can simply be the probability of observing a barcode from a given topic (i.e., Prob(barcode|topic)). In accordance with various embodiments herein, the component vectors can be the probability of observing a peak from a given topic (i.e., (Prob(peak|topic)) and the counterpart to singular values of LSA/PCA can simply be the probability of each topic (i.e., (Prob(topic)) observed in the data of various embodiments herein. Similar to LSA, the transformed matrix for PLSA can be normalized to unit L2-norm. In one embodiment within the disclosure, spherical k-means clustering can be then be performed to produce 2 to 10 clusters. In another embodiment within the disclosure, graph-based clustering can also be performed. The transformed matrix of the various embodiments herein, can then be visualized via t-SNE. It is understood that while PLSA offers great advantages in interpretability of the lower dimensional space, it is appreciably slower than both PCA and LSA and does not scale well beyond 20 components on large datasets. To ameliorate this to some extent, in one embodiment within the disclosure, the in-house implementation of PLSA can be multithreaded (4 threads on compute cluster) and written and compiled in C++. To ensure a reasonable run time, in one embodiment within the disclosure, the algorithm can be capped at 3000 iterations in the event it does not converge first. It is understood that other number of dimensions can be fixed in accordance with various embodiments herein.

It is understood that various clustering methods including, but not limited to, K-Means clustering, affinity propagation, mean-shift, spectral clustering, Ward hierarchical clustering, agglomerative clustering, DBSCAN, OPTICS, Gaussian mixture models, Birch clustering, and k-medoids clustering, and visualization approaches can be utilized in accordance with various embodiments herein. It is understood that each clustering method may have various tradeoffs. Accordingly, in various embodiments, selection of a clustering method can be made based on whether the clusters make biological sense with known, well-studied sample types (e.g., PBMCs), i.e., whether the clusters generated using a particular clustering method make sense with validation on known biology. Below is a non-limiting summary of dimensionality reduction techniques and associated clustering and visualization approaches in accordance with various embodiments herein.

| Dimensionality | | |
|---|---|---|
| Reduction | Clustering | Visualization |
| PCA | K-means, graph-clustering | TSNE |
| LSA | Spherical k-means, graph-clustering | TSNE |
| PLSA | Spherical k-means, graph-clustering | TSNE |

Peak Annotation

The workflow 300 can comprise, at step 380, annotating the peaks by performing gene annotations and discovering transcription factor-motif matches on each peak. It is contemplated that peak annotation can be employed with subsequent differential analysis steps within various embodiments of the disclosure. Various peak annotation procedures and parameters are contemplated and are discussed in detail below.

Peaks are regions enriched for open chromatin, and thus have potential for regulatory function. It is therefore understood that observing the location of peaks with respect to genes can be insightful. Various embodiments herein, e.g., bedtools closest −D=b, can be used to associate each peak with genes based on closest transcription start sites (TSS) that are packaged within the reference. In accordance with some embodiments within the disclosure, a peak is associated with a gene if the peak is within 1000 bases upstream or 100 bases downstream of the TSS. Additionally, in accordance with some embodiments within the disclosure, genes can be associated to putative distal peaks that are much further from the TSS and are less than 100 kb upstream or downstream from the ends of the transcript. This association can be adopted by companion visualization software of the various embodiments herein, e.g., Loupe Cell Browser. In another embodiment, this association can be used to construct and visualize derived features such as promoter-sums that can pool together counts from peaks associated with a gene.

Peaks are also enriched for transcription factor (TF) binding motifs, and the presence of certain motifs can be indicative of transcription factor activity. Transposase introduces cleavage bias due to its binding preference associated with GC content of the sequence. Accordingly, GC bias exists due to excess GC-rich peaks in some cell-types causing GC-rich motifs to be excessively strongly differential. Such GC-content bias accounts for variability in the observed coverage for sequencing experiments leading to false-positive peak calls.

In accordance with various embodiments herein, GC content for each TF motif can be adjusted when calling peaks. In some embodiments within the disclosure, to accurately identify the TF motifs, the GC % distribution of peaks can be calculated and then the peaks binned into equal quantile ranges in the GC content distribution. Various methods, in accordance with various embodiments herein, can be used to scan each peak for matches to motif position-weight-matrices (PWMs) for transcription factors from open-access databases of curated, non-redundant transcription factor (TF) binding profiles built directly into the reference data. In accordance with various embodiments herein, a p-value threshold of 1.0×10⁻⁷ can be set, and the background nucleotide frequencies can be set as the observed nucleotide frequencies within the peak regions in each GC bucket. The list of motif matches to detected set of peaks (i.e., motif-peak matches) can be unified across these buckets, thus avoiding GC bias in scanning for motif-peak matches.

The reference data for the various embodiments herein consists of the reference genome sequence and its associated genome annotation, which includes gene and transcript coordinates. Detail regarding the reference data is provided in the “alignment” section above. In one embodiment within the disclosure, the MOODS Python library packaged inside the Cell Ranger™ ATAC Count analysis pipeline, can be used to scan each peak. In one embodiment within the disclosure, the transcription factors database is the JASPAR database that can be built directly into the reference package. JASPAR is an open-access database of curated, non-redundant transcription factor (TF) binding profiles stored as position frequency matrices (PFMs) and TF flexible models (TFFMs) for TFs across multiple species in six taxonomic groups.

Below is a summary of how a peak is annotated to genes and the annotation parameters and procedures used, in accordance with various embodiments herein. It is understood that additional parameters for annotating a peak to a gene can be envisioned in accordance with various embodiments herein.

Annotation of Peaks to Genes

Peaks can be mapped to a gene based on the genomic location of the nearby gene. The general principle of peak annotation in accordance with various embodiments herein are described below. It is understood that other principles for peak annotation in addition to those disclosed herein can be utilized in accordance with various embodiments herein.
The goal of peak annotation is to map peaks to gene symbols, which is the union of all transcripts of a given gene.
A peak can be mapped to multiple genes.
A peak can only be one type of peaks for a given gene, which means a peak cannot be annotated as both a promoter peak and a distal peak of the same gene.
Only protein coding genes are included for annotation.

Peak Annotation Procedure

The peak annotation procedure in accordance with various embodiments herein is described below. It is understood that other parameters and procedures for peak annotation in addition to those disclosed herein can be utilized in accordance with various embodiments herein.
If a peak overlaps with a promoter region (−1000 bp, +100 bp) of any TSS (transcription start site), it can be annotated as a promoter peak of the gene. In other embodiments, if a peak overlaps with a promoter region (−1000 bp, +I000 bp) of any TSS (transcription start site), it can be annotated as a promoter peak of the gene.
If a peak is within 200 kb of the closest TSS, and if it is not a promoter peak of the gene of the closest TSS, it can be annotated as a distal peak of that gene.
If a peak overlaps the body of a transcript, and it is not a promoter nor a distal peak of the gene, it can be annotated as a distal peak of that gene with distance set as zero.
If a peak has not been mapped to any gene at the step, it can be annotated as an intergenic peak without a gene symbol assigned.

TF Motif Enrichment Analysis

The workflow 300 can comprise, at step 390, a transcription factor (TF) motif enrichment analysis. TF motif enrichment analysis includes generating a TF-barcode matrix consisting of the peak-barcode matrix (i.e., pooled cut-site counts for peaks) having a TF motif match, for each motif and each barcode. It is contemplated that the TF motif enrichment can then be utilized for subsequent analysis steps, such as differential accessibility analysis, within various embodiments of the disclosure. Detail related to TF motif enrichment analysis is provided below.

Transcription factors (TFs) play an important role in gene regulation through open, accessible chromatin. As TFs tend to bind at sites containing their cognate motifs, grouping of accessibility measurements at peaks with common motifs is understood to produce a useful enrichment analysis of TFs across single cells. In accordance with various embodiments herein, an integer count for each TF for each cell barcode (i.e., a TF-barcode matrix) can be constructed in the following manner. First, all peaks matched to a given TF are considered, as discovered in the TF motif detection. Second, for every barcode, the cut-site counts across these matched peaks are pooled together in the filtered peak-barcode matrix. This step calculates the total cut-sites in a cell barcode for peaks that share the TF motif. Third, the proportion of cut-sites for a TF within a barcode are calculated out of the total cut-sites for that barcode, which normalizes it to the depth. The enrichment for a TF is then detected by z-scoring the distribution over barcodes of these proportion values for given TF. In some embodiments within the disclosure, to make the analysis robust to outliers, the modified z-score calculated using the median and the scaled median absolute deviation from the median (MAD) is used instead of the mean and standard deviation. These z-scored values are visible when a dataset is loaded, for example into a visualization platform or browser within the various embodiments herein, e.g., the Loupe Cell Browser. Such visualization platform or browser can be built into the differential accessibility analysis of the various embodiments herein. In some embodiments within the disclosure, TF-barcode matrix may not be generated for multi-species experiments or if the motifs files, e.g., motifs.pfm file, is missing from the reference package (for example in custom references).

Differential Accessibility Analysis

The workflow 300 can comprise, at step 392, a differential accessibility analysis that performs differential analysis of TF binding motifs and peaks for identifying differential gene expression between different cells or groups of cells. Various algorithms and statistical models within the disclosure, such as a Negative Binomial (NB2) generalized linear model (GLM), can be employed for the differential accessibility analysis. Detail related to differential accessibility analysis and models employed for the analysis is provided below.

In order to identify transcription factor motifs whose accessibility is specific to each cluster, various embodiments herein, e.g., the Cell Ranger™ ATAC Count analysis pipeline, can test, for each motif and each cluster, whether the in-cluster mean differs from the out-of-cluster mean. Further, in order to find differentially accessible motifs between groups of cells, various embodiments herein, e.g., the Cell Ranger ATAC, can use a Negative Binomial (NB2) generalized linear model (GLM) to discover the cluster specific means and their standard deviations, and then employ a Wald test for inference. In some embodiments within the disclosure, for each cluster, the algorithm can be run on that cluster as compared to running it on all other cells, yielding a list of TF motifs that are differentially expressed in that cluster relative to the rest of the sample. It is understood that, using a GLM framework in accordance with various embodiments herein, can allow modeling the sequencing depth per cell and GC content in peaks per cell directly as covariates. This allows normalizing naturally as part of the model estimation and inference procedure. In accordance with various embodiments herein, the differential enrichment analysis also can be performed for accessibility in peaks using a Poisson generalized linear model (PGLM). In this case, the per cell depth is modeled as the only covariate. In accordance with various embodiments herein, the differential enrichment analysis also can be performed using methods including, but not limited to, standard Gaussian GLM, scABC (which employs a Bayesian passion GLM), and general differential expression (e.g., DESeq, DESEq2, and sSeq etc., which use versions of NB2 GLM under the hood estimates).

By way of example, Equations 1 and 2 below express mathematical relationships between generalized linear model (GLM) and Negative Binomial (NB2) models for discovering the cluster specific means and their standard deviations for finding differentially accessible motifs between groups of cells.

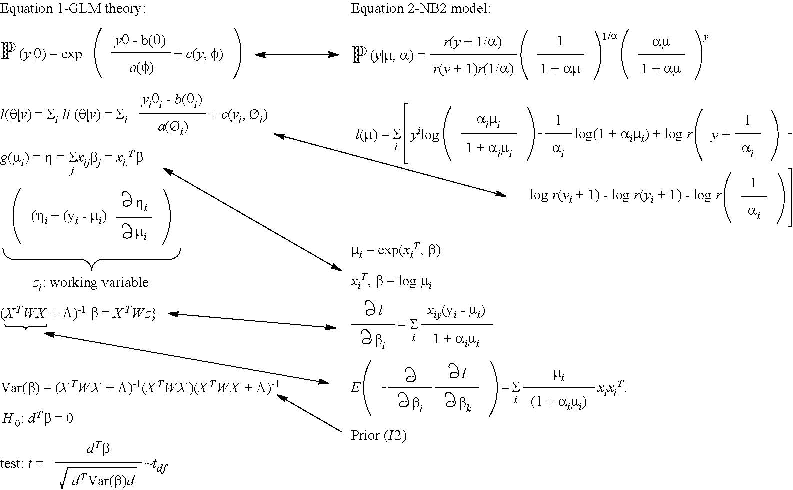

By way of example, the equation below expresses Negative Binomial (NB2) model for basic differential expression analysis of the embodiments herein. In the equation below, Y is the peak-barcode matrix (or such similar matrix) representing an enrichment of each of N cells in M features. Negative binomial model for a single scalar S may be mathematically parametrized as S−NB(scalar mean M, scalar dispersion Alpha), where a scalar is a single variable in the mathematical sense. One could overload the representation for a matrix: Y−NB(M, Alpha), which is really just Y_{ij}˜NB(M_{ij}, Alpha_{ij}) for the i,j-th element. The model below represents that in various embodiments, the matrix M may be factorized as a product of two matrices: X and Beta of dimensions N×P and P×M. Here, P is the number of covariates modeled, such as cluster membership, cell depth, etc., and can be encoded into the known design matrix “X”. Beta is the unknown loading weights for the influence of each covariate on the mean for the count. Beta can be estimated through a fitting procedure known as iteratively reweighted least squares (IRLS). Once Beta is estimated (with fixed Alpha), Alpha can be updated while keeping Beta fixed. Alternatively, Alpha may be permanently fixed via an Empirical Bayes estimate, and the various ways to do that is what distinguishes the various differential enrichment approaches such as DESeq2, sSeq etc. from each other.

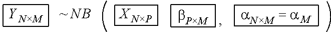

Fit: IRLS for β, can alternative fit a or provide an empirical (e.g., Bayes) estimate
per iteration×each feature: O(NMP²)
convergence: quadratic
Init: Linear Model for log(Y+1)
Reg: Gaussian Priors on R lead to 12 regularization of LL
Design matrix X is an indicator of what cluster each cell belongs to +cell covariates

Visualization

As disclosed herein, the differential accessibility analysis of the various embodiments herein identifies differences in accessibility of peaks and TF binding motifs associated with each identified cell cluster relative to all other identified cell clusters. An output of the differential accessibility analysis capturing differential accessibility of gene regulatory function and specific TF binding motifs associated with each refined cell cluster can be visualized with various embodiments of the disclosure. For example, z-scored values of TF binding motifs can be visualized when a dataset including such values are loaded, for example into a visualization platform or browser within the various embodiments herein, e.g., the Loupe Cell Browser. Such visualization platforms or browsers can be built into the differential accessibility analysis of the various embodiments herein.

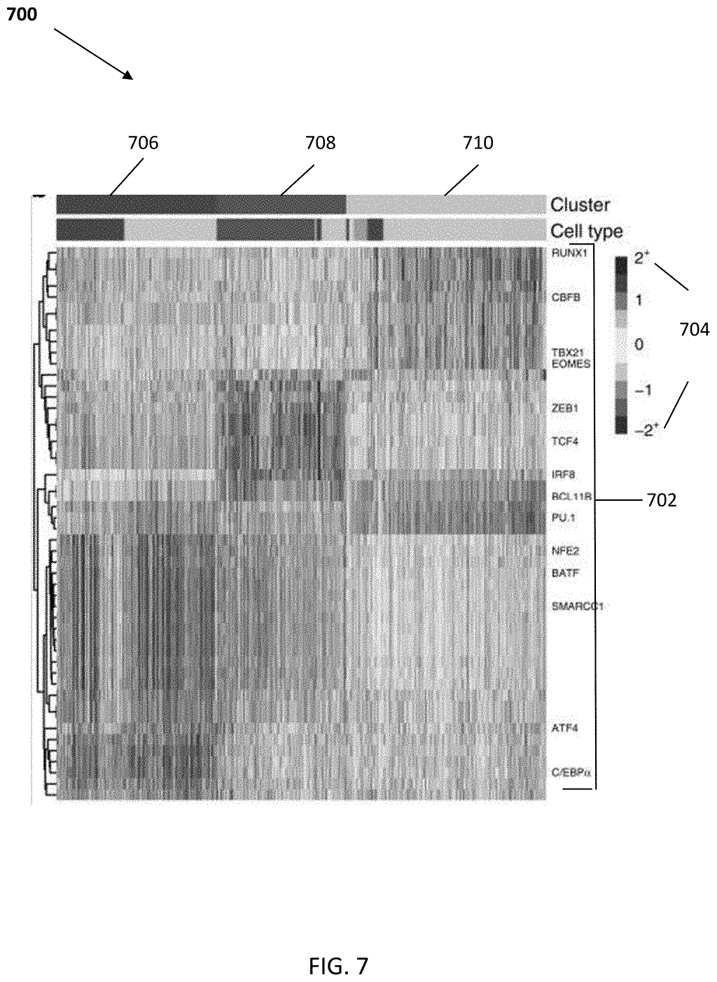

FIG. 7 (Mezger E. et al, Nat Commun. 2018 Sep. 7; 9(1):3647) is an illustration 700 of exemplary heatmap of Z-scores of TF binding motifs in single cell ATAC sequencing cell clusters identified in accordance with various differential accessibility analysis methods. A hierarchical heatmap of accessibility deviation z-scores of TF binding motifs across 2333 single cells (columns) of the 50 most variable TF binding motifs 702 (rows) is represented in illustration 700. Different shades in the heatmap represent difference in accessibility deviation z-scores 704 of TF binding motifs for each cell. As shown in illustration 700, cell subpopulations (upper left panel) co-cluster precisely with the isolated cell types, showing highly concordant cell type-specific accessibility patterns and clustering across clusters 706, 708, and 710 and highly variable TF binding motif accessibility patterns.

As a reminder, it should be appreciated that the methodologies described in the workflow 300 of FIG. 3 and accompanying disclosure can be implemented independently of the methodologies for generating single cell ATAC sequencing data described in FIG. 2. Accordingly, FIG. 3 can be implemented independently of the sequencing data generating workflow as long as it is capable of sufficiently analyzing single cell ATAC sequencing data for identifying genome-wide differential accessibility of gene regulatory elements in accordance with various embodiments.

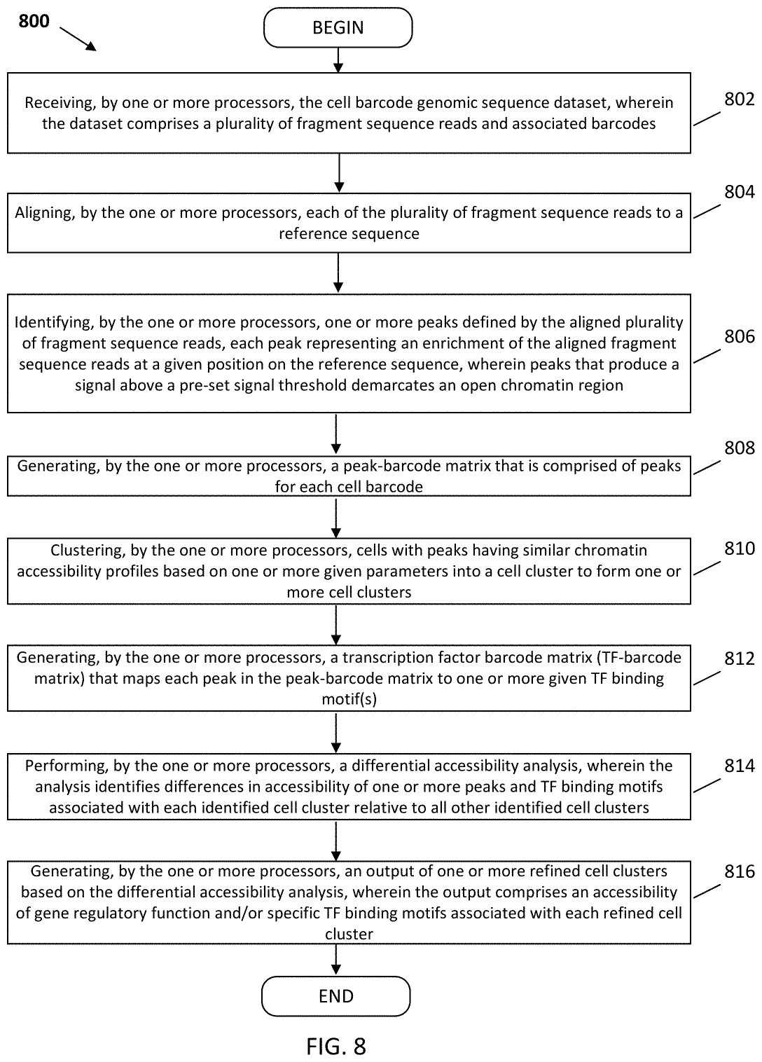

Referring now to FIG. 8, a flowchart is provide illustrating a non-limiting example method 800 for ascertaining differential accessibility of transcription factor (TF) binding motifs in open chromatin regions of cells using a cell barcode genomic sequence dataset, in accordance with various embodiments, in accordance with various embodiments. The method can comprise, at step 802, receiving the cell barcode genomic sequence dataset by one or more processors. In various embodiments, the dataset can include a plurality of fragment sequence reads and associated barcodes.

The method can comprise, at step 804, aligning each of the plurality of fragment sequence reads to a reference sequence by one or more processors. In various embodiments, aligning the fragment sequence reads to the reference sequence can include trimming adapter and/or primer oligonucleotide sequences from one or both ends of the fragment sequence reads. In various embodiments, the reference sequence can include one or more reference genome sequences. In various embodiments, the reference genome sequences can include associated genome annotation, including gene and transcript coordinates. In various embodiments, the reference genome sequences can include single species or multi-species reference genome sequences.

The method can comprise, at step 806, identifying one or more peaks defined by the aligned fragment sequence reads by one or more processors, where each peak can represent an enrichment of the aligned fragment sequence reads at a given position on the reference sequence. In various embodiments, the peaks that produce a signal above a pre-set signal threshold can demarcate an open chromatin region by one or more processors. In various embodiments, the aligned fragment sequence reads can be generated by transposase enzyme cuts during one or more transposition events in one or more accessible open chromatin regions that are mapped to an identified peak along the reference sequence. In various embodiments, identified peaks within a given base-pair (bp) length of each other are merged. In various embodiments, identified peaks within at least 500 bp of each other can be merged. In various embodiments, identified peaks within at least 50 bp, at least 100 bp, at least 200 bp, at least 300 bp, at least 400 bp, at least 500 bp, at least 600 bp, at least 700 bp, at least 800 bp, at least 900 bp, at least 1000 bp, at least 1200 bp, or at least 1500 bp, of each other can be merged. In various embodiments, the peaks identification step can include correcting for GC content bias in the identified peaks. In various embodiments, the method can include associating genes and identifying TF binding motif matches for each identified peak.

The method can comprise, at step 808, generating a peak-barcode matrix by one or more processors, where the peak-barcode matrix represents peaks for each cell barcode. In various embodiments, generating the peak-barcode matrix can include generating a raw peak-barcode matrix representing the peaks for each barcode for all barcodes, and then generating a filtered peak-barcode matrix by filtering out non-cell barcodes from the raw peak-barcode matrix.

In various embodiments, the method can include reducing dimensionality on the peak-barcode matrix. In various embodiments, reducing dimensionality can include a selection of a dimensionality reduction technique. In various embodiments, the dimensionality reduction technique can include, but is not limited to, Principal Component Analysis (PCA), Latent Semantic Analysis (LSA), Probabilistic Latent Semantic Analysis (PLSA), and combinations thereof. In various embodiments, the method may also include visualizing the dataset via t-SNE projection.

The method can comprise, at step 810, clustering cells by one or more processors to group peaks having similar chromatin accessibility profiles, based on one or more given parameters, into a cell cluster to form one or more cell clusters. In various embodiments, the clustering parameter is the closeness of the distance metric calculated using the cut sites in the peaks of the cells. In various embodiments, the clustering parameter is the closeness of the principal components derived from the cut sites in the peaks of the cells. In various embodiments, clustering the cells can include applying a graph-clustering technique including one of K-means clustering, Spherical k-means clustering, and k-medoids clustering.

The method can comprise, at step 812, generating a transcription factor barcode matrix (TF-barcode matrix) by one or more processors. In various embodiments, the TF-barcode matrix includes mapping each peak in the peak-barcode matrix to one or more given TF binding motif(s).

The method can comprise, at step 814, performing a differential accessibility analysis by one or more processors. In various embodiments, the differential accessibility analysis can identify differences in accessibility of one or more identified peaks and TF binding motifs that are associated with each identified cell cluster relative to all other identified cell clusters. In various embodiments, the differential accessibility analysis can utilize a Negative Binomial (NB2) generalized linear model (GLM).

The method can comprise, at step 816, generating an output of one or more refined cell clusters based on the differential accessibility analysis by one or more processors. In various embodiments, such output can include a differential accessibility of gene regulatory function and/or specific TF binding motifs associated with each refined cell cluster.

In various embodiments, the method can include visualizing the dataset and/or one or more outputs generated by the various embodiments herein. In various embodiments, outputs the various embodiments herein, including differential accessibility of gene regulatory function and/or specific TF binding motifs associated with each refined cell cluster, can be visualized.

In various embodiments, the method can include correcting sequencing errors in barcodes in the fragment sequence reads by one or more processors. In various embodiments, the method can include removing the duplicate fragment sequence reads, where such removed duplicate fragment sequence reads include reads arising as a consequence of sequencing and/or PCR amplification. In various embodiments, the method can include selecting fragment sequence reads that maps with a pre-set mapping quality (MAPQ) score, are not mitochondrial sequences, and/or are not chimerically mapped. In various embodiments, the pre-set MAPQ score be a MAPQ score of 30 or more.

In various embodiments, a non-transitory computer-readable medium is provided in which a program is stored for causing a computer to perform a method for ascertaining differential accessibility of transcription factor (TF) binding motifs in open chromatin regions of cells using a cell barcode genomic sequence dataset. The steps within this method can be similar to that provided above, or can vary as needed.

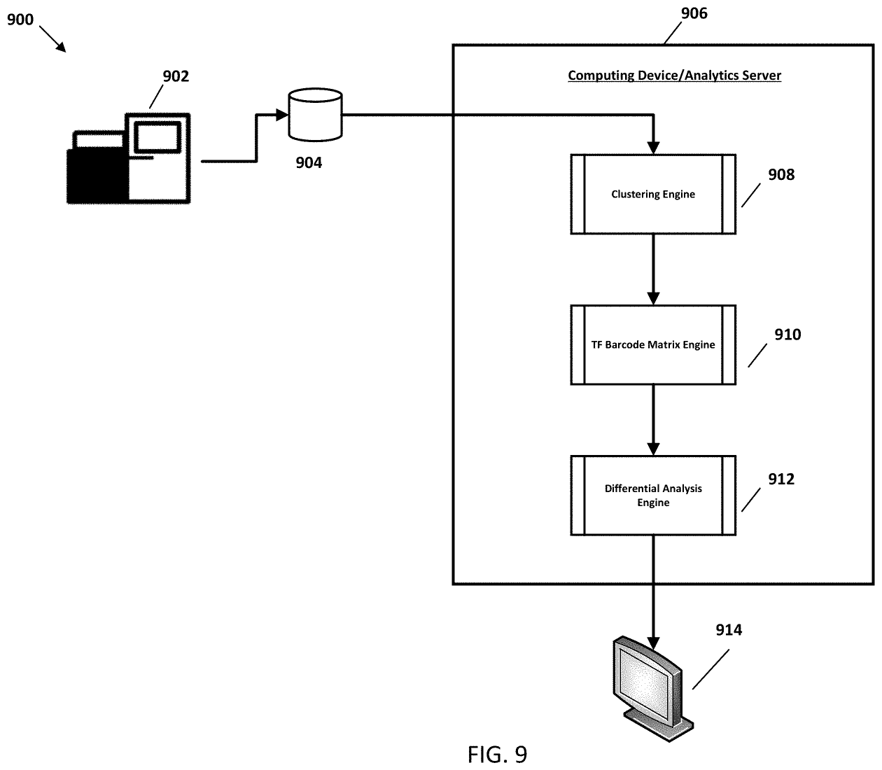

In accordance with various embodiments, FIG. 9 illustrates a non-limiting example system 900 ascertaining differential accessibility of transcription factor (TF) binding motifs in open chromatin regions of cells using a cell barcode genomic sequence dataset, in accordance with various embodiments. The system 900 includes a genomic sequence analyzer 902, a data storage unit 904, a computing device/analytics server 906, and a display 914.

The genomic sequence analyzer 902 can be communicatively connected to the data storage unit 904 by way of a serial bus (if both form an integrated instrument platform) or by way of a network connection (if both are distributed/separate devices). The genomic sequence analyzer 902 can be configured to process and analyze one or more genomic sequence datasets, such as the cell barcode genomic sequence datasets of the various embodiments herein, which includes a plurality of fragment sequence reads and the associated barcodes. In various embodiments, the genomic sequence analyzer 902 can process and analyze one or more genomic sequence datasets that are generated by next-generation sequencing platforms and sequencers such as Illumina® sequencer, MiSeq™, NextSeq™ 500/550 (High Output), HiSeq 2500™ (Rapid Run), HiSeq™ 3000/4000, and NovaSeq.

In various embodiments, the processed and analyzed genomic sequence datasets can then be stored in the data storage unit 904 for subsequent processing. In various embodiments, one or more raw genomic sequence datasets can also be stored in the data storage unit 904 prior to processing and analyzing. Accordingly, in various embodiments, the data storage unit 904 is configured to store one or more genomic sequence datasets, e.g., the cell barcode genomic sequence datasets of the various embodiments herein that includes a plurality of fragment sequence reads and associated barcodes. In various embodiments, the processed and analyzed genomic sequence datasets can be fed to the computing device/analytics server 906 in real-time for further downstream analysis.

In various embodiments, the data storage unit 904 is communicatively connected to the computing device/analytics server 906. In various embodiments, the data storage unit 904 and the computing device/analytics server 906 can be part of an integrated apparatus. In various embodiments, the data storage unit 904 can be hosted by a different device than the computing device/analytics server 906. In various embodiments, the data storage unit 904 and the computing device/analytics server 906 can be part of a distributed network system. In various embodiments, the computing device/analytics server 906 can be communicatively connected to the data storage unit 904 via a network connection that can be either a “hardwired” physical network connection (e.g., Internet, LAN, WAN, VPN, etc.) or a wireless network connection (e.g., Wi-Fi, WLAN, etc.). In various embodiments, the computing device/analytics server 906 can be a workstation, mainframe computer, distributed computing node (part of a “cloud computing” or distributed networking system), personal computer, mobile device, etc.

In various embodiments, the computing device/analytics sever 906 is configured to host a Clustering Engine 908, a Transcription Factor (TF) Barcode Matrix Engine 910, and a Differential Analysis Engine 912.

In various embodiments, the Clustering Engine 908 can be configured to receive one or more genomic sequence datasets, e.g., the cell barcode genomic sequence datasets of the various embodiments herein that includes a plurality of fragment sequence reads and associated barcodes, that are stored in the data storage unit 904. In various embodiments, the Clustering Engine 908 can be configured to receive processed and analyzed genomic sequence datasets from the genomic sequence analyzer 902 in real-time.

In various embodiments, the Clustering Engine 908 can be configured to align each of the plurality of fragment sequence reads to a reference sequence. In various embodiments, aligning the fragment sequence reads to the reference sequence can include trimming adapter and/or primer oligonucleotide sequences from one or both ends of the fragment sequence reads. In various embodiments, the reference sequence can include one or more reference genome sequences. In various embodiments, the reference genome sequences can include associated genome annotation, including gene and transcript coordinates. In various embodiments, the reference genome sequences can include single species or multi-species reference genome sequences.

In various embodiments, the Clustering Engine 908 can be configured to identify one or more peaks that are defined by the aligned plurality of fragment sequence reads, each peak representing an enrichment of the aligned fragment sequence reads at a given position on the reference sequence. In various embodiments, the Clustering Engine 908 is configured to identify peaks that produce a signal above a pre-set signal threshold demarcates an open chromatin region. In various embodiments, the aligned fragment sequence reads can be generated by transposase enzyme cuts during one or more transposition events in one or more accessible open chromatin regions that are mapped to an identified peak along the reference sequence. In various embodiments, the identified peaks within a given base-pair (bp) length of each other are merged. In various embodiments, such bp length for merging peaks can be within a range of 100 bp to 1000 bp. In one embodiment, the bp length can be 500 bp. Further, in various embodiments, the Clustering Engine 908 can be configured to correct GC content bias in the identified peaks. Further, in various embodiments, the Clustering Engine 908 can be configured to associate genes and identifying TF binding motif matches for each identified peak.

In various embodiments, the Clustering Engine 908 can be configured to generate a peak-barcode matrix that is comprised of peaks for each cell barcode. In various embodiments, generating the peak-barcode matrix can include generating a raw peak-barcode matrix representing the peaks for each barcode for all barcodes, and then generating a filtered peak-barcode matrix by filtering out non-cell barcodes from the raw peak-barcode matrix.

Further, in various embodiments, the Clustering Engine 908 can be configured to reduce dimensionality on the peak-barcode matrix by one or more processors. In various embodiments, reducing dimensionality can include a selection of a dimensionality reduction technique. In various embodiments, the dimensionality reduction technique includes, but is not limited to, Principal Component Analysis (PCA), Latent Semantic Analysis (LSA), Probabilistic Latent Semantic Analysis (PLSA), and combinations thereof. In various embodiments, the Clustering Engine 908 can be configured to aid in visualization of the dataset via t-SNE projection.

Lastly, in various embodiments, the Clustering Engine 908 can be configured to cluster cells with peaks that have similar chromatin accessibility profiles, based on one or more given parameters, into a cell cluster to form one or more cell clusters. In various embodiments, the clustering parameter is the closeness of the distance metric calculated using the cut sites in the peaks of the cells. In various embodiments, the clustering parameter is the closeness of the principal components derived from the cut sites in the peaks of the cells. In various embodiments, clustering the cells can include applying a graph-clustering technique including one of K-means clustering, Spherical k-means clustering, and k-medoids clustering.

In various embodiments, any data and analysis generated from the Clustering Engine 908 can be stored in the data store 904 and then subsequently sent/retrieved for utilization by other Engines of the various embodiments herein.

In various embodiments, the TF Barcode Matrix Engine 910 can be configured to generate a transcription factor barcode matrix (TF-barcode matrix) that maps each peak in the peak-barcode matrix to one or more given TF binding motif(s).

In various embodiments, the Differential Analysis Engine 912 can be configured to perform a differential accessibility analysis to identify differences in accessibility of one or more peaks and TF binding motifs that are associated with each identified cell cluster relative to all other identified cell clusters. In various embodiments, the differential accessibility analysis can utilize a Negative Binomial (NB2) generalized linear model (GLM).

In various embodiments, the Differential Analysis Engine 912 can be configured to generate an output of one or more refined cell clusters based on the differential accessibility analysis, where such an output can include a differential accessibility of gene regulatory function and/or specific TF binding motifs associated with each refined cell cluster.

In various embodiments, the computing device/analytics sever 906 can be configured to host other Engines that can be configured to aid in visualizing the dataset and/or one or more outputs generated by the various embodiments herein. In various embodiments, the Engines of the embodiments herein can be configured to aid in visualizing of various outputs including differential accessibility of gene regulatory function and/or specific TF binding motifs associated with each refined cell cluster.

In various embodiments, the computing device/analytics sever 906 can be configured to host other Engines that can be configured to correct sequencing errors in barcodes in the fragment sequence reads by one or more processors. Further, in various embodiments, the computing device/analytics sever 906 can be configured to host other Engines that can be configured to remove duplicate fragment sequence reads, where the removed duplicate fragment sequence reads include reads arising as a consequence of sequencing and/or PCR amplification. Lastly, in various embodiments, the computing device/analytics sever 906 can be configured to select fragment sequence reads that maps with a pre-set mapping quality (MAPQ) score, are not mitochondrial sequences, and/or are not chimerically mapped. In various embodiments, the pre-set MAPQ score be a MAPQ score of 30 or more.

After the differential accessibility analysis has been performed and an output of one or more refined cell clusters based on the differential accessibility analysis has been generated, it can be displayed as a result or summary on a display or client terminal 914 that is communicatively connected to the computing device/analytics server 906. In various embodiments, the display or client terminal 914 can be a thin client computing device. In various embodiments, the display or client terminal 914 can be a personal computing device having a web browser (e.g., INTERNET EXPLORER™, FIREFOX™, SAFARI™, etc.) that can be used to control the operation of the genomic sequence analyzer 902, data store 904, Clustering Engine 908, TF Barcode Matrix Engine 910, and the Differential Analysis Engine 912.

### Experimental Results

### GC Bias Exists Due to Excess GC-Rich Peaks

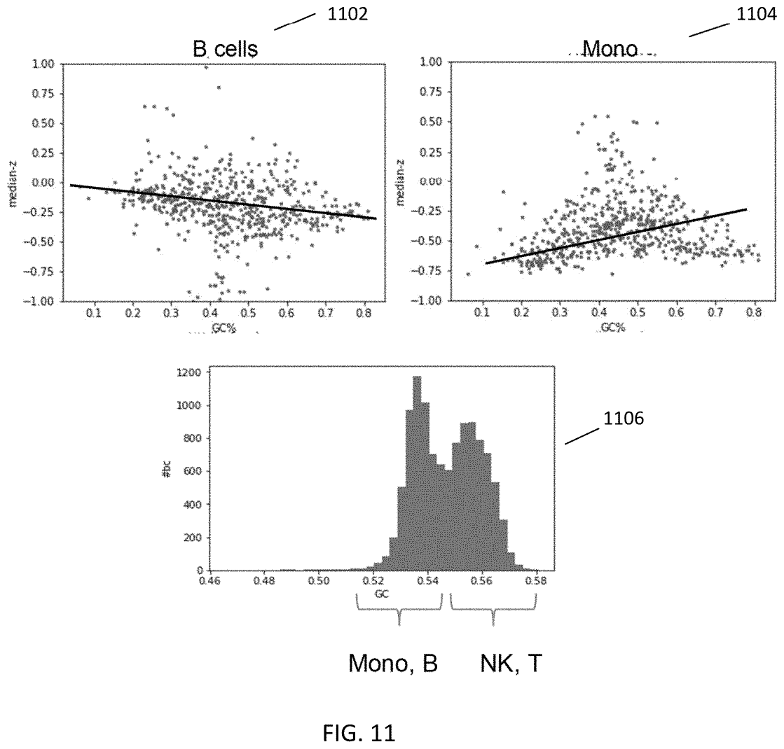

FIG. 11 is a collection of plots 1102, 1104, and 1106 demonstrating that GC bias exists due to excess GC-rich peaks in some cell types causing GC-rich transcription factor (TF) motifs to be excessively, strongly differential. It is known that TFs bind to specific motifs on the open, accessible parts of the chromatin. Cell types differ from each other in the set of peaks based on the accessibility of open chromatin regions. Some cell types have higher GC content in these set of peaks. As a result, the TFs that bind to GC-rich motifs show a high accessibility in their binding regions in the cell types that have higher GC content in these set of peaks when compared to other cell types. Accordingly, such cells appear to be strongly differentially accessible—excessively so—because of the GC bias. Plots 1102 and 1104 show the “z score” normalized accessibility signal for each TF within the indicated cell type (i.e., B cells and monocyte (Mono)) associated peaks, plotted against the GC content of the known binding motif sequence for each TF. As seen here, plots 1102 and 1104 demonstrate a correlation between the accessibility signal for each TF within each indicated cell type-associated peaks and the GC content of the known binding motif sequence for each TF, as captured by the black line. Plot 1106 illustrates the distribution of GC content in peaks for each barcode and shows two humps corresponding to the indicated cell types. The plots confirm that GC bias exists due to excess GC-rich peaks in some cell types causing GC-rich TF motifs to be excessively strongly differential.

### GC Content and Peak Size Influence Chromatin Accessibility Analysis

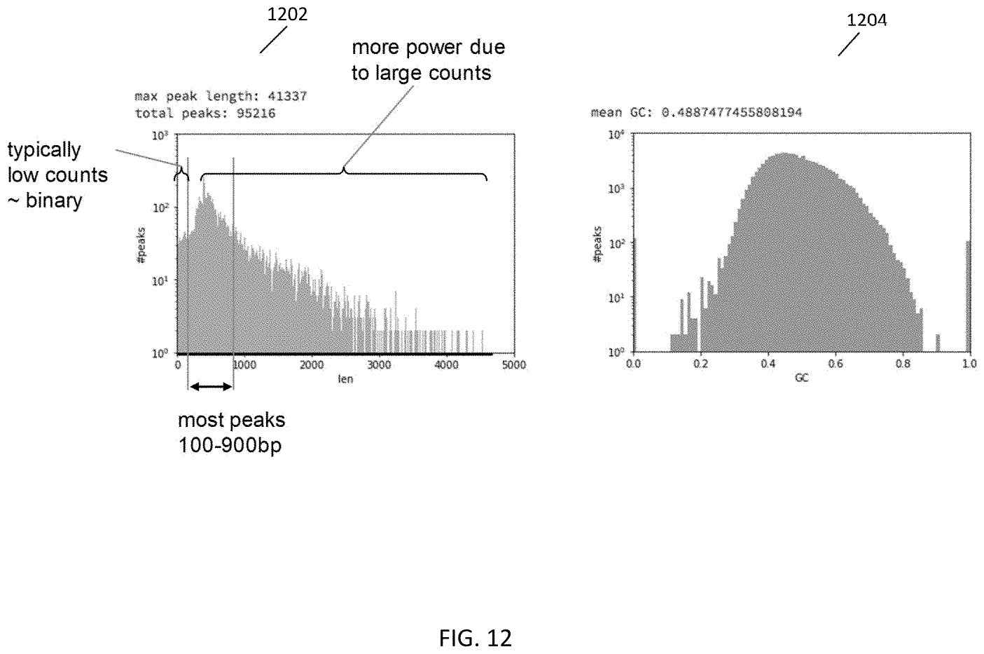

FIG. 12 are plots 1202 and 1204 demonstrating that GC content and peak size both influence chromatin accessibility analysis. Plot 1202 illustrates a distribution of counts (i.e., accessibility of open chromatin region) observed in a peak plotted against peak size. Plot 1204 illustrates a distribution of counts (i.e., accessibility of open chromatin region) plotted against GC % of the peak. As seen here, plots 1202 and 1204 demonstrates that both GC content and the peak size influence the analysis of chromatin accessibility as seen from the distributions. Accordingly, both GC content and peak size are factored in the chromatin accessibility analysis in accordance various embodiments herein.

### Fisher's Exact Test for Outlier Peaks

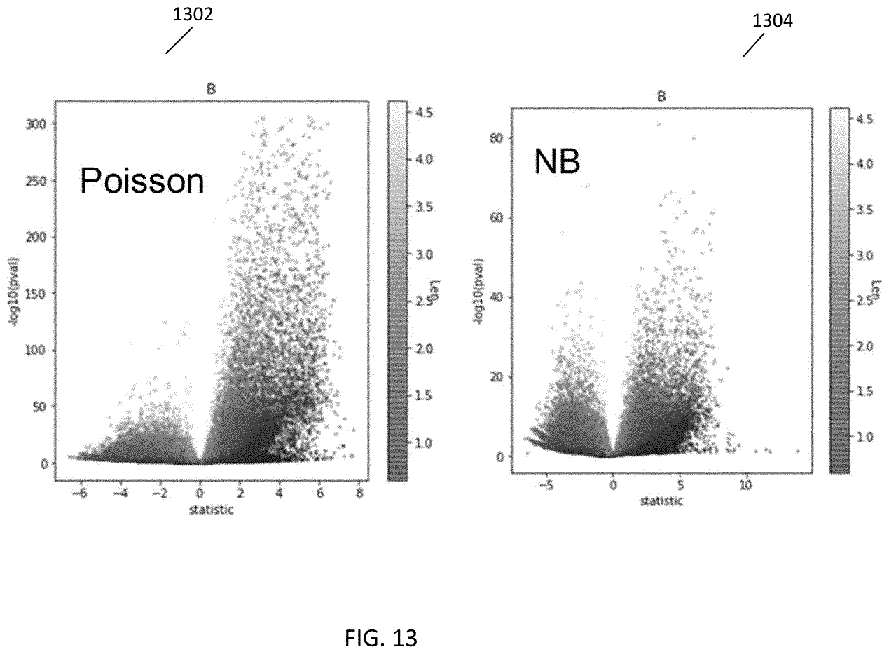

FIG. 13 are plots 1302 and 1304 demonstrating non-limiting examples of testing for outlier peaks via Fisher's exact test, in accordance with various embodiments. Plots 1302 and 1304 with −1*log(pval) plotted against statistic mean: for each feature (e.g., peak or TF motif), the log of ratio of mean accessibility in cluster 1 vs cluster 2 is the statistic on the x axis. The y axis is the probability of that feature (e.g., peak or TF motif) being differentially accessible in cluster 1 vs 2. It is understood that the extreme pvalues (i.e., more confident differentially accessible features) usually correspond to large statistic values. Low pvalues at extreme values of the statistic can be an issue. In various embodiments, such issues with outlier points shown in plot 1302, which utilizes a Poisson model, can be tested and fixed via Fisher's exact test as shown in plot 1304. Accordingly, issues with outlier points described herein are tested and fixed, for example, via Fisher's exact test, in accordance various embodiments herein.

### Computer System

In various embodiments, the methods for ascertaining differential accessibility of transcription factor (TF) binding motifs in open chromatin regions of cells using a cell barcode genomic sequence dataset can be implemented via computer software or hardware. That is, as depicted in FIG. 9, the methods disclosed herein can be implemented on a computing device/analytics sever 906 that includes a Clustering Engine 908 and, optionally, a Transcription Factor (TF) Barcode Matrix Engine 910 and a Differential Analysis_Engine 912. In various embodiments, the computing device/analytics sever 906 can be communicatively connected to a genomic sequence analyzer 902, a data store 904, and a display or client terminal 914, via a direct connection or through an internet connection.

It should be appreciated that the various engines depicted in FIG. 9 can be combined or collapsed into a single engine, component or module, depending on the requirements of the particular application or system architecture. Moreover, in various embodiments, the Clustering Engine 908, and, optionally, the Transcription Factor (TF) Barcode Matrix Engine 910 and Differential Analysis Engine 912, can comprise additional engines or components as needed by the particular application or system architecture.

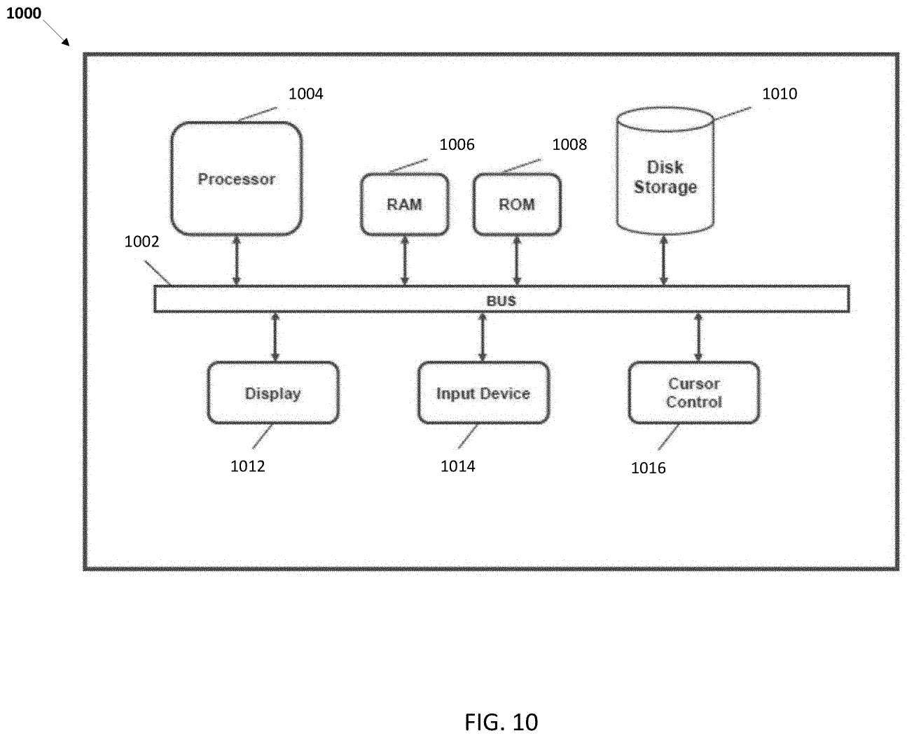

FIG. 10 is a block diagram that illustrates a computer system 1000, upon which embodiments of the present teachings may be implemented. In various embodiments of the present teachings, computer system 1000 can include a bus 1002 or other communication mechanism for communicating information, and a processor 1004 coupled with bus 1002 for processing information. In various embodiments, computer system 1000 can also include a memory, which can be a random access memory (RAM) 1006 or other dynamic storage device, coupled to bus 1002 for determining instructions to be executed by processor 1004. Memory also can be used for storing temporary variables or other intermediate information during execution of instructions to be executed by processor 1004. In various embodiments, computer system 1000 can further include a read only memory (ROM) 1008 or other static storage device coupled to bus 1002 for storing static information and instructions for processor 1004. A storage device 1010, such as a magnetic disk or optical disk, can be provided and coupled to bus 1002 for storing information and instructions.

In various embodiments, computer system 1000 can be coupled via bus 1002 to a display 1012, such as a cathode ray tube (CRT) or liquid crystal display (LCD), for displaying information to a computer user. An input device 1014, including alphanumeric and other keys, can be coupled to bus 1002 for communicating information and command selections to processor 1004. Another type of user input device is a cursor control 1016, such as a mouse, a trackball or cursor direction keys for communicating direction information and command selections to processor 1004 and for controlling cursor movement on display 1012. This input device 1014 typically has two degrees of freedom in two axes, a first axis (i.e., x) and a second axis (i.e., y), that allows the device to specify positions in a plane. However, it should be understood that input devices 1014 allowing for 3 dimensional (x, y and z) cursor movement are also contemplated herein.

Consistent with certain implementations of the present teachings, results can be provided by computer system 1000 in response to processor 1004 executing one or more sequences of one or more instructions contained in memory 1006. Such instructions can be read into memory 1006 from another computer-readable medium or computer-readable storage medium, such as storage device 1010. Execution of the sequences of instructions contained in memory 1006 can cause processor 1004 to perform the processes described herein. Alternatively hard-wired circuitry can be used in place of or in combination with software instructions to implement the present teachings. Thus implementations of the present teachings are not limited to any specific combination of hardware circuitry and software.

The term “computer-readable medium” (e.g., data store, data storage, etc.) or “computer-readable storage medium” as used herein refers to any media that participates in providing instructions to processor 1004 for execution. Such a medium can take many forms, including but not limited to, non-volatile media, volatile media, and transmission media. Examples of non-volatile media can include, but are not limited to, optical, solid state, magnetic disks, such as storage device 1010. Examples of volatile media can include, but are not limited to, dynamic memory, such as memory 1006. Examples of transmission media can include, but are not limited to, coaxial cables, copper wire, and fiber optics, including the wires that comprise bus 1002.

Common forms of computer-readable media include, for example, a floppy disk, a flexible disk, hard disk, magnetic tape, or any other magnetic medium, a CD-ROM, any other optical medium, punch cards, paper tape, any other physical medium with patterns of holes, a RAM, PROM, and EPROM, a FLASH-EPROM, any other memory chip or cartridge, or any other tangible medium from which a computer can read.

In addition to computer readable medium, instructions or data can be provided as signals on transmission media included in a communications apparatus or system to provide sequences of one or more instructions to processor 1004 of computer system 1000 for execution. For example, a communication apparatus may include a transceiver having signals indicative of instructions and data. The instructions and data are configured to cause one or more processors to implement the functions outlined in the disclosure herein. Representative examples of data communications transmission connections can include, but are not limited to, telephone modem connections, wide area networks (WAN), local area networks (LAN), infrared data connections, NFC connections, etc.

It should be appreciated that the methodologies described herein flowcharts, diagrams and accompanying disclosure can be implemented using computer system 1000 as a standalone device or on a distributed network of shared computer processing resources such as a cloud computing network.

The methodologies described herein may be implemented by various means depending upon the application. For example, these methodologies may be implemented in hardware, firmware, software, or any combination thereof. For a hardware implementation, the processing unit may be implemented within one or more application specific integrated circuits (ASICs), digital signal processors (DSPs), digital signal processing devices (DSPDs), programmable logic devices (PLDs), field programmable gate arrays (FPGAs), processors, controllers, micro-controllers, microprocessors, electronic devices, other electronic units designed to perform the functions described herein, or a combination thereof.

In various embodiments, the methods of the present teachings may be implemented as firmware and/or a software program and applications written in conventional programming languages such as C, C++, Python, etc. If implemented as firmware and/or software, the embodiments described herein can be implemented on a non-transitory computer-readable medium in which a program is stored for causing a computer to perform the methods described above. It should be understood that the various engines described herein can be provided on a computer system, such as computer system 1000, whereby processor 1004 would execute the analyses and determinations provided by these engines, subject to instructions provided by any one of, or a combination of, memory components 1006/1008/1010 and user input provided via input device 1014.

While the present teachings are described in conjunction with various embodiments, it is not intended that the present teachings be limited to such embodiments. On the contrary, the present teachings encompass various alternatives, modifications, and equivalents, as will be appreciated by those of skill in the art.

In describing the various embodiments, the specification may have presented a method and/or process as a particular sequence of steps. However, to the extent that the method or process does not rely on the particular order of steps set forth herein, the method or process should not be limited to the particular sequence of steps described, and one skilled in the art can readily appreciate that the sequences may be varied and still remain within the spirit and scope of the various embodiments.

### Recitation of Embodiments

Embodiment 1. A method for ascertaining differential accessibility of transcription factor (TF) binding motifs in open chromatin regions of cells using a cell barcode genomic sequence dataset, the method comprising: receiving, by one or more processors, the cell barcode genomic sequence dataset, wherein the dataset comprises a plurality of fragment sequence reads and associated barcodes; aligning, by the one or more processors, each of the plurality of fragment sequence reads to a reference sequence; identifying, by the one or more processors, one or more peaks defined by the aligned plurality of fragment sequence reads, each peak representing an enrichment of the aligned fragment sequence reads at a given position on the reference sequence, wherein peaks that produce a signal above a pre-set signal threshold demarcates an open chromatin region; generating, by the one or more processors, a peak-barcode matrix that is comprised of peaks for each cell barcode; clustering, by the one or more processors, cells with peaks having similar chromatin accessibility profiles based on one or more given parameters into a cell cluster to form one or more cell clusters; generating, by the one or more processors, a transcription factor (TF) barcode matrix that maps each peak in the peak-barcode matrix to one or more given TF binding motif(s); performing, by the one or more processors, a differential accessibility analysis, wherein the analysis identifies differences in accessibility of one or more peaks and TF binding motifs associated with each identified cell cluster relative to all other identified cell clusters; and generating, by the one or more processors, an output of one or more refined cell clusters based on the differential accessibility analysis, wherein the output comprises a differential accessibility of gene regulatory function and/or specific TF binding motifs associated with each refined cell cluster.

Embodiment 2. The method of Embodiment 1, further comprising visualizing the dataset and/or one or more outputs.

Embodiment 3. The method of Embodiment 2, the output comprises differential accessibility of gene regulatory function and/or specific TF binding motifs associated with each refined cell cluster.

Embodiment 4. The method of Embodiment 1, wherein the aligned fragment sequence reads are generated by transposase enzyme cuts during one or more transposition events in one or more accessible open chromatin regions mapped to an identified peak along the reference sequence.

Embodiment 5. The method of Embodiment 1, wherein aligning the fragment sequence reads to the reference sequence further comprises trimming adapter and/or primer oligonucleotide sequences from one or both ends of the fragment sequence reads.

Embodiment 6. The method of Embodiment 1, wherein the reference sequence comprises one or more reference genome sequences, wherein the one or more reference genome sequences include associated genome annotation.

Embodiment 7. The method of Embodiment 6, wherein the one or more reference genome sequences include single species or multi-species reference genome sequences.

Embodiment 8. The method of Embodiment 1, wherein the identified peaks within a given base-pair (bp) length of each other are merged.

Embodiment 9. The method of Embodiment 8, wherein the given bp length is within a range of 100 bp to 1000 bp.

Embodiment 10. The method of Embodiment 9, wherein the given bp length is 500 bp.

Embodiment 11. The method of Embodiment 1, wherein the identifying peaks comprises correcting for GC content bias in the identified peaks.

Embodiment 12. The method of Embodiment 1, wherein generating the peak-barcode matrix comprises: (a) generating a raw peak-barcode matrix comprising peaks for each barcode for all barcodes; and (b) generating a filtered peak-barcode matrix by filtering out non-cell barcodes from the raw peak-barcode matrix.

Embodiment 13. The method of Embodiment 1, wherein the clustering parameter is a closeness of a distance metric calculated using cut sites in the peaks of the cells.

Embodiment 14. The method of Embodiment 1, further comprising at least one of the following: correcting sequencing errors in barcodes in the fragment sequence reads; removing duplicate fragment sequence reads, wherein the removed duplicate fragment sequence reads include reads arising as a consequence of sequencing and/or PCR amplification; and selecting fragment sequence reads that maps with a pre-set mapping quality (MAPQ) score, are not mitochondrial sequences, and/or are not chimerically mapped.

Embodiment 15. The method of Embodiment 14, wherein the pre-set MAPQ score comprises a MAPQ score of 30 or more.

Embodiment 16. The method of Embodiment 1, further comprising associating genes and identifying TF binding motif matches for each identified peak.

Embodiment 17. The method of Embodiment 1, further comprising reducing dimensionality on the peak-barcode matrix.

Embodiment 18. The method of Embodiment 17, wherein reducing dimensionality comprises a selection of a dimensionality reduction technique.

Embodiment 19. The method of Embodiment 18, wherein the dimensionality reduction technique is selected from the group consisting of Principal Component Analysis (PCA), Latent Semantic Analysis (LSA), Probabilistic Latent Semantic Analysis (PLSA), and combinations thereof.

Embodiment 20. The method of Embodiment 19, further comprising applying a graph-clustering technique comprising one of K-means clustering, Spherical k-means clustering, and k-medoids clustering.

Embodiment 21. The method of Embodiment 17, further comprising visualizing the dataset via t-SNE projection.

Embodiment 22. The method of Embodiment 1, wherein the differential accessibility analysis uses a Negative Binomial (NB2) generalized linear model (GLM).

Embodiment 23. The method of Embodiment 1, further comprising generating peak calls from the barcode genomic sequence dataset, the generating of peak calls comprising: determining a cumulative signal at each position along a signal track of the genome across a constant, moving window; applying an initial threshold on the signal track; applying a multi-component mixture model to the dataset, wherein an expectation maximum is initiated on the data set and converged to a final distribution; generating a global threshold as a probability of the components of the multi-component mixture model; and applying the global threshold across a full data set for making peak calls.

Embodiment 24. The method of Embodiment 23, further comprising: applying a masking threshold on a portion of the dataset, wherein the threshold is a percentage value, wherein all counts above the threshold are masked and all counts below the threshold are compiled into a data subset; determining a first maximum likelihood fitting of a first discrete probability distribution for the data subset; identifying a first residual signal data set from the first maximum likelihood fitting; determining a second maximum likelihood fitting of a second discrete probability distribution on the first residual data set; generating a truncated data set from at least one of the first and second likelihood fittings; applying a first expectation maximization on the truncated data set to fit the truncated data set to background noise data; applying a second expectation maximization on the full data set to generate a final distribution; and generating the global threshold as a function of the first and second discrete probability distributions.

Embodiment 25. The method of Embodiment 23, further comprising: performing a wavelet transform on a local signal within the data to produce a wavelet transformed signal; identifying a putative peak as a local maxima of the wavelet transformed signal; bounding the putative peak at a maximal prominence percentage of the putative peak; performing a loop through a putative peak region, including calculating a beta distribution across the putative peak region; determining a local threshold as a function of signal to noise ratio of the putative peak region; calling the putative peak as a true peak when a probability mass percentage of the beta distribution is above the local threshold.

Embodiment 26. The method of Embodiment 23, wherein the constant, moving window has a length of about 400 bp.

Embodiment 27. The method of Embodiment 23, wherein the multi-component mixture model is a 3-component mixture model, wherein the components are discrete probability distributions selected from the group consisting of a zero-inflated noise component, a negative binomial noise component, and a combination thereof.

Embodiment 28. The method of Embodiment 23, wherein the global threshold is a fixed tail probability of the components of the multi-component mixture model.

Embodiment 29. The method of Embodiment 24, wherein the masking threshold is between about 5% to about 50%.

Embodiment 30. The method of Embodiment 24, wherein the first discrete probability distribution is a zero-inflated noise component with a single geometric distribution.

Embodiment 31. The method of Embodiment 24, wherein the identifying of the first residual signal data set comprises: generating weighted counts for the subset based on the determined first maximum likelihood fitting, and extracting data exceeding the fit as the first residual signal data set.

Embodiment 32. The method of Embodiment 24, wherein the second discrete probability distribution is a first negative binomial noise component.

Embodiment 33. The method of Embodiment 24, wherein the first expectation maximization includes applying a 2-component mixture model on the truncated data set.

Embodiment 34. The method of Embodiment 24, wherein the second expectation maximization includes applying a 3-component mixture model on the truncated data set.

Embodiment 35. The method of Embodiment 25, further comprising performing a wavelet transform on the local signal, with a fixed wavelet width, within the data to produce a wavelet transformed signal.

Embodiment 36. The method of Embodiment 35, wherein the fixed wavelet width is between about 50 bp to about 2000 bp.

Embodiment 37. The method of Embodiment 36, wherein the fixed wavelet width is about 300 bp.

Embodiment 38. The method of Embodiment 25, wherein the maximal prominence percentage is between about 50% and about 95%.

Embodiment 39. The method of Embodiment 38, wherein the maximal prominence percentage is 95%.

Embodiment 40. The method of Embodiment 25, wherein performing a loop through the putative peak region comprises: calculating a first median real signal inside the putative peak region as the peak signal; calculating a second median real signal at a given width in the putative peak region but outside all other observed putative peaks; and calculating the beta distribution with a prior and a Bayesian update;

Embodiment 41. The method of Embodiment 40, wherein the width is one or more widths, and the width is selected from the group consisting of 1× the peak width, 3× the peak width, 5× the peak width, and combinations thereof.

Embodiment 42. The method of Embodiment 25, wherein the probability mass percentage is at least about 95%.

Embodiment 43. A non-transitory computer-readable medium in which a program is stored for causing a computer to perform a method for ascertaining differential accessibility of transcription factor (TF) binding motifs in open chromatin regions of cells using a cell barcode genomic sequence dataset, the method comprising: receiving, by one or more processors, the cell barcode genomic sequence dataset, wherein the dataset comprises a plurality of fragment sequence reads and associated barcodes; aligning, by the one or more processors, each of the plurality of fragment sequence reads to a reference sequence; identifying, by the one or more processors, one or more peaks defined by the aligned plurality of fragment sequence reads, each peak representing an enrichment of the aligned fragment sequence reads at a given position on the reference sequence, wherein peaks that produce a signal above a pre-set signal threshold demarcates an open chromatin region; generating, by the one or more processors, a peak-barcode matrix that is comprised of peaks for each cell barcode; clustering, by the one or more processors, cells with peaks having similar chromatin accessibility profiles based on one or more given parameters into a cell cluster to form one or more cell clusters; generating, by the one or more processors, a transcription factor (TF) barcode matrix that maps each peak in the peak-barcode matrix to one or more given TF binding motif(s); performing, by the one or more processors, a differential accessibility analysis, wherein the analysis identifies differences in accessibility of one or more peaks and TF binding motifs associated with each identified cell cluster relative to all other identified cell clusters; and generating, by the one or more processors, an output of one or more refined cell clusters based on the differential accessibility analysis, wherein the output comprises a differential accessibility of gene regulatory function and/or specific TF binding motifs associated with each refined cell cluster.

Embodiment 44. The non-transitory computer-readable medium of Embodiment 43, further comprising visualizing the dataset and/or one or more outputs.

Embodiment 45. The non-transitory computer-readable medium of Embodiment 44, the output comprises differential accessibility of gene regulatory function and/or specific TF binding motifs associated with each refined cell cluster.

Embodiment 46. The non-transitory computer-readable medium of Embodiment 43, wherein the aligned fragment sequence reads are generated by transposase enzyme cuts during one or more transposition events in one or more accessible open chromatin regions mapped to an identified peak along the reference sequence.

Embodiment 47. The non-transitory computer-readable medium of Embodiment 43, wherein aligning the fragment sequence reads to the reference sequence further comprises trimming adapter and/or primer oligonucleotide sequences from one or both ends of the fragment sequence reads.

Embodiment 48. The non-transitory computer-readable medium of Embodiment 43, wherein the reference sequence comprises one or more reference genome sequences, wherein the one or more reference genome sequences include associated genome annotation.

Embodiment 49. The non-transitory computer-readable medium of Embodiment 48, wherein the one or more reference genome sequences include single species or multi-species reference genome sequences.

Embodiment 50. The non-transitory computer-readable medium of Embodiment 43, wherein the identified peaks within a given base-pair (bp) length of each other are merged.

Embodiment 51. The non-transitory computer-readable medium of Embodiment 50, wherein the given bp length is within a range of 100 bp to 1000 bp.

Embodiment 52. The non-transitory computer-readable medium of Embodiment 51, wherein the given bp length is 500 bp.

Embodiment 53. The non-transitory computer-readable medium of Embodiment 43, wherein the identifying peaks comprises correcting for GC content bias in the identified peaks.

Embodiment 54. The non-transitory computer-readable medium of Embodiment 43, wherein generating the peak-barcode matrix comprises: (a) generating a raw peak-barcode matrix comprising peaks for each barcode for all barcodes; and (b) generating a filtered peak-barcode matrix by filtering out non-cell barcodes from the raw peak-barcode matrix.

Embodiment 55. The non-transitory computer-readable medium of Embodiment 43, wherein the clustering parameter is a closeness of a distance metric calculated using cut sites in the peaks of the cells.

Embodiment 56. The non-transitory computer-readable medium of Embodiment 43, further comprising at least one of the following: correcting sequencing errors in barcodes in the fragment sequence reads; removing duplicate fragment sequence reads, wherein the removed duplicate fragment sequence reads include reads arising as a consequence of sequencing and/or PCR amplification; and selecting fragment sequence reads that maps with a pre-set mapping quality (MAPQ) score, are not mitochondrial sequences, and/or are not chimerically mapped.

Embodiment 57. The non-transitory computer-readable medium of Embodiment 56, wherein the pre-set MAPQ score comprises a MAPQ score of 30 or more.

Embodiment 58. The non-transitory computer-readable medium of Embodiment 43, further comprising associating genes and identifying TF binding motif matches for each identified peak.

Embodiment 59. The non-transitory computer-readable medium of Embodiment 43, further comprising reducing dimensionality on the peak-barcode matrix.

Embodiment 60. The non-transitory computer-readable medium of Embodiment 59, wherein reducing dimensionality comprises a selection of a dimensionality reduction technique.

Embodiment 61. The non-transitory computer-readable medium of Embodiment 60, wherein the dimensionality reduction technique is selected from the group consisting of Principal Component Analysis (PCA), Latent Semantic Analysis (LSA), Probabilistic Latent Semantic Analysis (PLSA), and combinations thereof.

Embodiment 62. The non-transitory computer-readable medium of Embodiment 61, further comprising applying a graph-clustering technique comprising one of K-means clustering, Spherical k-means clustering, and k-medoids clustering.

Embodiment 63. The non-transitory computer-readable medium of Embodiment 59, further comprising visualizing the dataset via t-SNE projection.

Embodiment 64. The non-transitory computer-readable medium of Embodiment 43, wherein the differential accessibility analysis uses a Negative Binomial (NB2) generalized linear model (GLM).

Embodiment 65. The non-transitory computer-readable medium of Embodiment 43, further comprising generating peak calls from the barcode genomic sequence dataset, the generating of peak calls comprising: determining a cumulative signal at each position along a signal track of the genome across a constant, moving window; applying an initial threshold on the signal track; applying a multi-component mixture model to the dataset, wherein an expectation maximum is initiated on the data set and converged to a final distribution; generating a global threshold as a probability of the components of the multi-component mixture model; and applying the global threshold across the full data set for making peak calls.

Embodiment 66. The non-transitory computer-readable medium of Embodiment 65, further comprising: applying a masking threshold on a portion of the dataset, wherein the threshold is a percentage value, wherein all counts above the threshold are masked and all counts below the threshold are compiled into a data subset; determining a first maximum likelihood fitting of a first discrete probability distribution for the data subset; identifying a first residual signal data set from the first maximum likelihood fitting; determining a second maximum likelihood fitting of a second discrete probability distribution on the first residual data set; generating a truncated data set from at least one of the first and second likelihood fittings; applying a first expectation maximization on the truncated data set to fit the truncated data set to background noise data; applying a second expectation maximization on the full data set to generate a final distribution; and generating the global threshold as a function of the first and second discrete probability distributions.

Embodiment 67. The non-transitory computer-readable medium of Embodiment 65, further comprising: performing a wavelet transform on a local signal within the data to produce a wavelet transformed signal; identifying a putative peak as a local maxima of the wavelet transformed signal; bounding the putative peak at a maximal prominence percentage of the putative peak; performing a loop through a putative peak region, including calculating a beta distribution across the putative peak region; determining a local threshold as a function of signal to noise ratio of the putative peak region; calling the putative peak as a true peak when a probability mass percentage of the beta distribution is above the local threshold.

Embodiment 68. The non-transitory computer-readable medium of Embodiment 65, wherein the constant, moving window has a length of about 400 bp.

Embodiment 69. The non-transitory computer-readable medium of Embodiment 65, wherein the multi-component mixture model is a 3-component mixture model, wherein the components are discrete probability distributions selected from the group consisting of a zero-inflated noise component, a negative binomial noise component, and a combination thereof.

Embodiment 70. The non-transitory computer-readable medium of Embodiment 65, wherein the global threshold is a fixed tail probability of the components of the multi-component mixture model.

Embodiment 71. The non-transitory computer-readable medium of Embodiment 66, wherein the masking threshold is between about 5% to about 50%.

Embodiment 72. The non-transitory computer-readable medium of Embodiment 66, wherein the first discrete probability distribution is a zero-inflated noise component with a single geometric distribution.

Embodiment 73. The non-transitory computer-readable medium of Embodiment 66, wherein the identifying of the first residual signal data set comprises: generating weighted counts for the subset based on the determined first maximum likelihood fitting, and extracting data exceeding the fit as the first residual signal data set.

Embodiment 74. The non-transitory computer-readable medium of Embodiment 66, wherein the second discrete probability distribution is a first negative binomial noise component.

Embodiment 75. The non-transitory computer-readable medium of Embodiment 66, wherein the first expectation maximization includes applying a 2-component mixture model on the truncated data set.

Embodiment 76. The non-transitory computer-readable medium of Embodiment 66, wherein the second expectation maximization includes applying a 3-component mixture model on the truncated data set.

Embodiment 77. The non-transitory computer-readable medium of Embodiment 67, further comprising performing a wavelet transform on the local signal, with a fixed wavelet width, within the data to produce a wavelet transformed signal.

Embodiment 78. The non-transitory computer-readable medium of Embodiment 77, wherein the fixed wavelet width is between about 50 bp to about 2000 bp.

Embodiment 79. The non-transitory computer-readable medium of Embodiment 78, wherein the fixed wavelet width is about 300 bp.

Embodiment 80. The non-transitory computer-readable medium of Embodiment 67, wherein the maximal prominence percentage is between about 50% and about 95%.

Embodiment 81. The non-transitory computer-readable medium of Embodiment 80, wherein the maximal prominence percentage is 95%.

Embodiment 82. The non-transitory computer-readable medium of Embodiment 67, wherein performing a loop through the putative peak region comprises: calculating a first median real signal inside the putative peak region as the peak signal; calculating a second median real signal at a given width in the putative peak region but outside all other observed putative peaks; and calculating the beta distribution with a prior and a Bayesian update;

Embodiment 83. The non-transitory computer-readable medium of Embodiment 82, wherein the width is one or more widths, and the width is selected from the group consisting of 1× the peak width, 3× the peak width, 5× the peak width, and combinations thereof.

Embodiment 84. The non-transitory computer-readable medium of Embodiment 67, wherein the probability mass percentage is at least about 95%.

Embodiment 85. A system for ascertaining differential accessibility of transcription factor binding motifs in open chromatin regions of cells using a cell barcode genomic sequence dataset, the system comprising: a data store configured to store the cell barcode genomic sequence dataset comprising a plurality of fragment sequence reads and associated barcodes; a computing device communicatively connected to the data store, comprising a clustering engine configured to: receive the cell barcode genomic sequence dataset, align each of the plurality of fragment sequence reads to a reference sequence, identify one or more peaks defined by the aligned plurality of fragment sequence reads, each peak representing an enrichment of the aligned fragment sequence reads at a given position on the reference sequence, wherein peaks that produce a signal above a pre-set signal threshold demarcates an open chromatin region, generate a peak-barcode matrix that is comprised of peaks for each cell barcode, and cluster cells with peaks having similar chromatin accessibility profiles based on one or more given parameters into a cell cluster to form one or more cell clusters, a TF Barcode Matrix engine configured to generate a transcription factor (TF) barcode matrix that maps each peak in the peak-barcode matrix to one or more given TF binding motif(s), and a differential analysis engine configured to: perform a differential accessibility analysis to identify differences in accessibility of one or more peaks and TF binding motifs associated with each identified cell cluster relative to all other identified cell clusters, and generate an output of one or more refined cell clusters based on the differential accessibility analysis, wherein the output comprises a differential accessibility of gene regulatory function and/or specific TF binding motifs associated with each refined cell cluster; and a display communicatively connected to the computing device and configured to display a report containing the output of one or more refined cell clusters.

Embodiment 86. The system of Embodiment 85, wherein the data store and the computing device are part of an integrated apparatus.

Embodiment 87. The system of Embodiment 85, wherein the data store is hosted by a different device than the computing device.

Embodiment 88. The system of Embodiment 85, wherein the data store and the computing device are part of a distributed network system.

Embodiment 89. The system of Embodiment 85, further comprising visualizing the dataset and/or one or more outputs.

Embodiment 90. The system of Embodiment 89, the output comprises differential accessibility of gene regulatory function and/or specific TF binding motifs associated with each refined cell cluster.

Embodiment 91. The system of Embodiment 85, wherein the aligned fragment sequence reads are generated by transposase enzyme cuts during one or more transposition events in one or more accessible open chromatin regions mapped to an identified peak along the reference sequence.

Embodiment 92. The system of Embodiment 85, wherein aligning the fragment sequence reads to the reference sequence further comprises trimming adapter and/or primer oligonucleotide sequences from one or both ends of the fragment sequence reads.

Embodiment 93. The system of Embodiment 85, wherein the reference sequence comprises one or more reference genome sequences, wherein the one or more reference genome sequences include associated genome annotation.

Embodiment 94. The system of Embodiment 93, wherein the one or more reference genome sequences include single species or multi-species reference genome sequences.

Embodiment 95. The system of Embodiment 85, wherein the identified peaks within a given base-pair (bp) length of each other are merged.

Embodiment 96. The system of Embodiment 95, wherein the given bp length is within a range of 100 bp to 1000 bp.

Embodiment 97. The system of Embodiment 96, wherein the given bp length is 500 bp.

Embodiment 98. The system of Embodiment 85, wherein the identifying peaks comprises correcting for GC content bias in the identified peaks.

Embodiment 99. The system of Embodiment 85, wherein generating the peak-barcode matrix comprises: (a) generating a raw peak-barcode matrix comprising peaks for each barcode for all barcodes; and (b) generating a filtered peak-barcode matrix by filtering out non-cell barcodes from the raw peak-barcode matrix.

Embodiment 100. The system of Embodiment 85, wherein the clustering parameter is a closeness of a distance metric calculated using cut sites in the peaks of the cells.

Embodiment 101. The system of Embodiment 85, further comprising at least one of the following: correcting sequencing errors in barcodes in the fragment sequence reads; removing duplicate fragment sequence reads, wherein the removed duplicate fragment sequence reads include reads arising as a consequence of sequencing and/or PCR amplification; and selecting fragment sequence reads that maps with a pre-set mapping quality (MAPQ) score, are not mitochondrial sequences, and/or are not chimerically mapped.

Embodiment 102. The system of Embodiment 101, wherein the pre-set MAPQ score comprises a MAPQ score of 30 or more.

Embodiment 103. The system of Embodiment 85, further comprising associating genes and identifying TF binding motif matches for each identified peak.

Embodiment 104. The system of Embodiment 85, further comprising reducing dimensionality on the peak-barcode matrix.

Embodiment 105. The system of Embodiment 104, wherein reducing dimensionality comprises a selection of a dimensionality reduction technique.

Embodiment 106. The system of Embodiment 105, wherein the dimensionality reduction technique is selected from the group consisting of Principal Component Analysis (PCA), Latent Semantic Analysis (LSA), Probabilistic Latent Semantic Analysis (PLSA), and combinations thereof.

Embodiment 107. The system of Embodiment 106, further comprising applying a graph-clustering technique comprising one of K-means clustering, Spherical k-means clustering, and k-medoids clustering.

Embodiment 108. The system of Embodiment 104, further comprising visualizing the dataset via t-SNE projection.

Embodiment 109. The system of Embodiment 85, wherein the differential accessibility analysis uses a Negative Binomial (NB2) generalized linear model (GLM).

Embodiment 110. The system of Embodiment 85, wherein the system is further configured to generate peak calls from the barcode genomic sequence dataset by: determining a cumulative signal at each position along a signal track of the genome across a constant, moving window; applying an initial threshold on the signal track; applying a multi-component mixture model to the dataset, wherein an expectation maximum is initiated on the data set and converged to a final distribution; generating a global threshold as a probability of the components of the multi-component mixture model; and applying the global threshold across a full data set for making peak calls.

Embodiment 111. The system of Embodiment 110, wherein the system is further configured to generate peak calls from the barcode genomic sequence dataset by: applying a masking threshold on a portion of the dataset, wherein the threshold is a percentage value, wherein all counts above the threshold are masked and all counts below the threshold are compiled into a data subset; determining a first maximum likelihood fitting of a first discrete probability distribution for the data subset; identifying a first residual signal data set from the first maximum likelihood fitting; determining a second maximum likelihood fitting of a second discrete probability distribution on the first residual data set; generating a truncated data set from at least one of the first and second likelihood fittings; applying a first expectation maximization on the truncated data set to fit the truncated data set to background noise data; applying a second expectation maximization on the full data set to generate a final distribution; and generating the global threshold as a function of the first and second discrete probability distributions.

Embodiment 112. The system of Embodiment 110, wherein the system is further configured to generate peak calls from the barcode genomic sequence dataset by: performing a wavelet transform on a local signal within the data to produce a wavelet transformed signal; identifying a putative peak as a local maxima of the wavelet transformed signal; bounding the putative peak at a maximal prominence percentage of the putative peak; performing a loop through a putative peak region, including calculating a beta distribution across the putative peak region; determining a local threshold as a function of signal to noise ratio of the putative peak region; calling the putative peak as a true peak when a probability mass percentage of the beta distribution is above the local threshold.

Embodiment 113. The system of Embodiment 110, wherein the constant, moving window has a length of about 400 bp.

Embodiment 114. The system of Embodiment 110, wherein the multi-component mixture model is a 3-component mixture model, wherein the components are discrete probability distributions selected from the group consisting of a zero-inflated noise component, a negative binomial noise component, and a combination thereof.

Embodiment 115. The system of Embodiment 110, wherein the global threshold is a fixed tail probability of the components of the multi-component mixture model.

Embodiment 116. The system of Embodiment 111, wherein the masking threshold is between about 5% to about 50%.

Embodiment 117. The system of Embodiment 111, wherein the first discrete probability distribution is a zero-inflated noise component with a single geometric distribution.

Embodiment 118. The system of Embodiment 111, wherein the identifying of the first residual signal data set comprises: generating weighted counts for the subset based on the determined first maximum likelihood fitting, and extracting data exceeding the fit as the first residual signal data set.

Embodiment 119. The system of Embodiment 111, wherein the second discrete probability distribution is a first negative binomial noise component.

Embodiment 120. The system of Embodiment 111, wherein the first expectation maximization includes applying a 2-component mixture model on the truncated data set.

Embodiment 121. The system of Embodiment 111, wherein the second expectation maximization includes applying a 3-component mixture model on the truncated data set.

Embodiment 122. The system of Embodiment 112, further comprising performing a wavelet transform on the local signal, with a fixed wavelet width, within the data to produce a wavelet transformed signal.

Embodiment 123. The system of Embodiment 122, wherein the fixed wavelet width is between about 50 bp to about 2000 bp.

Embodiment 124. The system of Embodiment 123, wherein the fixed wavelet width is about 300 bp.

Embodiment 125. The system of Embodiment 124, wherein the maximal prominence percentage is between about 50% and about 95%.

Embodiment 126. The system of Embodiment 125, wherein the maximal prominence percentage is 95%.

Embodiment 127. The system of Embodiment 112, wherein performing a loop through the putative peak region comprises: calculating a first median real signal inside the putative peak region as the peak signal; calculating a second median real signal at a given width in the putative peak region but outside all other observed putative peaks; and calculating the beta distribution with a prior and a Bayesian update.

Embodiment 128. The system of Embodiment 127, wherein the width is one or more widths, and the width is selected from the group consisting of 1× the peak width, 3× the peak width, 5× the peak width, and combinations thereof.

Embodiment 129. The system of Embodiment 112, wherein the probability mass percentage is at least about 95%.

Embodiment 130. A method for generating peak calls from a barcode genomic sequence dataset, the generating of peak calls comprising: determining a cumulative signal at each position along a signal track of the genome across a constant, moving window; applying an initial threshold on the signal track; applying a multi-component mixture model to the dataset, wherein an expectation maximum is initiated on the data set and converged to a final distribution; generating a global threshold as a probability of the components of the multi-component mixture model; and applying the global threshold across a full data set for making peak calls.

Embodiment 131. The method of Embodiment 130, further comprising: applying a masking threshold on a portion of the dataset, wherein the threshold is a percentage value, wherein all counts above the threshold are masked and all counts below the threshold are compiled into a data subset; determining a first maximum likelihood fitting of a first discrete probability distribution for the data subset; identifying a first residual signal data set from the first maximum likelihood fitting; determining a second maximum likelihood fitting of a second discrete probability distribution on the first residual data set; generating a truncated data set from at least one of the first and second likelihood fittings; applying a first expectation maximization on the truncated data set to fit the truncated data set to background noise data; applying a second expectation maximization on the full data set to generate a final distribution; and generating the global threshold as a function of the first and second discrete probability distributions.

Embodiment 132. The method of Embodiment 130, further comprising: performing a wavelet transform on a local signal within the data to produce a wavelet transformed signal; identifying a putative peak as a local maxima of the wavelet transformed signal; bounding the putative peak at a maximal prominence percentage of the putative peak; performing a loop through a putative peak region, including calculating a beta distribution across the putative peak region; determining a local threshold as a function of signal to noise ratio of the putative peak region; calling the putative peak as a true peak when a probability mass percentage of the beta distribution is above the local threshold.

Embodiment 133. The method of Embodiment 130, wherein the constant, moving window has a length of about 400 bp.

Embodiment 134. The method of Embodiment 130, wherein the multi-component mixture model is a 3-component mixture model, wherein the components are discrete probability distributions selected from the group consisting of a zero-inflated noise component, a negative binomial noise component, and a combination thereof.

Embodiment 135. The method of Embodiment 130, wherein the global threshold is a fixed tail probability of the components of the multi-component mixture model.

Embodiment 136. The method of Embodiment 131, wherein the masking threshold is between about 5% to about 50%.

Embodiment 137. The method of Embodiment 131, wherein the first discrete probability distribution is a zero-inflated noise component with a single geometric distribution.

Embodiment 138. The method of Embodiment 131, wherein the identifying of the first residual signal data set comprises: generating weighted counts for the subset based on the determined first maximum likelihood fitting, and extracting data exceeding the fit as the first residual signal data set.

Embodiment 139. The method of Embodiment 131, wherein the second discrete probability distribution is a first negative binomial noise component.

Embodiment 140. The method of Embodiment 131, wherein the first expectation maximization includes applying a 2-component mixture model on the truncated data set.

Embodiment 141. The method of Embodiment 131, wherein the second expectation maximization includes applying a 3-component mixture model on the truncated data set.

Embodiment 142. The method of Embodiment 132, further comprising performing a wavelet transform on the local signal, with a fixed wavelet width, within the data to produce a wavelet transformed signal.

Embodiment 143. The method of Embodiment 142, wherein the fixed wavelet width is between about 50 bp to about 2000 bp.

Embodiment 144. The method of Embodiment 143, wherein the fixed wavelet width is about 300 bp.

Embodiment 145. The method of Embodiment 132, wherein the maximal prominence percentage is between about 50% and about 95%.

Embodiment 146. The method of Embodiment 145, wherein the maximal prominence percentage is 95%.

Embodiment 147. The method of Embodiment 132, wherein performing a loop through the putative peak region comprises: calculating a first median real signal inside the putative peak region as the peak signal; calculating a second median real signal at a given width in the putative peak region but outside all other observed putative peaks; and calculating the beta distribution with a prior and a Bayesian update;

Embodiment 148. The method of Embodiment 147, wherein the width is one or more widths, and the width is selected from the group consisting of 1× the peak width, 3× the peak width, 5× the peak width, and combinations thereof.

Embodiment 149. The method of Embodiment 132, wherein the probability mass percentage is at least about 95%.

What is claimed is:

1. A method for ascertaining differential accessibility of transcription factor (TF) binding motifs in open chromatin regions of cells using a cell barcode genomic sequence dataset, the method comprising:
receiving, by one or more processors, the cell barcode genomic sequence dataset, wherein the dataset comprises a plurality of fragment sequence reads and associated barcodes;
aligning, by the one or more processors, each of the plurality of fragment sequence reads to a reference sequence;
identifying, by the one or more processors, one or more peaks defined by the aligned plurality of fragment sequence reads, each peak representing an enrichment of the aligned fragment sequence reads at a given position on the reference sequence, wherein peaks that produce a signal above a pre-set signal threshold demarcates an open chromatin region;
generating, by the one or more processors, a peak-barcode matrix that is comprised of peaks for each cell barcode;
clustering, by the one or more processors, cells with peaks having similar chromatin accessibility profiles based on one or more given parameters into a cell cluster to form one or more cell clusters;
generating, by the one or more processors, a transcription factor (TF) barcode matrix that maps each peak in the peak-barcode matrix to one or more given TF binding motif(s);
performing, by the one or more processors, a differential accessibility analysis, wherein the analysis identifies differences in accessibility of one or more peaks and TF binding motifs associated with each identified cell cluster relative to all other identified cell clusters; and
generating, by the one or more processors, an output of one or more refined cell clusters based on the differential accessibility analysis, wherein the output comprises a differential accessibility of gene regulatory function and/or specific TF binding motifs associated with each refined cell cluster.

2. The method of claim 1, further comprising visualizing the dataset and/or one or more outputs of claim 1.

3. The method of claim 2, the output comprises differential accessibility of gene regulatory function and/or specific TF binding motifs associated with each refined cell cluster.

4. The method of claim 1, wherein the aligned fragment sequence reads are generated by transposase enzyme cuts during one or more transposition events in one or more accessible open chromatin regions mapped to an identified peak along the reference sequence.

5. The method of claim 1, wherein aligning the fragment sequence reads to the reference sequence further comprises trimming adapter and/or primer oligonucleotide sequences from one or both ends of the fragment sequence reads.

6. The method of claim 1, wherein the reference sequence comprises one or more reference genome sequences, wherein the one or more reference genome sequences include associated genome annotation.

7. The method of claim 6, wherein the one or more reference genome sequences include single species or multi-species reference genome sequences.

8. The method of claim 1, wherein the identified peaks within a given base-pair (bp) length of each other are merged.

9. The method of claim 8, wherein the given bp length is within a range of 100 bp to 1000 bp.

10. The method of claim 9, wherein the given bp length is 500 bp.

11. The method of claim 1, wherein the identifying peaks comprises correcting for GC content bias in the identified peaks.

12. The method of claim 1, wherein generating the peak-barcode matrix comprises: (a) generating a raw peak-barcode matrix comprising peaks for each barcode for all barcodes; and (b) generating a filtered peak-barcode matrix by filtering out non-cell barcodes from the raw peak-barcode matrix.

13. The method of claim 1, wherein the clustering parameter is a closeness of a distance metric calculated using the cut sites in the peaks of the cells.

14. The method of claim 1, further comprising at least one of the following:
correcting sequencing errors in barcodes in the fragment sequence reads;
removing duplicate fragment sequence reads, wherein the removed duplicate fragment sequence reads include reads arising as a consequence of sequencing and/or PCR amplification; and
selecting fragment sequence reads that maps with a pre-set mapping quality (MAPQ) score, are not mitochondrial sequences, and/or are not chimerically mapped.

15. The method of claim 14, wherein the pre-set MAPQ score comprises a MAPQ score of 30 or more.

16. The method of claim 1, further comprising associating genes and identifying TF binding motif matches for each identified peak.

17. The method of claim 1, further comprising reducing dimensionality on the peak-barcode matrix.

18. The method of claim 17, wherein reducing dimensionality comprises a selection of a dimensionality reduction technique.

19. The method of claim 18, wherein the dimensionality reduction technique is selected from the group comprising Principal Component Analysis (PCA), Latent Semantic Analysis (LSA), Probabilistic Latent Semantic Analysis (PLSA), and combinations thereof.

20. The method of claim 19, further comprising applying a graph-clustering technique comprising one of K-means clustering, Spherical k-means clustering, and k-medoids clustering.

21. The method of claim 17, further comprising visualizing the dataset via t-SNE projection.

22. The method of claim 1, wherein the differential accessibility analysis uses a Negative Binomial (NB2) generalized linear model (GLM).

23. A non-transitory computer-readable medium in which a program is stored for causing a computer to perform a method for ascertaining differential accessibility of transcription factor (TF) binding motifs in open chromatin regions of cells using a cell barcode genomic sequence dataset, the method comprising:
receiving, by one or more processors, the cell barcode genomic sequence dataset, wherein the dataset comprises a plurality of fragment sequence reads and associated barcodes;
aligning, by the one or more processors, each of the plurality of fragment sequence reads to a reference sequence;
identifying, by the one or more processors, one or more peaks defined by the aligned plurality of fragment sequence reads, each peak representing an enrichment of the aligned fragment sequence reads at a given position on the reference sequence, wherein peaks that produce a signal above a pre-set signal threshold demarcates an open chromatin region;
generating, by the one or more processors, a peak-barcode matrix that is comprised of peaks for each cell barcode;
clustering, by the one or more processors, cells with peaks having similar chromatin accessibility profiles based on one or more given parameters into a cell cluster to form one or more cell clusters;
generating, by the one or more processors, a transcription factor (TF) barcode matrix that maps each peak in the peak-barcode matrix to one or more given TF binding motif(s);
performing, by the one or more processors, a differential accessibility analysis, wherein the analysis identifies differences in accessibility of one or more peaks and TF binding motifs associated with each identified cell cluster relative to all other identified cell clusters; and
generating, by the one or more processors, an output of one or more refined cell clusters based on the differential accessibility analysis, wherein the output comprises a differential accessibility of gene regulatory function and/or specific TF binding motifs associated with each refined cell cluster.

24. A system for ascertaining differential accessibility of transcription factor binding motifs in open chromatin regions of cells using a cell barcode genomic sequence dataset, the system comprising:
a data store configured to store the cell barcode genomic sequence dataset comprising a plurality of fragment sequence reads and associated barcodes;
a computing device communicatively connected to the data store, comprising,
a clustering engine configured to:
receive the cell barcode genomic sequence dataset,
align each of the plurality of fragment sequence reads to a reference sequence,
identify one or more peaks defined by the aligned plurality of fragment sequence reads, each peak representing an enrichment of the aligned fragment sequence reads at a given position on the reference sequence, wherein peaks that produce a signal above a pre-set signal threshold demarcates an open chromatin region,
generate a peak-barcode matrix that is comprised of peaks for each cell barcode, and
cluster cells with peaks having similar chromatin accessibility profiles based on one or more given parameters into a cell cluster to form one or more cell clusters,
a TF Barcode Matrix engine configured to generate a transcription factor (TF) barcode matrix that maps each peak in the peak-barcode matrix to one or more given TF binding motif(s), and
a differential analysis engine configured to:
perform a differential accessibility analysis to identify differences in accessibility of one or more peaks and TF binding motifs associated with each identified cell cluster relative to all other identified cell clusters, and
generate an output of one or more refined cell clusters based on the differential accessibility analysis, wherein the output comprises a differential accessibility of gene regulatory function and/or specific TF binding motifs associated with each refined cell cluster; and
a display communicatively connected to the computing device and configured to display a report containing the output of one or more refined cell clusters.
